(19) United States
(12) Patent Application Publication
(10) Pub. No.: US 2025/0210193 A1
(43) Pub. Date: Jun. 26, 2025

(54) METHODS AND SYSTEMS FOR MICROBIAL TUMOR HYPOXIA DIAGNOSTICS AND THERANOSTICS

(71) Applicant: The Regents of the University of California, Oakland, CA (US)

(72) Inventor: Gregory Poore, La Jolla, CA (US)

(21) Appl. No.: 18/847,636

(22) PCT Filed: Mar. 15, 2023
(86) PCT No.: PCT/US2023/015250

§ 371 Date: Sep. 16, 2024

Related U.S. Application Data
(60) Provisional application No. 63/320,901, filed on Mar. 17, 2022.
(60) Provisional application No. 63/320,606, filed on Mar. 16, 2022.

(51) Int. Cl. G16H 50/20; C12Q 1/6886; G16B 25/10; G16B 40/20

(52) U.S. Cl. CPC G16H 50/20; C12Q 1/6886; G16B 25/10; G16B 40/20

(57) ABSTRACT

Provided are compositions, methods, and systems for microbial tumor hypoxia diagnostics and theranostics. Specifically described herein are methods of leveraging the oxygen preference of microbial communities to determine unique identifying aspects of tumors.

### CROSS-REFERENCE

This application claims the benefit of U.S. Provisional Application No. 63/320,606 filed on Mar. 16, 2022 and U.S. Provisional Application No. 63/320,901 filed on Mar. 17, 2022, the entirety of which are incorporated herein by reference in its entirety.

### STATEMENT AS TO FEDERALLY SPONSORED RESEARCH

This invention was made with government support under Grant F30 CA243480 awarded by the National Cancer Institute. The government has certain rights in the invention.

### TECHNICAL FIELD

The present invention relates to cancer diagnostics and therapies.

### BACKGROUND

Spatial oxygen gradients are a common feature of solid tissues among most, if not all, cancer types (Bhandari et al. 2019. Nature Genetics). Oxygen availability, and the lack thereof, drives spatial organization and metabolic capacities of cancer cells, such as oxidative phosphorylation in normoxic regions and anaerobic lactogenesis in hypoxic regions, and local pH of these regions (Petrova et al. 2018. Oncogenesis). Moreover, cancer cells in hypoxic tumor regions can share metabolites (e.g., lactate) with cancer cells in normoxic tumor regions in order to enhance overall energy harvest—this process has been called “metabolic symbiosis” (Semenza. 2008. The Journal of Clinical Investigation). Additionally, the degree of hypoxia in an individual tumor has been associated with differential patient survival, tumor staging, and multi-omic aberrations (e.g., mutations, carcinogenic transcriptional regulation) in cancer cells (Bhandari et al. 2019. Nature Genetics; Bhandari et al. 2020. Nature Communications). Furthermore, since hypoxia is a common feature in cancer and excessive hypoxia causes cancer cell death, molecular targets or strategies that increase tumor hypoxia, such as angiogenic inhibitors, have been designed and used to treat cancer (e.g., bevacizumab in colorectal cancer and lung cancer). In short, oxygen (and pH) gradients exist in most cancers, shape how tumors function, prognose patient survival, and are important for cancer drug design and function.

Current methodologies of determining tumor and/or intratumoral oxygenation rely on host molecular markers, physical oxygen concentration measurements, and/or specialized radiometric imaging (Raleigh et al. 1996. Semin. Radiat. Oncol.; Colliez et al. 2017. Front. Oncol.). These methods generally require direct access to the tissue and thus are invasive, involve harmful ionizing radiation exposure to the patient, and/or expensive specialized imaging equipment. Therefore there exists an unmet need for a minimally/non-invasive technique to measure tumor and/or intratumoral oxygenation.

### SUMMARY

The disclosure provides methods for determining the degree of tumor hypoxia and its prognostic correlates based on intratumoral or liquid biopsy-derived microbial compositions and functions. The invention provides that microbial abundances and microbial functional information can predict hypoxic vs. normoxic tumors.

Recent research has led the largest analysis to date of bacteria in tumors, profiling bacterial DNA and RNA in the tissue and blood from more than 10,000 patients with cancer across 33 cancer types and 18,116 samples (Poore et al. 2020. Nature). For the first time, this suggested that microbiomes exist in most, if not all, cancer types and that pan-cancer diagnostics can be designed solely using tissue and blood microbiomes (Poore et al. 2020. Nature). However, the primary question of why certain microbes thrive in certain cancer types has remained unknown and of great interest to researchers and clinicians alike.

Intratumor hypoxic gradients exist that permit local coexistence of aerobes and anaerobes on the scale of millimeters from each other. In natural environments outside of tumors, microbes self-segregate along oxygen and pH gradients and share their metabolites with each other to aid overall ecosystem function—a characteristic very similar to what cancer cells do within the tumor microenvironment. Microbes may act as “oxygen sensors” in tumors, wherein the microbial communities themselves could be assayed to measure (by proxy) the oxygen concentration of the tumor. Moreover, due to the biological and clinical correlation of tumor oxygen concentrations to multiomic cancer cell aberrations, patient survival, and drug design and response (especially angiogenic inhibitors), and because microbial compositions may discriminate very hypoxic versus normoxic tumors, it is contemplated to use them as cancer prognostics and to inform drug design and response.

The invention provides data supporting that aerobic bacteria are most associated with normoxic tumors and anaerobic bacteria with hypoxic tumors using multinomial modelling statistics. The invention contemplates that immunotherapy response efficacy, which is associated with intratumoral microbial compositions (Nejman et al. 2020. Science), may be linked to bacterial populations of varying oxygen tolerances in tumors.

While existing art solely relies on host molecular markers, physical oxygen concentration measurements, and/or specialized radiometric imaging to infer the degree of hypoxia within tumors, these methods generally require direct access to the tissue, radiation exposure to the patient, and/or expensive specialized imaging equipment. Conversely, using microbial biomarkers in tumor tissues and/or liquid biopsies to infer the degree of tumor hypoxia mitigates several of these drawbacks and only requires widely-available sequencing instrumentation. Moreover, since microbial populations can affect angiogenesis (Osherov & Ben-Ami. 2016. PLOS Pathogens), which affects tumor oxygenation, these microbes may comprise engineerable theranostics, which can both sense tumor oxygenation and respond to the degree thereof to affect a treatment to the cancer-bearing host.

Aspects of the disclosure provide a method of determining a tumor oxygen characteristic of a subject. In some embodiments, the method comprises the steps of: (a) receiving one or more biological samples of a subject; (b) sequencing a plurality of nucleic acid molecules of the one or more biological samples, thereby generating a plurality of nucleic acid molecule sequencing reads; (c) mapping the plurality of nucleic acid molecule sequencing reads to a database of microbial genomes, thereby generating a plurality of microbial nucleic acid molecule reads; and (d) determining a tumor oxygen characteristic of the subject as an output of a trained predictive model when the plurality of microbial nucleic acid molecule reads are provided as an input to the trained predictive model. In some embodiments, the tumor comprises breast, lung, bone, brain, pancreas, ovarian, colorectal, skin, or any combination thereof tumors. In some embodiments, the plurality of microbial nucleic acid molecules originate from bacterial obligate aerobes, aerobes, facultative aerobes, microaerophiles, aerotolerants, microaerotolerants, facultative anaerobes, anaerobes, obligate anaerobes, or any combination thereof. In some embodiments, the plurality of nucleic acid molecules comprise microbial DNA, microbial RNA, epigenetic markers on microbial DNA, epigenetic markers on microbial RNA, or any combination thereof. In some embodiments, the plurality of nucleic acid molecules comprises human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

In some embodiments, the method further comprises decontaminating the plurality of nucleic acid molecule sequencing reads thereby producing a plurality of decontaminated nucleic acid molecule sequencing reads. In some embodiments, decontaminating is conducted in silico, using experimental contamination controls, using limit of quantification filtering, or any combination thereof.

In some embodiments, the one or more biological samples comprise a tissue biopsy, a liquid biopsy, or any combination thereof. In some embodiments, the tissue biopsy comprises cancerous tissue, non-cancerous tissue, or any combination thereof. In some embodiments, the liquid biopsy comprises whole blood, red blood cells, plasma, white blood cells, saliva, urine, tears, breast milk, or any combination thereof.

In some embodiments, the trained predictive model comprises a machine learning model. In some embodiments, the trained predictive model comprises a regularized machine learning model. In some embodiments, the trained predictive model comprises one or more machine learning models. In some embodiments, the trained predictive model comprises a gradient boosting machine, neural network, support vector machine, k-means, classification trees, random forest, regression, or any combination thereof machine learning model. In some embodiments, the trained predictive model is trained with microbial DNA, microbial RNA, epigenetic marks on microbial DNA, epigenetic marks on microbial RNA, cell-free microbial RNA, cell-free microbial DNA, non-microbial DNA, non-microbial RNA, epigenetic marks on non-microbial DNA, epigenetic marks on non-microbial RNA, non-microbial cell free DNA, non-microbial cell free RNA, or any combination thereof.

Aspects of the disclosure provide a method of generating a tumor oxygen characteristic predictive model. In some embodiments, the method comprises: (a) obtaining one or more biological samples of one or more subjects with cancer, and corresponding tumor oxygen characteristics of the one or more subjects; (b) sequencing a plurality of nucleic acid molecules of the one or more biological samples thereby generating a plurality of nucleic acid molecule sequencing reads; (c) mapping the plurality of nucleic acid molecule sequencing reads to a database of microbial genomes, thereby generating a plurality of microbial nucleic acid molecule reads; and (d) generating a tumor oxygen characteristic predictive model by training a predictive model with the plurality of microbial nucleic acid molecule reads and corresponding tumor oxygen characteristics of the one or more subjects. In some embodiments, the tumor oxygen characteristic is determined by the RNA expression of one or more genes, the presence or absence of epigenetic marks of one or more genes, the staining intensity of one or more proteins, a physical measurement of oxygen concentration, or any combination thereof. In some embodiments, the tumor and/or cancer of the subject comprises breast, lung, bone, brain, pancreas, ovarian, colorectal, skin, or any combination thereof tumors and/or cancers. In some embodiments, the plurality of microbial nucleic acid molecule reads originate from bacterial obligate aerobes, aerobes, facultative aerobes, microaerophiles, aerotolerants, microaerotolerants, facultative anaerobes, anaerobes, obligate anaerobes, or any combination thereof. In some embodiments, the plurality of nucleic acid molecules comprises microbial DNA, microbial RNA, epigenetic markers on microbial DNA, epigenetic markers on microbial RNA, or any combination thereof. In some embodiments, the plurality of nucleic acid molecules comprises human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

In some embodiments, the method comprises decontaminating the plurality of nucleic acid molecule sequencing reads, thereby producing a plurality of decontaminated nucleic acid molecule sequencing reads. In some embodiments, decontaminating is conducted in silico, using experimental contamination controls, using limit of quantification filtering, or any combination thereof.

In some embodiments, the one or more biological samples comprise a tissue biopsy, a liquid biopsy, or any combination thereof. In some embodiments, the tissue biopsy comprises cancerous tissue, non-cancerous tissue, or any combination thereof. In some embodiments, the liquid biopsy comprises whole blood, red blood cells, plasma, white blood cells, saliva, urine, tears, breast milk, or any combination thereof.

In some embodiments, the predictive model comprises a machine learning model. In some embodiments, the predictive model comprises a regularized machine learning model. In some embodiments, the predictive model comprises one or more machine learning models. In some embodiments, the predictive model comprises a gradient boosting machine, neural network, support vector machine, k-means, classification trees, random forest, regression, or any combination thereof machine learning model.

Aspects of the disclosure provide a method of generating a tumor oxygen characteristic predictive model. In some embodiments, the method comprises: (a) obtaining one or more nucleic acid molecule sequences and corresponding tumor oxygenation characteristics of one or more subjects with cancer from a database; (b) mapping the plurality of nucleic acid molecule sequencing reads to a database of microbial genomes, thereby generating a plurality of microbial nucleic acid molecule reads; and (c) generating a tumor oxygen characteristic predictive model by training a predictive model with the plurality of microbial nucleic acid molecule reads and corresponding tumor oxygen characteristics of the one or more subjects. In some embodiments, the tumor oxygenation characteristics are determined by the RNA expression of one or more genes, the presence of epigenetic marks of one or more genes, the staining intensity of one or more proteins, a physical measurement of oxygen concentration, or any combination thereof. In some embodiments, the cancer of the one or more subjects comprise breast, lung, bone, brain, pancreas, ovarian, colorectal, skin, or any combination thereof cancers. In some embodiments, the plurality of microbial nucleic acid molecule reads originate from bacterial obligate aerobes, aerobes, facultative aerobes, microaerophiles, aerotolerants, microaerotolerants, facultative anaerobes, anaerobes, obligate anaerobes, or any combination thereof. In some embodiments, the one or more nucleic acid molecule sequences comprise microbial DNA, microbial RNA, epigenetic markers on microbial DNA, epigenetic markers on microbial RNA, or any combination thereof. In some embodiments, the one or more nucleic acid molecule sequences comprise sequences of human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

In some embodiments, the method comprises decontaminating the plurality of nucleic acid molecule sequencing reads thereby producing a plurality of decontaminated nucleic acid molecule sequencing reads. In some embodiments, decontaminating is conducted in silico, using experimental contamination controls, using limit of quantification filtering, or any combination thereof.

In some embodiments, the one or more nucleic acid molecule sequences originate from a tissue biopsy, a liquid biopsy, or any combination thereof. In some embodiments, the tissue biopsy comprises cancerous tissue, non-cancerous tissue, or any combination thereof. In some embodiments, the liquid biopsy comprises whole blood, red blood cells, plasma, white blood cells, saliva, urine, tears, breast milk, or any combination thereof.

In some embodiments, the predictive model comprises a machine learning model. In some embodiments, the predictive model comprises a regularized machine learning model. In some embodiments, the predictive model comprises one or more machine learning models. In some embodiments, the predictive model comprises a gradient boosting machine, neural network, support vector machine, k-means, classification trees, random forest, regression, or any combination thereof machine learning models. In some embodiments, the predictive model is configured to provide a prediction of tumor and/or cancer hypoxia of the one or more subjects.

Aspects of the disclosure provide a method of administering a bacterial theranostic. In some embodiments, the method comprises: (a) selecting from a database one or more microbes, where the one or more microbes comprise a metabolic activity based on oxygen concentrations; (b) modifying the one or more microbes with one or more reporter genes, thereby producing a modified one or more microbes, where the one or more reporter genes when incorporated into the one or more microbes, cause the one or more microbes to secrete one or more metabolites in response to oxygen concentrations; and (c) administering to a subject a treatment comprising the one or more modified one or more microbes thereby treating the subject's disease. In some embodiments, the one or more microbes, the one or more metabolites, a product of the one or more reporter genes, or any combination thereof, comprise anticancer properties. In some embodiments, the one or more metabolites or a product of the one or more reporter genes are detected by non-invasive imaging, invasive imaging, or any combination thereof imaging to diagnose the subject's disease. In some embodiments, the one or more metabolites or a product of the one or more reporter genes comprise a second set of molecules configured to be detected by blood based, urine detection, or any combination thereof assays. In some embodiments, the subject's disease comprises cancer. In some embodiments, the treatment comprises an oral available probiotic, an injection into the subject's tumor, an intramuscular injection, an intravenous injection, or any combination thereof.

Aspects of the disclosure provide a method of administering one or more microbes to determine a subject's tumor oxygenation characteristic. In some embodiments, the method comprises: (a) selecting from a database one or more microbes, where the one or more microbes comprise a metabolic activity based on oxygen concentrations; (b) modifying the one or more microbes with one or more reporter genes, where the one or more reporter genes, when incorporated into the one or more microbes, cause the one or more microbes to secrete one or more metabolites or one or more proteins in response to oxygen concentrations; and (c) administering to a subject with a tumor the one or more microbes, where the subject's tumor oxygen characteristic is determined by detecting the one or more secreted metabolites or the one or more proteins of the one or more microbes. In some embodiments, the one or more microbes, the one or more metabolites, the one or more proteins, or any combination thereof, comprise anticancer properties. In some embodiments, the one or more metabolites or proteins are detected by non-invasive imaging, invasive imaging, or any combination thereof imaging to diagnose the subject's disease, tumor oxygenation characteristics, or any combination thereof. In some embodiments, the one or more microbes administered to the subject are administered as an oral available probiotic, an injection into the subject's tumor, an intramuscular injection, an intravenous injection, or any combination thereof. In some embodiments, the one or more metabolites or proteins indicate the prognosis of the subject's disease-free survival, overall survival, likelihood of treatment response, or any combination thereof.

Aspects of the disclosure provide a method of providing a treatment to a set of subjects based on tumor oxygenation characteristics. In some embodiments, the method comprises: (a) receiving a first set of subjects' one or more biological samples and corresponding treatment provided to treat each subject of the first set of subjects' diseases; (b) sequencing the first set of subjects' plurality of nucleic acid molecules of the one or more biological samples thereby producing a plurality of nucleic acid molecule sequencing reads; (c) mapping the first set of subjects' plurality of nucleic acid molecule sequencing reads to a database of microbial genomes, thereby generating a plurality of microbial nucleic acid molecule sequencing reads; (d) training a predictive model with the first set of subjects' plurality of microbial nucleic acid molecule sequencing reads and corresponding treatment provided to each subject of the first set of subjects, thereby generated a trained predictive model; and (e) providing a treatment to treat a second set of subjects' diseases based on the output of the trained predictive model when the trained predictive model is provided, as an input, the second set of subjects' plurality of microbial nucleic acid molecule sequencing reads of the second set of subjects' one or more biological samples. In some embodiments, the predictive model is trained with the first set of subjects' plurality of microbial acid sequencing reads and corresponding oxygen concentration values. In some embodiments, the treatment comprises an anti-angiogenic therapies, non-anti-angiogenic therapies, or any combination thereof treatment. In some embodiments, the first or second set of subjects' diseases comprise cancer. In some embodiments, the first or second set of subjects' plurality of microbial nucleic acid molecule sequencing reads originate from bacterial obligate aerobes, aerobes, facultative aerobes, microaerophiles, aerotolerants, microaerotolerants, facultative anaerobes, anaerobes, obligate anaerobes, or any combination thereof. In some embodiments, the first or second set of subjects' plurality of nucleic acid molecules comprise microbial DNA, microbial RNA, epigenetic markers on microbial DNA, epigenetic markers on microbial RNA, or any combination thereof. In some embodiments, the first or second set of subjects' plurality of nucleic acid molecules comprise human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

In some embodiments, the method comprises decontaminating the first or second set of subjects' plurality of microbial nucleic acid molecule sequencing reads thereby producing a plurality of decontaminated microbial nucleic acid molecule sequencing reads. In some embodiments, decontaminating is conducted in silico, using experimental contamination controls, using limit of quantification filtering, or any combination thereof.

In some embodiments, the first or second set of subjects' one or more biological samples comprise a tissue biopsy, a liquid biopsy, or any combination thereof. In some embodiments, the tissue biopsy comprises cancerous tissue, non-cancerous tissue, or any combination thereof. In some embodiments, the liquid biopsy comprises whole blood, red blood cells, plasma, white blood cells, saliva, urine, tears, breast milk, or any combination thereof.

In some embodiments, the trained predictive model comprises a machine learning model. In some embodiments, the trained predictive model comprises a regularized machine learning model. In some embodiments, the trained predictive model comprises one or more machine learning models, an ensemble of machine learning models, or any combination thereof. In some embodiments, the trained predictive model comprises a gradient boosting machine, neural network, support vector machine, k-means, classification trees, random forest, regression, or any combination thereof machine learning model.

Aspects of the disclosure provide a computer system configured to output an estimate of tumor oxygenation of a subject. In some embodiments, the computer system comprises: (a) one or more processors; and (b) a non-transient computer readable storage medium including software, wherein the software comprises executable instructions that, as a result of execution, cause the one or more processors of the computer system to: (i) receive one or more biological samples of a subject; (ii) sequence a plurality of nucleic acid molecules of the one or more biological samples thereby generating a plurality of nucleic acid molecule sequencing reads; (iii) map the plurality of nucleic acid molecule sequencing reads to a database of microbial genomes, thereby generating a plurality of microbial nucleic acid molecule reads; and (iv) determine an estimate of tumor oxygenation of the subject as an output of a trained predictive model when the plurality of microbial nucleic acid molecule reads are provided as an input to the trained predictive model. In some embodiments, the plurality of microbial nucleic acid molecules originate from bacterial obligate aerobes, aerobes, facultative aerobes, microaerophiles, aerotolerants, microaerotolerants, facultative anaerobes, anaerobes, obligate anaerobes, or any combination thereof. In some embodiments, the plurality of nucleic acid molecules comprises microbial DNA, microbial RNA, epigenetic markers on microbial DNA, epigenetic markers on microbial RNA, or any combination thereof. In some embodiments, the plurality of nucleic acid molecules comprises human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof. In some embodiments, the instructions comprise decontaminate the plurality of nucleic acid molecule sequencing reads thereby producing a plurality of decontaminated nucleic acid molecule sequencing reads. In some embodiments, decontamination is conducted in silico, using experimental contamination controls, or any combination thereof. In some embodiments, the one or more biological samples comprise a tissue biopsy, a liquid biopsy, or any combination thereof. In some embodiments, the tissue biopsy comprises cancerous tissue, non-cancerous tissue, or any combination thereof. In some embodiments, the liquid biopsy comprises whole blood, red blood cells, plasma, white blood cells, saliva, urine, tears, breast milk, or any combination thereof. In some embodiments, the trained predictive model comprises one or more machine learning models, an ensemble of machine learning models, or any combination thereof. In some embodiments, the trained predictive model comprises a regularized machine learning model. In some embodiments, the trained predictive model comprises a gradient boosting machine, neural network, support vector machine, k-means, classification trees, random forest, regression, or any combination thereof machine learning model. In some embodiments, the trained predictive model is trained with microbial DNA, microbial RNA, epigenetic marks on microbial DNA, epigenetic marks on microbial RNA, cell-free microbial RNA, cell-free microbial DNA, non-microbial DNA, non-microbial RNA, epigenetic marks on non-microbial DNA, epigenetic marks on non-microbial RNA, non-microbial cell free DNA, non-microbial cell free RNA, or any combination thereof. In some embodiments, the tumor comprises breast, lung, bone, brain, pancreas, ovarian, colorectal, skin, or any combination thereof cancers.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the ranking distribution of microbes within cancers based on multinomial regression modeling, such that higher ranked microbes (to the right) are more associated with higher tumor hypoxia and lower ranked microbes (to the left) are more associated with lower tumor hypoxia, as described in some embodiments herein.

FIGS. 2A-2D show principal coordinates and permutational multivariate analysis of variance (PERMANOVA) results, revealing the strong influence of varying tumor hypoxia on microbial composition across several cancer types, as described in some embodiments herein.

FIGS. 3A-3C show hypoxia association data that has undergone dimensionality reduction based on weighted or unweighted UniFrac distances with concomitant principal coordinates and PERMANOVA analyses, as described in some embodiments herein.

FIGS. 4A-4B show linear regression correlation data between intratumor microbial richness and Buffa pan-cancer tumor hypoxia scores, as described in some embodiments herein.

FIGS. 5A-5B show linear regression correlation data between phylogenetic alpha diversity of tumors and Buffa pan-cancer tumor hypoxia scores, as described in some embodiments herein.

FIGS. 6A-6B show linear regression correlation data between observed richness, and phylogenetic alpha diversity against Ragnum and Winter pan-cancer hypoxia scores, as described in some embodiments herein.

FIGS. 7A-7B show receiver operating characteristic (ROC) and precision recall (PR) curves of a machine learning model using microbial abundances to discriminate between hypoxic and normoxic tumors, as described in some embodiments herein.

FIGS. 7C-7D show ROC and PR curves of a machine learning model using microbial abundances and permuted labels, thereby providing a negative control analysis, as described in some embodiments herein.

FIG. 8 shows top 10 ranked microbial taxa that discriminate high and low tumor hypoxia, as described in some embodiments herein.

FIGS. 9A-9C show linear correlation data between microbial abundance predicted tumor hypoxia scores and known metagene tumor hypoxia scores, based on machine learning regression, as described in some embodiments herein.

FIGS. 10A-10D show ROC and PR curves for microbial functional data as a predictor of tumor hypoxia using machine learning classifiers based on Kyoto encyclopedia of genes and genomes (KEGG) microbial pathway and module abundances, as described in some embodiments herein.

FIGS. 11A-11D show ROC and PR curves for microbial functional data as a predictor of tumor hypoxia using machine learning classifiers based on microbial ‘metabolic pathways from all domains of life’ (MetaCyc) pathway and module abundances, as described in some embodiments herein.

FIGS. 12A-12C show top ranked functional MetaCyc Pathways (FIG. 12A), KEGG pathways (FIG. 12B), and KEGG modules (FIG. 12C) indicative of high and/or low hypoxia, based on machine learning classifiers using their respective data, as described in some embodiments herein.

FIGS. 13A-13H show intratumoral microbial aerobe, microaerophile, and/or anaerobe relative abundances for patients with breast (FIGS. 13A and 13C), lung (FIGS. 13B and 13D), melanoma (FIG. 13E), pancreas (FIG. 13F) ovary (FIG. 13G), bone (FIG. 13H), glioblastoma (FIG. 13I), and colorectal (FIG. 13J) cancers, as described in some embodiments herein. Note that FIGS. 13A and 13C, as well as FIGS. 13B and 13D, vary only by the stringency of decontamination prior to determination of intratumoral microbial oxygen tolerances and concomitant relative abundances. Note also that the relative abundances comprise all microbes passing decontamination, and those without known oxygen tolerances are categorized as “unknown” in the plots.

FIG. 14 shows a flow diagram of a method of determining a tumor oxygen characteristic of a subject, as described in some embodiments herein.

FIG. 15 shows a flow diagram of a method of generating a tumor hypoxia predictive model from sequencing a biological sample's nucleic acid molecule compositions, as described in some embodiments herein.

FIG. 16 shows a flow diagram of a method of generating a tumor hypoxia predictive model trained on a nucleic acid molecule compositions of biological samples stored on a database, as described in some embodiments herein.

FIG. 17 shows a flow diagram of a method of administering an engineered bacterial theranostic for tumor oxygenation diagnosis and treatment, as described in some embodiments herein.

FIG. 18 shows a flow diagram of a method of administering one or more microbes to determine a subject's tumor oxygenation and/or growth state, as described in some embodiments herein.

FIG. 19 shows a flow diagram of a method of providing a treatment to a set of subjects based on tumor oxygenation characteristics, as described in some embodiments herein.

FIG. 20 shows a flow diagram of a method of providing a treatment to a set of subjects based on a plurality of microbial nucleic acid molecule sequencing reads and a trained predictive model, as described in some embodiments herein.

FIG. 21 shows a system diagram of computer processor configured to implement the methods of the disclosure, as described in some embodiments herein.

FIG. 22 shows a table of genus and species level microbial oxygen tolerances and their prevalence in various forms of cancer, as described in some embodiments herein.

FIG. 23 shows a workflow for determining representative histological sections of cancerous human tissues with a gradient of hypoxia and normoxia, as described in some embodiments herein.

FIG. 24 shows a histologic section stain processing workflow to determine regions of interest (ROIs) or areas of interest (AOIs) to interrogate for spatial RNA based on protein and/or RNA staining that target a presence of human and/or microbial markers, as described in some embodiments herein.

FIG. 25 shows a workflow for spatial RNA and protein analysis of one or more histological sections of cancerous and control human tissue, as described in some embodiments herein.

FIG. 26 shows a workflow for sequencing and processing human and microbial spatial RNA sequence data, as described in some embodiments herein.

FIGS. 27A-27F show quality control data of the sequencing reads for human and microbial spatial RNA sequence data, as described in some embodiments here.

FIGS. 28A-28B show human data of per gene quality control metrics of gene detection rate across AOIs (“Segments”) and histology section type (FIG. 28A), as well as the percent of total genes detected across all segments greater than the limit of quantification (LOQ) (FIG. 28B), as described in some embodiments herein.

FIGS. 29A-29C show UMAP (Uniform Manifold Approximation and Projection for Dimension Reduction) clustering of the human spatial transcriptomics data by histologic section and/or slide (FIG. 29A) and by tissue type (FIG. 29B), and a human gene heat map (FIG. 29C) comprising genes with high coefficients of variation with both slide and tissue type shown, as described in some embodiments herein.

FIGS. 30A-30C show plots comparing the hypoxic scores for each area of interest (AOI) measured for Winter vs. Buffa (FIG. 30A), Ragnum vs. Winter (FIG. 30B), and Buffa vs. Ragnum (FIG. 30C) hypoxic scores, as described in some embodiments herein. Buffa, Winter, and Ragnum each denote distinct gene sets associated with hypoxia, as described in some embodiments herein. Spearman correlations, their magnitude and associated p-values are inset on the plots, as described in some embodiments herein.

FIGS. 31A-31C show plots of single-sample gene set enrichment analysis (ssGSEA) hypoxia scores against Buffa (FIG. 31A), Winter (FIG. 31B), and Ragnum (FIG. 31C) hypoxia scores, and associated chi-squared tests between the third and combined first and second tertile of Buffa, Winter, and Ragnum and the AOIs significantly or not significantly enriched in hypoxia provided by ssGSEA, as described in some embodiments herein. Spearman correlations, their magnitude and associated p-values are inset on the plots, as described in some embodiments herein.

FIGS. 32A-32C show plots of Buffa (FIG. 32A), Winter (FIG. 32B), and Ragnum (FIG. 32C) hypoxia tertiles vs. ssGSEA hypoxia scores, as described in some embodiments herein. Wilcoxon tests are applied on the ssGSEA hypoxia scores across AOIs with respect to the first tertile of Buffa, Winter, and Ragnum hypoxic tertiles, as described in some embodiments herein.

FIGS. 33A-33D shows plots of principal variance component analysis (PVCA) for factors of slide, cancer type, hypoxia, and residual (i.e., other factors that are not associated), for Buffa (FIG. 33A), Winter (FIG. 33B), Ragnum (FIG. 33C) hypoxia tertiles, and ssGSEA binary significant enrichment of hypoxia classification (FIG. 33D), using normalized human gene expression data, as described in some embodiments herein.

FIGS. 34A-34D show plots of fast pre-ranked gene set enrichment analysis (FGSEA) enrichment scores vs. ranked genes of breast cancer AOIs for differentially expressed genes between the third and first tertile of hypoxic scores of Buffa (FIG. 34A), Winter (FIG. 34B), and Ragnum (FIG. 34C), and ssGSEA significant hypoxic enrichment score (FIG. 34D), as described in some embodiments herein.

FIGS. 35A-35D show plots of FGSEA enrichment scores vs. ranked genes of lung cancer AOIs for differentially expressed genes between the third and first tertile of hypoxic scores of Buffa (FIG. 35A), Winter (FIG. 35B), and Ragnum (FIG. 35C), and ssGSEA significant hypoxic enrichment score (FIG. 35D), as described in some embodiments herein.

FIGS. 36A-36D show plots of FGSEA enrichment scores vs. ranked genes of melanoma AOIs for differentially expressed genes between the third and first tertile of hypoxic scores of Buffa (FIG. 36A), Winter (FIG. 36B), and Ragnum (FIG. 36C), and ssGSEA significant hypoxic enrichment score (FIG. 36D), as described in some embodiments herein.

FIGS. 37A-37B show a table of target microbes and associated microbial target type, oxygen tolerance, and microbe name used in spatial RNA transcriptomics analysis, as described in some embodiments herein.

FIG. 38A-38D show microbial sequencing QC (FIG. 38A), probe QC (FIG. 38B), the number of AOIs (i.e., segments) vs. target detection rate of microbes for each tissue type (FIG. 38C), and percentage of AOIs vs. percent of target microbes above limit of quantification (LOQ) filtering (FIG. 38D), as described in some embodiments herein.

FIG. 39 shows a flow diagram of a method of determining a background threshold hyperparameter of limit of quantification (LOQ) filtering by the presence of one or more microbial targets, including a eubacterial target, as described in some embodiments herein.

FIGS. 40A-40C show the unique microbial targets per AOI passing LOQ for each tissue type (FIG. 40A), across slide and tissue type (FIG. 40B), and AOI area normalized across slide and tissue type (FIG. 40C), as described in some embodiments herein.

FIGS. 41A-41F show microbial alpha diversity (i.e., richness) across slide and tissue type (FIG. 41A), tissue type alone (FIG. 41B), and the alpha diversity compared with the Buffa (FIG. 41C), Winter (FIG. 41D), Ragnum (FIG. 41E), and ssGSEA hypoxia scores (FIG. 41F), as described in some embodiments herein.

FIGS. 42A-42F show Shannon entropy of microbial targets across slide and tissue type (FIG. 42A), tissue type alone (FIG. 42B), and the alpha diversity compared with the Buffa (FIG. 42C), Winter (FIG. 42D), Ragnum (FIG. 42E), and ssGSEA hypoxia scores (FIG. 42F), as described in some embodiments herein.

FIGS. 43A-43F show Simpson index of microbial targets across slide and tissue type (FIG. 43A), tissue type alone (FIG. 43B), and the alpha diversity compared with the Buffa (FIG. 43C), Winter (FIG. 43D), Ragnum (FIG. 43E), and ssGSEA hypoxia scores (FIG. 43F), as described in some embodiments herein.

FIG. 44 shows intra-slide alpha diversity (richness) of microbial targets for all slides across all cancerous tissues analyzed compared against Buffa hypoxia scores, as described in some embodiments herein.

FIG. 45 shows intra-slide alpha diversity (richness) of microbial targets for all slides across all cancerous tissues analyzed compared against Winter hypoxia scores, as described in some embodiments herein.

FIG. 46 shows intra-slide alpha diversity (richness) of microbial targets for all slides across all cancerous tissues analyzed compared against Ragnum hypoxia scores, as described in some embodiments herein.

FIG. 47 shows intra-slide alpha diversity (richness) of microbial targets for all slides across all cancerous tissues analyzed compared against ssGSEA hypoxia scores, as described in some embodiments herein.

FIGS. 48A-48B show a plot of AOIs with ssGSEA significantly enriched hypoxia for each slide (FIG. 48A), and corresponding area under the receiver operating characteristic curve (AUROC) (FIG. 48B) of a microbial alpha diversity classifier determining whether a given AOI of a slide is significantly enriched in hypoxia as determined by ssGSEA, as described in some embodiments herein.

FIG. 49 shows a flow diagram of determining receiver operating characteristic (ROC) curves for an alpha diversity classifier determining ssGSEA significantly enriched hypoxia characterization for a given slide, as described in some embodiments herein.

FIGS. 50A-50D show principal coordinate (PCoA) plots for Jaccard (FIG. 50A) and Bray-Curtis (FIG. 50B) distances across different slides, and Jaccard (FIG. 50C) and Bray-Curtis (FIG. 50D) PCoA plots across different tissue types, as described in some embodiments herein. Inset on the plots are permutational multivariate analysis of variance (PERMANOVA) calculations, with pseudo-F values and concomitant p-values, as described in some embodiments herein.

FIGS. 51A-51D show PCoA plots for Jaccard distance calculations of Buffa (FIG. 51A), Winter (FIG. 51B), Ragnum (FIG. 51C), and ssGSEA (FIG. 51D) hypoxia scores across all samples, as described in some embodiments herein. Inset on the plots are PERMANOVA calculations, with pseudo-F values and concomitant p-values, as described in some embodiments herein.

FIGS. 52A-52D show PCoA plots for Bray-Curtis distance calculations of Buffa (FIG. 52A), Winter (FIG. 52B), Ragnum (FIG. 52C), and ssGSEA (FIG. 52D) hypoxia scores across all samples, as described in some embodiments herein. Inset on the plots are PERMANOVA calculations, with pseudo-F values and concomitant p-values, as described in some embodiments herein.

FIGS. 53A-53D show the result of multivariate PERMANOVA analysis of hypoxia scores on AOI microbial composition for Jaccard distances vs. Buffa hypoxia scores, tissue type, and slide (FIG. 53A); Jaccard distances vs. Buffa hypoxia scores and slide with interaction terms calculated (FIG. 53B); Bray-Curtis distances vs. Buffa hypoxia score, tissue type, and slide (FIG. 53C); and Bray-Curtis distances vs. Buffa hypoxia score and slide with interaction terms calculated (FIG. 53D), as described in some embodiments herein.

FIGS. 54A-54D show the result of multivariate PERMANOVA analysis of hypoxia scores on AOI microbial composition for Jaccard distances vs. ssGSEA labels (binary: significantly hypoxic or not), tissue type, and slide (FIG. 54A); Jaccard distances vs. ssGSEA labels and slide with interaction terms calculated (FIG. 54B); Bray-Curtis distances vs. ssGSEA labels, tissue type, and slide (FIG. 54C); and Bray-Curtis distances vs. ssGSEA labels and slide with interaction terms calculated (FIG. 54D), as described in some embodiments herein.

FIG. 55 shows a flow diagram of training a predictive model with normalized, LOQ-filtered microbial compositions to globally predict hypoxic categories across AOIs using a leave-one-out-cross-validation (LOOCV) approach, as described in some embodiments herein.

FIGS. 56A-56D show a gradient boosting machine model classifier trained with LOQ-passing, normalized microbial data in predicting hypoxia of each sample from the upper and lower tertiles of Buffa (FIG. 56A), Winter (FIG. 56B), Ragnum (FIG. 56C) hypoxia scores, and the binary classifications of ssGSEA hypoxia enrichment (FIG. 56D), as described in some embodiments herein. Equivalent models built using scrambled hypoxic labels or shuffled microbial counts of each hypoxic score type are also shown, as described in some embodiments herein.

FIGS. 57A-57D show an elastic net model classifier trained with LOQ-passing, normalized microbial data in predicting hypoxia of each sample from the upper and lower tertiles of Buffa (FIG. 57A), Winter (FIG. 57B), Ragnum (FIG. 57C) hypoxia scores, and the binary classifications of ssGSEA hypoxia enrichment (FIG. 57D), as described in some embodiments herein. Equivalent models built using scrambled hypoxic labels or shuffled microbial counts of each hypoxic score type are also shown, as described in some embodiments herein.

FIGS. 58A-58D show regression analysis plots and associated controls between observed hypoxia scores of Buffa (FIG. 58A), Winter (FIG. 58B), Ragnum (FIG. 58C), and ssGSEA hypoxia enrichment (FIG. 58D), and predicted hypoxia scores, as described in some embodiments herein. Equivalent models built using scrambled hypoxic labels or shuffled microbial counts of each hypoxic score type are also shown, as described in some embodiments herein. Spearman correlations between observed and predicted hypoxia scores, as well as the amount of variation accounted for (R²) and mean absolute error (MAE), are inset on the plots, as described in some embodiments herein. ES, enrichment score.

FIG. 59 shows a flow diagram of decontaminating LOQ-filtered, negative probe normalized microbial data to remove contaminants, as described in some embodiments herein.

FIG. 60 shows a plot of inferred decontamination score (higher means less likely a contaminant) vs. the number of identified taxa, their associated prevalence in AOIs, and the cumulative percent (ECDF) of taxa along the range of decontamination scores, as described in some embodiments herein.

FIG. 61 shows a flow diagram for a leave-one-slide-out (LOSO) machine learning training and validation approach, for classification and/or regression, as described in some embodiments herein.

FIGS. 62A-62E show the number of AOIs on a slide that are classified within a lower third, middle, or upper third tertile of Buffa hypoxia scores and associated AUROC and AUPR for the LOSO trained predictive model discriminating between upper and lower tertiles (FIG. 62A); its AUROC (FIG. 62B) and AUPR (FIG. 62C) performance versus equivalent models using scrambled metadata labels during LOSO training; and its AUROC (FIG. 62D) and AUPR (FIG. 62E) performance versus equivalent models using shuffled microbial count data during LOSO training, as described in some embodiments herein.

FIGS. 63A-63E show the number of AOIs on a slide that are classified within a lower third, middle, or upper third tertile of Winter hypoxia scores and associated AUROC and AUPR for the LOSO trained predictive model discriminating between upper and lower tertiles (FIG. 63A); its AUROC (FIG. 63B) and AUPR (FIG. 63C) performance versus equivalent models using scrambled metadata labels during LOSO training; and its AUROC (FIG. 63D) and AUPR (FIG. 63E) performance versus equivalent models using shuffled microbial count data during LOSO training, as described in some embodiments herein.

FIGS. 64A-64E show the number of AOIs on a slide that are classified within a lower third, middle, or upper third tertile of Ragnum hypoxia scores and associated AUROC and AUPR for the LOSO trained predictive model discriminating between upper and lower tertiles (FIG. 64A); its AUROC (FIG. 64B) and AUPR (FIG. 64C) performance versus equivalent models using scrambled metadata labels during LOSO training; and its AUROC (FIG. 64D) and AUPR (FIG. 64E) performance versus equivalent models using shuffled microbial count data during LOSO training, as described in some embodiments herein.

FIGS. 65A-65E show the number of AOIs on a slide that are classified as significantly enriched in hypoxia by ssGSEA and associated AUROC and AUPR for the LOSO trained predictive model discriminating between upper and lower tertiles (FIG. 65A); its AUROC (FIG. 65B) and AUPR (FIG. 65C) performance versus equivalent models using scrambled metadata labels during LOSO training; and its AUROC (FIG. 65D) and AUPR (FIG. 65E) performance versus equivalent models using shuffled microbial count data during LOSO training, as described in some embodiments herein.

FIG. 66 shows a LOSO gradient boosting machine regression to predict per-AOI hypoxic degree with microbial compositions for each slide and across all tissue types using Buffa hypoxia scores; additionally, the mean average error (MAE) was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

FIG. 67 shows a LOSO gradient boosting machine regression to predict per-AOI hypoxic degree with microbial compositions for each slide and across all tissue types using Winter hypoxia scores; additionally, the MAE was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

FIG. 68 shows a LOSO gradient boosting machine regression to predict per-AOI hypoxic degree with microbial compositions for each slide and across all tissue types using Ragnum hypoxia scores; additionally, the MAE was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

FIG. 69 shows a LOSO gradient boosting machine regression to predict per-AOI hypoxic degree with microbial compositions for each slide and across all tissue types using ssGSEA hypoxia scores; additionally, the MAE was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

FIG. 70 shows a LOSO Bayesian ridge regression (i.e., bridge regression) to predict per-AOI hypoxic degree with microbial compositions for each slide and across all tissue types using Buffa hypoxia scores; additionally, the MAE was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

FIG. 71 shows a LOSO Bayesian ridge regression (i.e., bridge regression) to predict per-AOI hypoxic degree with microbial compositions for each slide and across all tissue types using Winter hypoxia scores; additionally, the MAE was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

FIG. 72 shows a LOSO Bayesian ridge regression (i.e., bridge regression) to predict per-AOI hypoxic degree with microbial compositions for each slide and across all tissue types using Ragnum hypoxia scores; additionally, the MAE was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

FIG. 73 shows a LOSO Bayesian ridge regression (i.e., bridge regression) to predict per-AOI hypoxic degree with microbial compositions for each slide and across all tissue types using ssGSEA hypoxia scores; additionally, the MAE was determined for the LOSO classifier and compared to equivalent LOSO models when hypoxia degree labels were scrambled during LOSO training or the count data was shuffled during LOSO training, as described in some embodiments herein. Spearman correlations and associated p-values per slide are inset on the scatter plots, as described in some embodiments herein. A paired t-test was used to compare the MAE of the actual LOSO models vs. equivalent LOSO models using scrambled labels or shuffled count data, as described in some embodiments herein.

### DETAILED DESCRIPTION

The present disclosure relates, in part, to a pan-cancer microbiome analysis performed on a patient cohort that also contains human molecular information (e.g., DNA variants, RNA abundances, methylation marks, protein abundances). This human molecular information, specifically RNA abundances, can be used to quantify the degree of hypoxia (i.e., lack of oxygen) using known statistical methods (Bhandari et al. 2019. Nature Genetics) in the same tumors processed for microbial compositions and functions. The individual bacteria within the cancer microbiome dataset can be cross-referenced against a database of bacterial phenotypes (BacDive; Söhngen et al. 2016. Nucleic Acids Research) to indicate the oxygen tolerances of bacteria, such as aerobes, microaerophiles, and anaerobes, within each tumor sample. This invention provides methods describing how (i) microbial diversity varies as a function of tumor hypoxia, (ii) hypoxic versus normoxic tumors can be discriminated solely using microbial taxonomic abundances, (iii) hypoxic versus normoxic tumors can be discriminated solely using (microbial) functional pathway abundances, (iv) aerobes, microaerophiles, and anaerobes co-exist in individual tumors, or any combination thereof methods. The invention provides, in some embodiments, further methods of treatment of subjects diagnosed with cancer with pharmaceutical compositions in effective amounts commensurate with the degree of disease. Typically, a subject's tumor hypoxia is measured by host molecular markers, physical oxygen concentration measurements, and/or specialized invasive radiometric imaging, and this invention provides methods describing how tumor hypoxia may also be measured using microbial compositions and/or functions.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention may employ conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (MJ. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and CC. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

### I. Methods

Aspects of the disclosure provided herein comprise methods configured to output a subject and/or a group of subjects' disease oxygen characteristic. In some cases, the disease may comprise cancer and the oxygen characteristic may be of a tumor. In some instances, the tumor oxygen characteristic may comprise a hypoxic, normoxic, gradient, or any combination thereof tumor oxygen characteristic. In some cases, the disease oxygen characteristic may be determined by the microbial abundance of a subject's biological samples. In some cases, the biological samples may comprise a liquid and/or a tissue biopsy. In some instances, the liquid biopsy may comprise whole blood, red blood cells, plasma, white blood cells, saliva, urine, tears, breast milk, or any combination thereof liquid biopsies. In some cases, the tissue biopsy may comprise cancer tissue, non-cancerous tissue, or any combination thereof. The tissue biopsy may comprise a tissue resection, tissue punch biopsy, needle biopsy, or any combination thereof.

Determining Tumor Oxygen Characteristics with Microbial Abundance

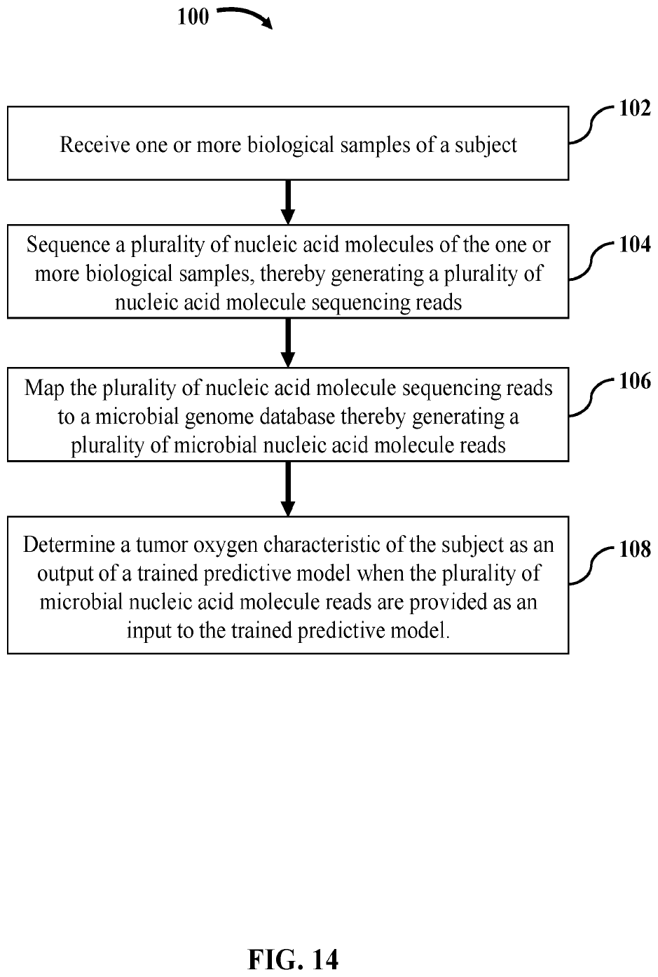

In some embodiments, aspects of the disclosure comprise a method 100, as seen in FIG. 14, of determining a tumor oxygen characteristic of a subject. In some cases the method of determining a tumor oxygen characteristic may comprise the steps of: (a) receiving one or more biological samples of a subject 102; (b) sequencing a plurality of nucleic acid molecules of the one or more biological samples thereby generating a plurality of nucleic acid molecule sequences 104; (c) mapping the plurality of nucleic acid molecule sequencing reads to a microbial genome database thereby generating a plurality of microbial nucleic acid molecule reads 106; and (d) determining a tumor oxygen characteristic of the subject as an output of a trained predictive model when the plurality of microbial nucleic acid molecule reads are provided as an input to the trained predictive model 108. In some cases, the tumor comprises breast, lung, bone, brain, pancreas, ovarian, colorectal, skin cancer, or any combination thereof tumors. In some instances, the plurality of microbial nucleic acid molecules originate from bacterial aerobes, microaerophiles, anaerobes, facultative anaerobes, facultative aerobes, or any combination thereof. In some cases, the plurality of nucleic acid molecules comprise microbial DNA, microbial RNA, epigenetic markers on microbial DNA, epigenetic markers on microbial RNA, human RNA, human DNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, spatially-resolved DNA, spatially-resolved RNA, or any combination thereof nucleic acid molecules.

In some cases, the method 100 may further comprise decontaminating the plurality of nucleic acid molecule sequencing reads thereby producing a plurality of decontaminated nucleic acid molecule sequencing reads. The decontamination of the plurality of nucleic acid molecule sequencing reads may increase the accuracy of the trained predictive models described elsewhere herein. In some cases, decontaminating may be conducted in silico, with experimental contamination controls, using limit of quantification filtering, or any combination thereof methods of decontamination. In some cases in silico decontamination may comprise comparing individual microbial abundances across one or more biological samples of varying analyte concentrations. The one or more contaminate microbes may be identified by a fractional abundance of microbial reads that are inversely proportional to the analyte concentrations of one or more biological samples. For example, at lower analyte concentrations, the contaminate microbes will have a higher fractional read abundance compared to the overall abundance of the microbial nucleic acid molecules. In some instances, such a decontamination method may comprise the steps of: (i) measuring a plurality of analyte concentrations from the one or more biological samples of a subject; (ii) sequencing the plurality of nucleic acid molecules at the plurality of dilutions to generate a plurality of nucleic acid molecule sequences; (iii) mapping the plurality of nucleic acid molecule sequencing reads to a microbial genome database thereby generating a plurality of microbial nucleic acid molecule reads of the plurality of dilutions; (iv) identifying contaminate microbes from the plurality of microbial nucleic acid molecule reads where the contaminate microbes are present with a fractional abundance that is inverse proportional to the plurality of dilutions across one or more biological samples; and (v) removing the contaminate microbes from the microbial nucleic acid molecule reads prior to step (d) 108.

In some instances, decontamination by experimental contamination controls may comprise identifying the presence of microbial nucleic acid molecules within one or more negative control samples (e.g., empty sample collection vessels, vials, dishes, sealable containers, swabs, vials only of reagents, etc.) that may be removed from the plurality of microbial nucleic acid molecules prior to step (d). In some cases, microbes and their microbial nucleic acid molecules are removed if identified in proportionately more negative control samples than biological samples. In some cases, microbes and their microbial nucleic acid molecules are removed on the basis of a statistical test, such as a Fisher exact test, that describes differences in presence proportionality of the microbial nucleic acid molecules between negative controls and biological samples. In some cases, a method of decontamination by experimental contamination controls may comprise the steps of: (i) obtaining one or more negative control vessels or chambers or reagents used to transport and/or store and/or process the one or more biological samples; (ii) sequencing nucleic acid molecules of the one or more negative control vessels, thereby generating a plurality of negative control sequencing reads; (iii) mapping the plurality of negative control sequencing reads to a microbial genome database thereby generating a plurality of microbial nucleic acid molecule reads; and (iv) removing the plurality of negative control microbial nucleic acid molecule reads from the microbial nucleic reads of the one or more biological samples prior to step (d).

In some cases, decontamination may comprise a combination of in silico decontamination and experimental contamination controls.

In some cases, the one or more biological samples may comprise a tissue biopsy, a liquid biopsy, or any combination thereof. In some instances, the tissue biopsy may comprise cancerous tissue, non-cancerous tissue, or any combination thereof. In some cases, the liquid biopsy may comprise whole blood, red blood cells, plasma, white blood cells, saliva, urine, tears, breast milk, or any combination thereof.

In some instances, the trained predictive model may comprise a machine learning model. In some cases, the trained predictive model may comprise a regularized machine learning model. In some instances, the trained predictive model may comprise one or more machine learning models. In some cases, the trained predictive model may comprise a gradient boosting machine, neural network, support vector machine, k-means, classification trees, random forest, regression, or any combination thereof machine learning model. In some instances, the trained predictive model is trained with microbial DNA, microbial RNA, epigenetic marks on microbial DNA, epigenetic marks on microbial RNA, cell-free microbial RNA, cell-free microbial DNA, non-microbial DNA, non-microbial RNA, epigenetic marks on non-microbial DNA, epigenetic marks on non-microbial RNA, non-microbial cell-free DNA, non-microbial cell-free RNA, or any combination thereof.

### Determining Tumor Oxygen Characteristics by Microbial Proteins and Metabolites

Aspects of the disclosure comprise a method of determining a tumor oxygen characteristic of a subject based on microbial analytes. In some cases the method of determining a tumor oxygen characteristic of a subject based on microbial analytes may comprise the steps of: (a) receiving one or more biological samples of a subject; (b) isolating one or more microbial analytes of the one or more biological samples; and (c) determining a tumor oxygen characteristic of the subject as an output of a trained predictive model when the one or more microbial analytes are provided as an input. In some cases, the tumor comprises breast, lung, bone, brain, pancreas, ovarian, colorectal, skin cancer, or any combination thereof tumors. In some instances, one or more microbial analytes may originate from bacterial aerobes, microaerophiles, anaerobes, facultative anaerobes, facultative aerobes, or any combination thereof. In some cases, the one or more microbial analytes may comprise microbial nucleic acid molecules, proteins, metabolites, or any combination thereof.

In some cases the one or more biological samples may comprise a tissue biopsy, a liquid biopsy, or any combination thereof. In some instances, the tissue biopsy may comprise cancerous tissue, non-cancerous tissue, or any combination thereof. In some cases, the liquid biopsy may comprise whole blood, red blood cells, plasma, white blood cells, saliva, urine, tears, breast milk, or any combination thereof.

In some instances, the trained predictive model may comprise a machine learning model. In some cases, the trained predictive model may comprise a regularized machine learning model. In some instances, the trained predictive model may comprise one or more machine learning models. In some cases, the trained predictive model may comprise a gradient boosting machine, neural network, support vector machine, k-means, classification trees, random forest, regression, or any combination thereof machine learning model. In some instances, the trained predictive model is trained with a subject's one or more biological samples' isolated microbial nucleic acid molecules and/or microbial proteins and/or microbial metabolites and corresponding tumor oxygenation characteristic.

In some cases, the methods may be configured to determine a hypoxic state of a subject, or a group of subjects' tumor may involve using and/or training a predictive model that may output a tumor oxygen characteristic score for a subject or a group of subjects. In some instances, the predictive model may comprise a machine learning and/or artificial intelligence model.

### Method of Generating a Tumor Oxygen Characteristic Predictive Model

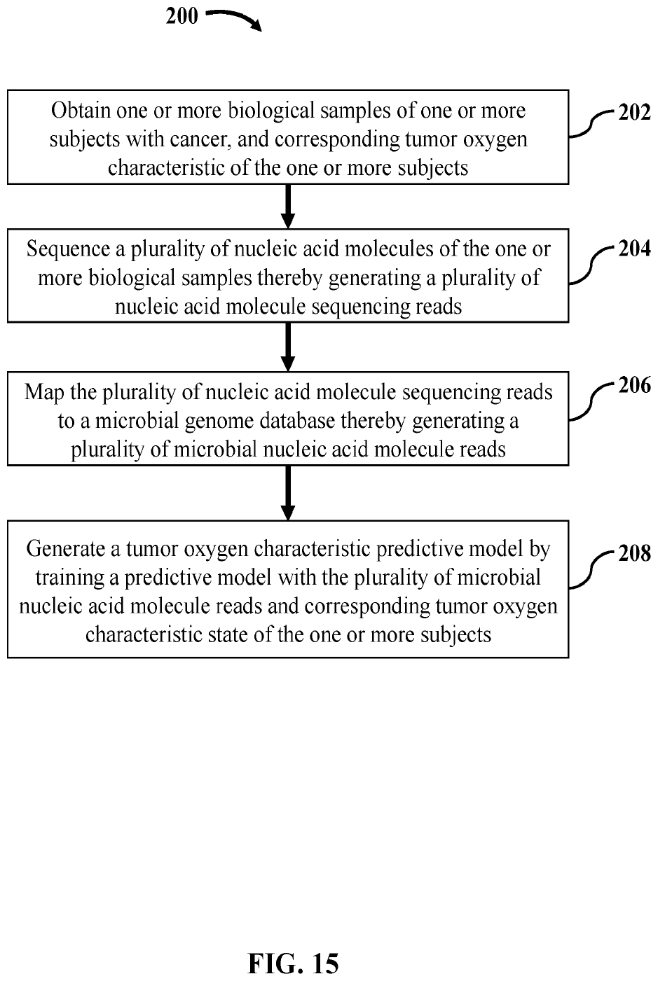

Aspects of the disclosure provided herein comprise a method 200, as seen in FIG. 15, of generating a tumor oxygen characteristic predictive model with subjects' harvested samples. In some instances, the method may comprise the steps of: (a) obtaining one or more biological samples of one or more subjects with cancer, and corresponding tumor oxygen characteristics of the one or more subjects 202; (b) sequencing a plurality of nucleic acid molecules of the one or more biological samples thereby generating a plurality of nucleic acid molecule sequencing reads 204; (c) mapping the plurality of nucleic acid molecule sequencing reads to a microbial database thereby generating a plurality of microbial nucleic acid molecule reads 206; and (d) generating a tumor oxygen characteristic predictive model by training a predictive model with the plurality of microbial nucleic acid molecule reads and corresponding tumor oxygen characteristics of the one or more subjects 208.

In some instances, the cancer of the subject may comprise breast, lung, bone, brain, pancreases, ovarian, colorectal, skin, or any combination thereof cancers. In some cases, the tumor oxygen characteristic is determined by the RNA expression of one or more genes, the abundance of one or more proteins, the presence or absence of epigenetic marks of one or more genes, a protein staining intensity, a physical measurement of oxygen concentration, or any combination thereof. In some cases, the one or more biological samples may comprise a tissue biopsy, a liquid biopsy, or any combination thereof. In some instances, the tissue biopsy may comprise cancerous tissue, non-cancerous tissue, or any combination thereof tissues. In some cases, the liquid biopsy may comprise whole blood, red blood cells, plasma, white blood cells, saliva, urine, tears, breast milk, or any combination thereof liquid biopsies. In some cases, the plurality of microbial nucleic acid molecule reads originate from bacterial aerobes, microaerophiles, anaerobes, facultative anaerobes, facultative aerobes, or any combination thereof. In some cases, the plurality of nucleic acid molecules comprise microbial DNA, microbial RNA, epigenetic markers on microbial DNA, epigenetic markers on microbial RNA, human RNA, human DNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof nucleic acid molecules.

In some instances, the predictive model may comprise a machine learning model. In some cases, the predictive model may comprise a regularized machine learning model. In some instances, the predictive model may comprise one or more machine learning models. In some cases, the predictive model may comprise a gradient boosting machine, neural network, support vector machine, k-means, classification trees, random forest, regression, or any combination thereof machine learning model.

In some cases, the method of generating a tumor oxygen characteristic predictive model 200 may further comprise a step of decontaminating the plurality of nucleic acid molecule sequencing reads thereby producing a plurality of decontaminated nucleic acid molecule sequencing reads. In some instances, decontaminating is conducted in silico, using experimental contamination controls, using limit of quantification filtering, or any combination thereof methods. The decontamination of the plurality of nucleic acid molecule sequencing reads may increase the accuracy of the trained predictive models described elsewhere herein. In some cases, decontaminating may be conducted in silico, with experimental contamination controls, using limit of quantification filtering, or any combination thereof method of decontamination. In some cases in silico decontamination may comprise comparing individual microbial abundances across one or more biological samples of varying analyte concentrations. The one or more contaminate microbes may be identified by a fractional abundance of microbial reads that are inversely proportional to the analyte concentrations of one or more biological samples. For example, at lower analyte concentrations, the contaminate microbes will have a higher fractional read abundance compared to the overall abundance of the microbial nucleic acid molecules. In some instances, such a decontamination method may comprise the steps of: (i) measuring a plurality of analyte concentrations from the one or more biological samples of a subject; (ii) sequencing the plurality of nucleic acid molecules at the plurality of dilutions to generate a plurality of nucleic acid molecule sequences; (iii) mapping the plurality of nucleic acid molecule sequencing reads to a microbial genome database thereby generating a plurality of microbial nucleic acid molecule reads of the plurality of dilutions; (iv) identifying contaminate microbes from the plurality of microbial nucleic acid molecule reads where the contaminate microbes are present with a fractional abundance that is inverse proportional to the plurality of dilutions across one or more biological samples; and (v) removing the contaminate microbes from the microbial nucleic acid molecule reads prior to step (d) 208.

In some instances, decontamination by experimental contamination controls may comprise identifying the presence of microbial nucleic acid molecules within one or more negative control samples (e.g., empty sample collection vessels, vials, dishes, sealable containers, swabs, vials only of reagents, etc.) that may be removed from the plurality of microbial nucleic acid molecules prior to step (d) 208. In some cases, microbes and their microbial nucleic acid molecules are removed if identified in proportionately more negative control samples than biological samples. In some cases, microbes and their microbial nucleic acid molecules are removed on the basis of a statistical test, such as a Fisher exact test, that describes differences in presence proportionality of the microbial nucleic acid molecules between negative controls and biological samples. In some cases, a method of decontamination by experimental contamination controls may comprise the steps of: (i) obtaining one or more negative control vessels or chambers or reagents used to transport and/or store and/or process the one or more biological samples; (ii) sequencing nucleic acid molecules of the one or more negative control vessels, thereby generating a plurality of negative control sequencing reads; (iii) mapping the plurality of negative control sequencing reads to a microbial genome database thereby generating a plurality of microbial nucleic acid molecule reads; and (iv) removing the plurality of negative control microbial nucleic acid molecule reads from the microbial nucleic reads of the one or more biological samples prior to step (d).

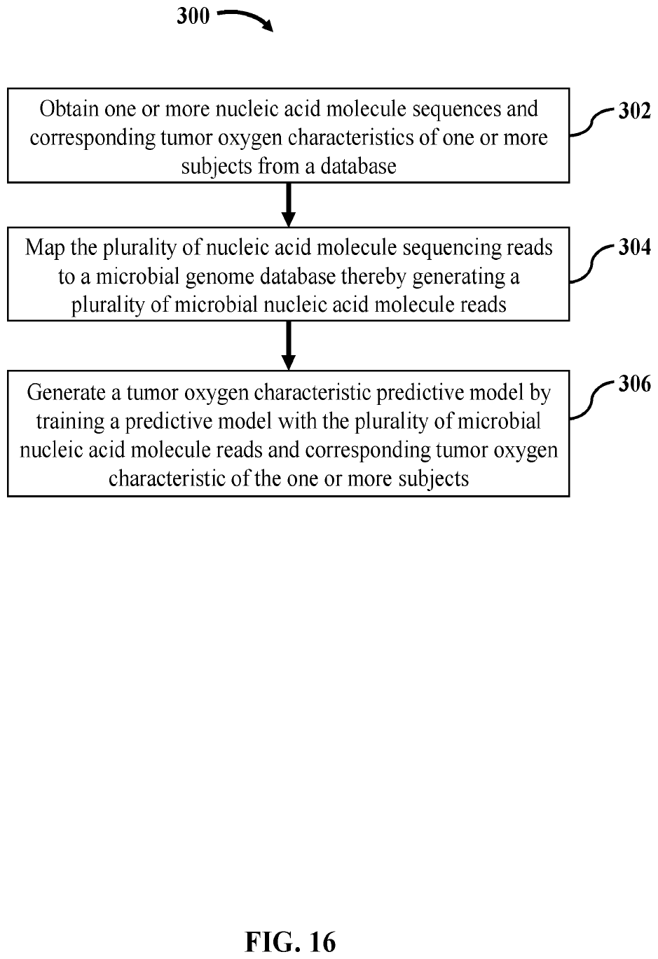

Aspects of the disclosure provided herein comprise a method 300, as seen in FIG. 16, of generating a tumor oxygen characteristic predictive model from a database of subject's one or more sequencing reads. In some cases, the method may comprise the steps in any order: (a) obtaining one or more nucleic acid molecule sequences and corresponding tumor oxygen characteristics of one or more subjects with cancer from a database 302; (b) mapping the plurality of nucleic acid molecule sequencing reads to a microbial genome database thereby generating a plurality of microbial nucleic acid molecule reads 304; and (c) generating a tumor oxygen characteristic predictive model by training a predictive model with the plurality of microbial nucleic acid molecule reads and corresponding tumor oxygen characteristics of the one or more subjects 306. In some cases, the method may further comprise decontaminating the plurality of nucleic acid molecule sequencing reads thereby producing a plurality of decontaminated nucleic acid molecule sequencing reads. In some cases, decontaminating may be conducted in silico, using experimental contamination controls, using limit of quantification filtering, or any combination thereof decontamination approaches.

The decontamination of the plurality of nucleic acid molecule sequencing reads may increase the accuracy of the trained predictive models described elsewhere herein. In some cases, decontaminating may be conducted in silico, with experimental contamination controls, using limit of quantification filtering, or any combination thereof method of decontamination. In some cases in silico decontamination may comprise comparing individual microbial abundances across one or more biological samples of varying analyte concentrations. The one or more contaminate microbes may be identified by a fractional abundance of microbial reads that are inversely proportional to the analyte concentrations of one or more biological samples. For example, at lower analyte concentrations, the contaminate microbes will have a higher fractional read abundance compared to the overall abundance of the microbial nucleic acid molecules. In some instances, such a decontamination method may comprise the steps of: (i) measuring a plurality of analyte concentrations from the one or more biological samples of a subject; (ii) sequencing the plurality of nucleic acid molecules at the plurality of dilutions to generate a plurality of nucleic acid molecule sequences; (iii) mapping the plurality of nucleic acid molecule sequencing reads to a microbial genome database thereby generating a plurality of microbial nucleic acid molecule reads of the plurality of dilutions; (iv) identifying contaminate microbes from the plurality of microbial nucleic acid molecule reads where the contaminate microbes are present with a fractional abundance that is inverse proportional to the plurality of dilutions across one or more biological samples; and (v) removing the contaminate microbes from the microbial nucleic acid molecule reads prior to step (d) 306.

In some instances, decontamination by experimental contamination controls may comprise identifying the presence of microbial nucleic acid molecules within one or more negative control samples (e.g., empty sample collection vessels, vials, dishes, sealable containers, swabs, vials only of reagents, etc.) that may be removed from the plurality of microbial nucleic acid molecules prior to step (d). In some cases, microbes and their microbial nucleic acid molecules are removed if identified in proportionately more negative control samples than biological samples. In some cases, microbes and their microbial nucleic acid molecules are removed on the basis of a statistical test, such as a Fisher exact test, that describes differences in presence proportionality of the microbial nucleic acid molecules between negative controls and biological samples. In some cases, a method of decontamination by experimental contamination controls may comprise the steps of: (i) obtaining one or more negative control vessels or chambers or reagents used to transport and/or store and/or process the one or more biological samples; (ii) sequencing nucleic acid molecules of the one or more negative control vessels, thereby generating a plurality of negative control sequencing reads; (iii) mapping the plurality of negative control sequencing reads to a microbial genome database thereby generating a plurality of microbial nucleic acid molecule reads; and (iv) removing the plurality of negative control microbial nucleic acid molecule reads from the microbial nucleic reads of the one or more biological samples prior to step (d).

In some instances, the cancer of the one or more subjects may comprise one or more tumors. In some cases, the cancer of the one or more subjects may comprise cancer of the breast, lung, bone, brain, pancreas, ovarian, colorectal, skin, or any combination thereof cancers. In some cases, the predictive model may be configured to provide a prediction of a tumor oxygenation characteristic of one or more subjects or groups of subjects.

In some cases, the tumor oxygen characteristic may be determined by RNA expression of one or more genes, the abundance of one or more proteins, the presence of epigenetic marks of one or more genes, the staining of one or more proteins, a physical measurement of oxygen concentration, or any combination thereof. In some cases, the plurality of microbial nucleic acid molecule reads may originate from bacterial aerobes, microaerophiles, anaerobes, facultative anaerobes, facultative aerobes, or any combination thereof. In some cases, the one or more nucleic acid molecule sequences may comprise sequences of microbial DNA, microbial RNA, epigenetic markers on microbial DNA, epigenetic markers on microbial RNA, human RNA, human DNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

In some instances, the predictive model may comprise a machine learning model. In some cases, the predictive model may comprise a regularized machine learning model. In some instances, the predictive model may comprise one or more machine learning models. In some cases, the predictive model may comprise a gradient boosting machine, neural network, support vector machine, k-means, classification trees, random forest, regression, or any combination thereof machine learning model.

In some cases, the one or more nucleic acid molecule sequences may originate from a tissue biopsy, a liquid biopsy, or any combination thereof. In some instances, the tissue biopsy comprises cancerous tissue, non-cancerous tissue, or any combination thereof. In some cases, the liquid biopsy may comprise whole blood, red blood cells, plasma, white blood cells, saliva, urine, tears, breast milk, or any combination thereof.

Aspects of the disclosure provided herein comprise a method of training a tumor oxygen characteristic predictive model from tumor tissue spatial transcriptomics data. In some cases, the method may comprise the following steps in any order: (a) receiving or obtaining a histologic section of a tumor tissue of a subject; (b) performing nucleic acid molecule molecule spatial transcriptomics on the histologic section of the tumor tissue to identify one or more human and/or microbial targets; and (c) generating a tumor oxygen characteristic predictive model with the one or more human and/or microbial targets. In some cases, the method may further comprise filtering the one or more human and/or microbial targets with a background signal threshold determined by one or more negative control spatial transcriptomics probes. In some cases, the negative control spatial transcriptomics probe(s) may comprise a scrambled eubacterial probe. In some cases the background signal threshold may be determined through an iterative process of increasing the background signal threshold until no signal and/or expression of the negative control spatial transcriptomic probes are detected in negative control tissues and/or tissue regions. In some instances, filtering may comprise limit of quantification filtering.

### Administering a Bacterial Theranostic

In some instances, the methods described herein comprise methods to determine a hypoxic state of a subject or a group of subjects' tumor(s) and determine an optimal treatment for the tumor(s) of the subject or group of subjects. In some cases, the methods described herein may comprise a method of administering genetically modified bacteria, where the genetically modified bacteria may secrete one or more detection and/or cancer treatment metabolites when in environments of varying oxygen concentration. In some instances, the oxygen concentration environments may comprise a normoxic, hypoxic, gradient, or any combination thereof oxygen concentration environments.

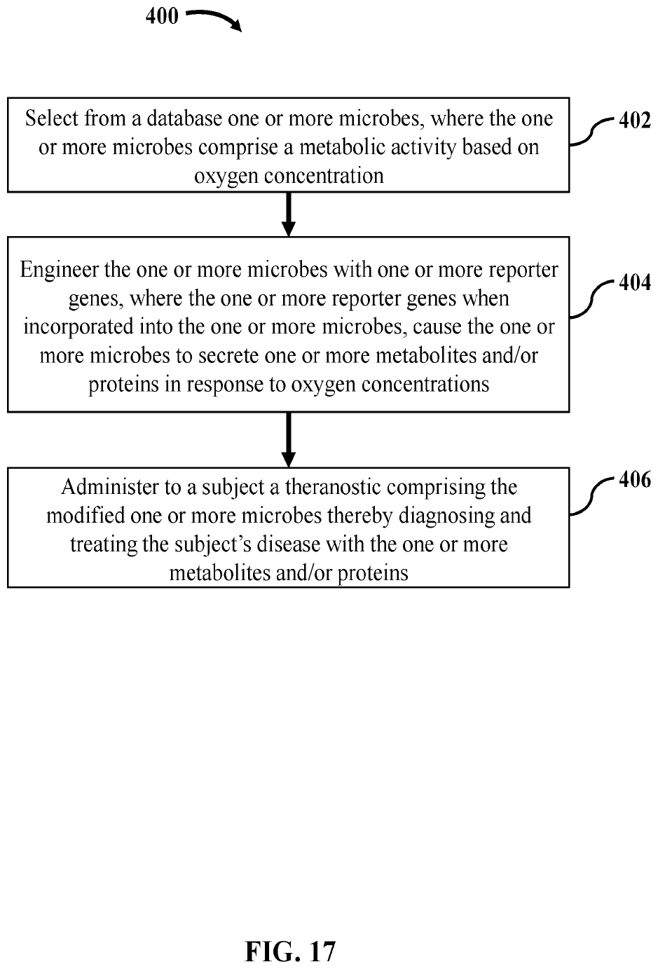

Aspects of the disclosure provided herein may comprise a method 400, as seen in FIG. 17, of administering a microbial theranostic. In some cases, a theranostic may comprise a combination of a therapeutic and diagnostic composition and/or modality. In some instances, the method of administering a microbial theranostic may comprise the steps in any order of: (a) selecting from a database one or more microbes, where the one or more microbes comprise a metabolic activity based on oxygen concentrations 402; (b) engineering the one or more microbes with one or more reporter genes, where the one or more reporter genes when incorporated into the one or more microbes, cause the one or more microbes to secrete one or more metabolites and/or proteins in response to oxygen concentrations 404; and (c) administering to a subject a theranostic comprising the modified one or more microbes thereby diagnosing and treating the subject's disease 406.

In some cases, the one or more theranostic engineered microbes may comprise a self-destruct feature, such as lysis, after therapeutically treating the cancer and/or not having detected a cancer in the subject.

In some cases, the subject's disease may comprise cancer. In some instances, the cancer may comprise cancer of the breast, lung, bone, brain, pancreas, ovarian, colorectal, skin, or any combination thereof cancers. In some instances, the one or more microbes or their metabolites and/or proteins may comprise a first set of metabolites and/or proteins with anticancer properties. In some cases, the one or more metabolites and/or proteins may be detected by non-invasive imaging, invasive imaging, or any combination thereof imaging. In some cases, the one or more metabolites and/or proteins may comprise a second set of metabolites and/or proteins configured to be detected by blood based, urine detection, or any combination thereof assays. In some cases, the treatment may comprise an oral available probiotic, an injection into the subject's cancerous tumor, an intravenous injection, an intramuscular injection, or any combination thereof.

### Administering Genetically Engineered Bacteria for Tumor Oxygen Characteristic Detection

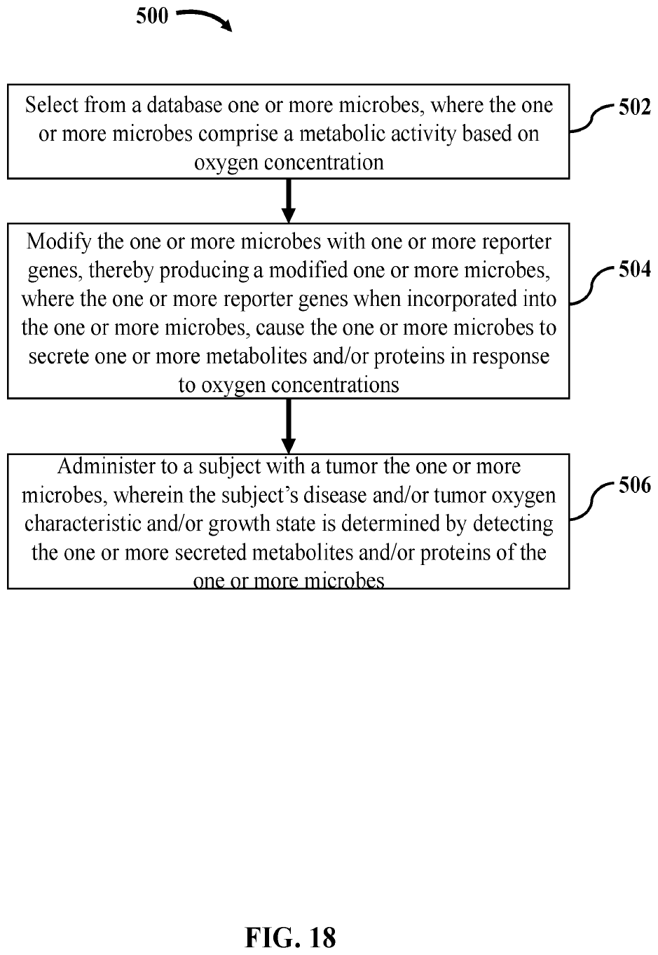

Aspects of the disclosure provided herein comprise a method of administering one or more microbes to determine a subject's tumor oxygen characteristics and/or growth state 500, as seen in FIG. 18. In some cases, the method may comprise the following steps in any order: (a) selecting from a database one or more microbes, where the one or more microbes comprise a metabolic activity based on oxygen concentration 502; (b) modifying the one or more microbes with one or more reporter genes, thereby producing a modified one or more microbes, where the one or more reporter genes when incorporated into the one or more microbes, cause the one or more microbes to secrete one or more metabolites and/or proteins in response to oxygen concentrations 504; and (c) administering to a subject with a tumor the one or more microbes, where the subject's disease and/or tumor oxygen characteristic and/or growth state is determined by detecting the one or more secreted metabolites and/or proteins of the one or more microbes 506.

In some instances, the subject's tumor may comprise cancer of the breast, lung, bone, brain, pancreas, ovarian, colorectal, skin, or any combination thereof cancers. In some instances, the one or more microbes or their one or more metabolites may comprise anticancer properties. In some cases, the one or more metabolites may be detected by non-invasive imaging, invasive imaging, or any combination thereof imaging. In some cases, the one or more metabolites and/or proteins may comprise a second set of metabolites and/or proteins configured to be detected by blood based, urine detection, or any combination thereof assays. In some cases, the treatment may comprise an oral available probiotic, an injection into the subject's tumor, an intravenous injection, or any combination thereof.

In some cases, the one or more reporter genes may enable the one or more microbes to release a tracer molecule detectable by imaging when the one or more microbes are in a particular tumor oxygen characteristic environment. In some instances, the tracer molecule may be a contrast agent for an imaging modality.

In some instances, the one or more microbes may comprise radioisotope-dosed microbes configured to be detected by positron emission tomography. In some cases, the radioisotope-dosed microbes may be configured to localize in a tumor oxygen characteristic environment dependent upon the oxygenation tolerance of the one or more microbes. In some instances, the oxygenation tolerance of the radioisotope-dosed microbes may be exploited to accumulate or localize the radioisotope marker during positron emission tomography. In some instances, the radioisotope-dosed microbes may therapeutically treat the tumor while providing a diagnostic function, thereby comprising theranostic characteristics, described elsewhere herein. In some cases, the radioisotope-dosed microbes may detect hypoxic tissues, such as poorly perfused organs, in a subject without cancer.

In some cases, the one or more engineered microbes comprising the theranostic may comprise a self-destruct feature, such as lysis, after therapeutically treating the cancer and/or not having detected a cancer in the subject.

In some cases, administering the one or more microbes to the subject may comprise an intravenous injection (IV), localized injection into and/or around the site of the tumor, intramuscular injections, or any combination thereof administrations.

In some cases, the one or more metabolites may indicate the prognosis of the cancer-bearing subject's disease-free survival, progression-free survival, disease-specific survival, overall survival, or any combination thereof.

### Administering Genetically Engineered Bacteria for Tumor Oxygen Characteristic Treatment

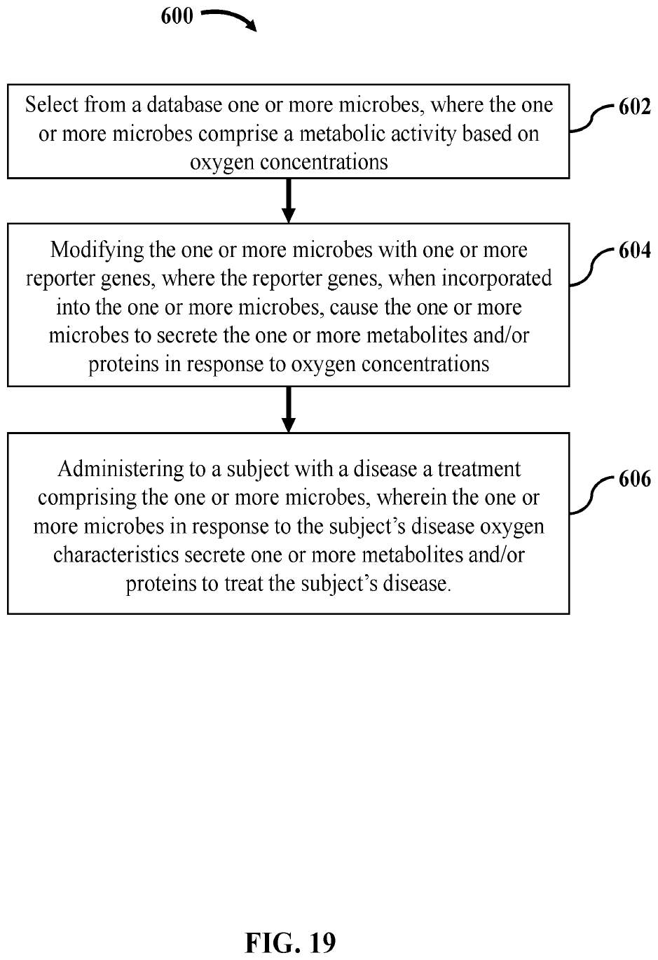

Aspects of the disclosure provided herein comprise a method of administering one or more microbes to determine treat a subject's disease 600, as seen in FIG. 19. In some cases, the method may comprise the following steps in any order: (a) selecting from a database one or more microbes, where the one or more microbes comprise a metabolic activity based on oxygen concentration 602; (b) modifying the one or more microbes with one or more reporter genes, when incorporated into the one or more microbes, cause the one or more microbes to secrete one or more metabolites and/or proteins in response to oxygen concentrations 604; and (c) administering to a subject with a disease a treatment comprising the one or more microbes, where the one or more microbes in response to the subject's disease oxygen characteristic secrete one or more metabolites and/or proteins to treat the subject's disease 606.

In some cases, the subject's disease may comprise cancer and/or a tumor. In some instances, the subject's cancer and/or tumor may comprise cancer of the breast, lung, bone, brain, pancreas, ovarian, colorectal, skin, or any combination thereof cancers. In some instances, the one or more microbes or their one or more metabolites and/or proteins may comprise anticancer properties. In some cases, the one or more metabolites and/or proteins may be detected by non-invasive imaging, invasive imaging, or any combination thereof imaging. In some cases, the one or more metabolites and/or proteins may comprise a second set of metabolites and/or proteins configured to be detected by blood based, urine detection, or any combination thereof assays. In some cases, the treatment may comprise an oral available probiotic, an injection into the subject's tumor, an intravenous injection, or any combination thereof.

In some cases, the one or more reporter genes may enable the one or more microbes to release a tracer molecule detectable by imaging when the one or more microbes are in a particular tumor oxygen characteristic environment. In some instances, the tracer molecule may be a contrast agent for an imaging modality.

In some instances, the one or more microbes may comprise radioisotope-dosed microbes configured to be detected by positron emission tomography. In some cases, the radioisotope-dosed microbes may be configured to localize in a tumor oxygen characteristic environment dependent upon the oxygenation tolerance of the one or more microbes. In some instances, the oxygenation tolerance of the radioisotope-dosed microbes may be exploited to accumulate or localize the radioisotope marker during positron emission tomography. In some instances, the radioisotope-dosed microbes may therapeutically treat the tumor while providing a diagnostic function, thereby comprising theranostic characteristics, described elsewhere herein. In some cases, the radioisotope-dosed microbes may detect hypoxic tissues, such as poorly perfused organs, in a subject without cancer.

In some cases, the one or more modified microbes may comprise a self-destruct feature, such as lysis, after therapeutically treating the cancer and/or not having detected a cancer in the subject.

In some cases, administering the one or more microbes to the subject may comprise an intravenous injection (IV), localized injection into and/or around the site of the tumor, intramuscular injections, or any combination thereof administrations.

In some cases, the one or more metabolites and/or proteins may indicate the prognosis of the cancer-bearing subject's disease-free survival, progression-free survival, disease-specific survival, overall survival, or any combination thereof.

### Using a Microbial Oxygen Characteristic Trained Predictive Model to Determine Treatment Outcome or Suggestion

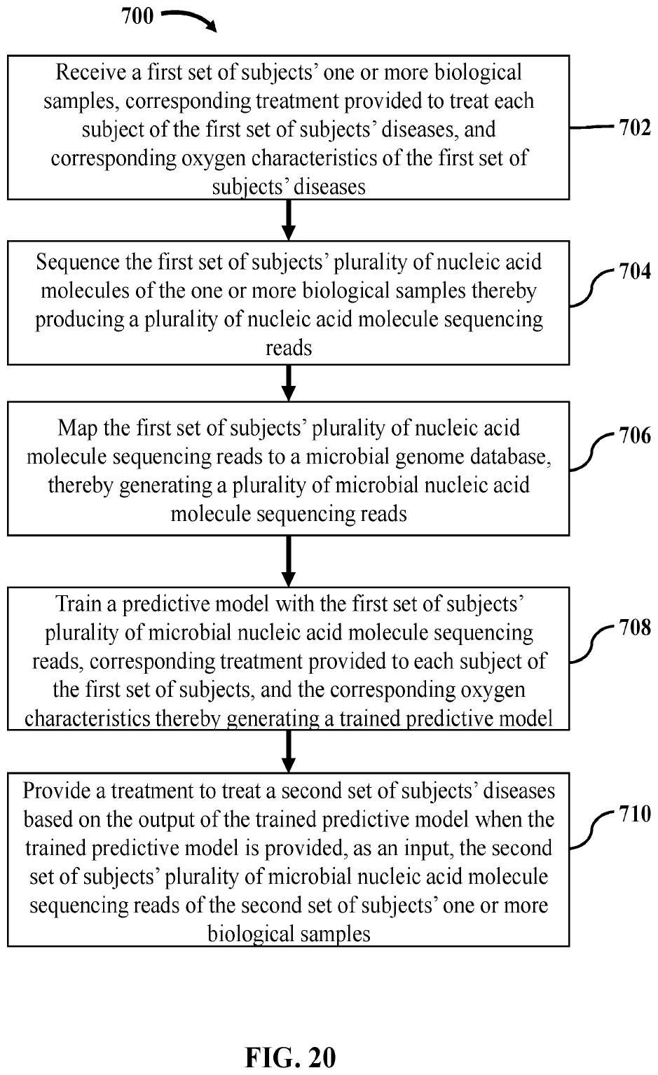

Aspects of the disclosure provided herein may comprise a method 700, as seen in FIG. 20, of providing a treatment to a set of subjects based on tumor oxygenation characteristics. In some cases, the method may comprise the following steps in any order: (a) receiving a first set of subjects' one or more biological samples and corresponding treatment provided to treat each subject of the first set of subjects' diseases 702; (b) sequencing the first set of subjects' plurality of nucleic acid molecules of the one or more biological samples thereby producing a plurality of nucleic acid molecule sequencing reads 704; (c) mapping the first set of subjects' plurality of nucleic acid molecule sequencing reads to a microbial genome database, thereby generating a plurality of microbial nucleic acid molecule sequencing reads 706; (d) training a predictive model with the first set of subjects' plurality of microbial nucleic acid molecule sequencing reads and corresponding treatment provided to each subject of the first set of subjects, thereby generating a trained predictive model 708; (e) providing a treatment to treat a second set of subjects' diseases based on the output of the trained predictive model when the trained predictive model is provided, as an input, the second set of subjects' plurality of microbial nucleic acid molecule sequencing reads of the second set of subjects' one or more biological samples 710. In some cases, the predictive model may be trained with the first set of subjects' plurality of microbial nucleic acid molecule sequencing reads, corresponding treatment provided to each subject of the first set of subjects, corresponding treatment outcome of each subject of the first set subjects, or any combination thereof. In some cases, the treatment may be configured to optimally treat the second set of subjects' disease. In some cases, the treatment may comprise an anti-angiogenic (e.g., VEGF inhibitors, bevacizumab), non-anti-angiogenic, or any combination thereof treatments. In some cases, the treatment may modify the tumor microbiome. In some instances, the first or second set of subjects' diseases may comprise cancer. In some cases, the trained predictive model may output a treatment recommendation, prognosis of providing the treatment recommendation, or any combination thereof.

In some cases, the first or second set of subjects' one or more biological samples may comprise a tissue biopsy, a liquid biopsy, or any combination thereof. In some cases, the tissue biopsy may comprise cancerous tissue, non-cancerous tissue, or any combination thereof. In some instances, the liquid biopsy may comprise whole blood, red blood cells, plasma, white blood cells, saliva, urine, tears, breast milk, or any combination thereof.

In some cases, the first or second set of subjects' plurality of microbial nucleic acid molecule sequencing reads may originate from bacterial aerobes, microaerophiles, anaerobes, facultative aerobes, facultative anaerobes, or any combination thereof. In some instances, the first or second set of subjects' plurality of nucleic acid molecule comprise microbial DNA, microbial RNA, epigenetic markers on microbial DNA, epigenic markers on microbial RNA, human RNA, human DNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

In some instances, the method may further comprise decontaminating the first or second set of subjects' plurality of microbial nucleic acid molecule sequencing reads thereby producing a plurality of decontaminated microbial nucleic acid molecule sequencing reads. In some cases, the decontaminated microbial nucleic acid molecule sequencing reads may then be used to train the predictive model, where the first set of subjects' plurality of decontaminated microbial nucleic acid molecule sequencing reads and corresponding treatment may be used as training and/or validation data. In some cases, decontamination may be conducted in silico, using experimental contamination controls, or any combination thereof.

The decontamination of the first or second set of subjects' plurality of nucleic acid molecule sequencing reads may increase the accuracy of the trained predictive models described elsewhere herein. In some cases, decontaminating may be conducted in silico, with experimental contamination controls, using limit of quantification filtering, or any combination thereof method of decontamination. In some cases in silico decontamination may comprise comparing individual microbial abundances across one or more biological samples of varying analyte concentrations. The one or more contaminate microbes may be identified by a fractional abundance of microbial reads that are inversely proportional to the analyte concentrations of one or more biological samples. For example, at lower analyte concentrations, the contaminate microbes will have a higher fractional read abundance compared to the overall abundance of the microbial nucleic acid molecules. In some instances, such a decontamination method may comprise the steps of: (i) measuring a plurality of analyte concentrations from the one or more biological samples of a subject; (ii) sequencing the plurality of nucleic acid molecules at the plurality of dilutions to generate a plurality of nucleic acid molecule sequences; (iii) mapping the plurality of nucleic acid molecule sequencing reads to a microbial genome database thereby generating a plurality of microbial nucleic acid molecule reads of the plurality of dilutions; (iv) identifying contaminate microbes from the plurality of microbial nucleic acid molecule reads where the contaminate microbes are present with a fractional abundance that is inverse proportional to the plurality of dilutions across one or more biological samples; and (v) removing the contaminate microbes from the microbial nucleic acid molecule reads prior to step (d) 708.

In some instances, decontamination by experimental contamination controls may comprise identifying the presence of microbial nucleic acid molecules within one or more negative control samples (e.g., empty sample collection vessels, vials, dishes, sealable containers, swabs, vials only of reagents, etc.) that may be removed from the plurality of microbial nucleic acid molecules prior to step (d). In some cases, microbes and their microbial nucleic acid molecules are removed if identified in proportionately more negative control samples than biological samples. In some cases, microbes and their microbial nucleic acid molecules are removed on the basis of a statistical test, such as a Fisher exact test, that describes differences in presence proportionality of the microbial nucleic acid molecules between negative controls and biological samples. In some cases, a method of decontamination by experimental contamination controls may comprise the steps of: (i) obtaining one or more negative control vessels or chambers or reagents used to transport and/or store and/or process the one or more biological samples; (ii) sequencing nucleic acid molecules of the one or more negative control vessels, thereby generating a plurality of negative control sequencing reads; (iii) mapping the plurality of negative control sequencing reads to a microbial genome database thereby generating a plurality of microbial nucleic acid molecule reads; and (iv) removing the plurality of negative control microbial nucleic acid molecule reads from the microbial nucleic reads of the one or more biological samples prior to step (d).

In some instances, the trained predictive model may comprise a machine learning model. In some cases, the trained predictive model may comprise a regularized machine learning model. In some instances, the trained predictive model may comprise one or more machine learning models, an ensemble of machine learning models, or any combination thereof. In some instances, the trained predictive model may comprise a gradient boosting machine, neural network, support vector machine, k-means, classification trees, random forest, regression, or any combination thereof machine learning model. In some cases, the machine learning model may comprise an accuracy of at least 60%, at least 70%, at least 80%, at least 85%, at least 90%, at least 95%, at least 97%, or at least 99%.

### Spatial Oxygen Characterization of Tissue Sections

Aspects of the disclosure provided herein may comprise a method of determining the spatial oxygen characteristic of a subject's tissue based on microbial analytes. In some cases, the method may comprise the steps of: (a) receiving one or more tissues of a subject; (b) isolating one or more microbial analytes from one or more spatially distinct regions of the one or more tissue sections; and (c) determining a spatial oxygen characteristic of a subject's tissue section identifying a plurality of microbes based on one or more microbial analytes of the one or more spatially distinct regions. In some cases, the tissues of the subject may comprise a tissue section, tissue biopsy, or any combination thereof. In some instances, the microbial analytes may comprise microbial DNA, RNA, proteins, metabolites, or any combination thereof.

In some cases, a list of a plurality of microbes that produce the one or more microbial analytes used to determine the spatial oxygen characteristic of a subject's tissue section and/or to spatially assay tumor hypoxia may be found in Table 1. Additionally, Table 1 describes the known oxygen tolerance, genus and/or species classification of the microbe, and/or genetic target for the microbe.

TABLE 1
Microbe Targets for Spatial Tissue Oxygen Characterization

| | Oxygen | | Target |
|---|---|---|---|
| Microbe Name | Tolerance | Resolution | Type |
| Bacteroides dorei | anaerobe | Species | 16S |
| | | | rRNA |
| Bacteroides vulgatus | anaerobe | Species | 16S |
| | | | rRNA |
| Bacteroides uniformis | anaerobe | Species | rRNA |
| | | | operon |
| Bacteroides fragilis | anaerobe | Species | rRNA |
| | | | operon |
| Gardnerella vaginalis | anaerobe | Species | 16S |
| | | | rRNA |
| Lactobacillus iners | anaerobe | Species | 16S |
| | | | rRNA |
| Prevotella timonensis | anaerobe | Species | 16S |
| | | | rRNA |
| Veillonella parvula | anaerobe | Species | rRNA |
| | | | operon |
| Fusobacterium nucleatum | anaerobe | Species | rRNA |
| | | | operon |
| Faecalibacterium prausnitzii | anaerobe | Species | rRNA |
| | | | operon |
| Enterococcus faecalis | anaerobe | Species | rRNA |
| | | | operon |
| Akkermansia muciniphila | anaerobe | Species | 16S |
| | | | rRNA |
| Clostridium perfringens | anaerobe | Species | rRNA |
| | | | operon |
| Rothia mucilaginosa | microaerophile | Species | 16S |
| | | | rRNA |
| Pseudomonas aeruginosa | aerobe | Species | dacC |
| | | | gene |
| Corynebacterium accolens | microaerophile | Species | rpoB |
| | | | gene |
| Roseomonas mucosa | aerobe | Species | 16S |
| | | | rRNA |
| Ruminococcus gnavus/lactaris | anaerobe | Mixed | 16S |
| | | species | rRNA |
| Mycobacterium | aerobe | Mixed | 16S |
| pneumoniae/genitalium | | species | rRNA |
| Streptococcus | microaerophile | Mixed | 23S |
| periodonticum/gordonii/anginosus | | species | rRNA |
| Klebsiella oxytoca/michiganensis | aerobe | Mixed | 16S |
| | | species | rRNA |
| Bifidobacterium | anaerobe | Mixed | 16S |
| animalis/pseudolongum | | species | rRNA |
| Porphyromonas gingivalis/gulae | anaerobe | Mixed | 16S |
| | | species | rRNA |
| Porphyromonas | anaerobe | Mixed | 16S |
| uenonis/asaccharolytica | | species | rRNA |
| Acidovorax [genus] | aerobe | Genus | 16S |
| | | | rRNA |
| Bacteroides [genus] | anaerobe | Genus | 16S |
| | | | rRNA |
| Campylobacter [genus] | anaerobe | Genus | 16S |
| | | | rRNA |
| Corynebacterium [genus] | microaerophile | Genus | 16S |
| | | | rRNA |
| Fusobacterium [genus] | anaerobe | Genus | 16S |
| | | | rRNA |
| Gardnerella [genus] | anaerobe | Genus | 16S |
| | | | rRNA |
| Lactobacillus [genus] | anaerobe | Genus | 16S |
| | | | rRNA |
| Mycobacterium [genus] | aerobe | Genus | 16S |
| | | | rRNA |
| Prevotella [genus] | anaerobe | Genus | 16S |
| | | | rRNA |
| Pseudomonas [genus] | aerobe | Genus | 16S |
| | | | rRNA |
| Rothia [genus] | aerobe or | Genus | 16S |
| | microaerophile | | rRNA |
| Staphylococcus [genus] | aerobe | Genus | 16S |
| | | | rRNA |
| Streptococcus [genus] | microaerophile | Genus | rRNA |
| | | | operon |
| Veillonella [genus] | anaerobe | Genus | 16S |
| | | | rRNA |
| Neisseria [genus] | aerobe | Genus | 16S |
| | | | rRNA |
| Eubacteria | N/A | Pan- | 16S |
| | | bacteria | rRNA |
| Scrambled_Eubacteria | N/A | Control | 16S |
| | | | rRNA |
| Malassezia globosa | prefers aerobic | Fungi | 28S |
| | environ. | | rRNA |
| Malassezia restricta | prefers aerobic | Fungi | 28S |
| | environ. | | rRNA |
| Candida albicans | prefers aerobic | Fungi | 28S |
| | environ. | | rRNA |

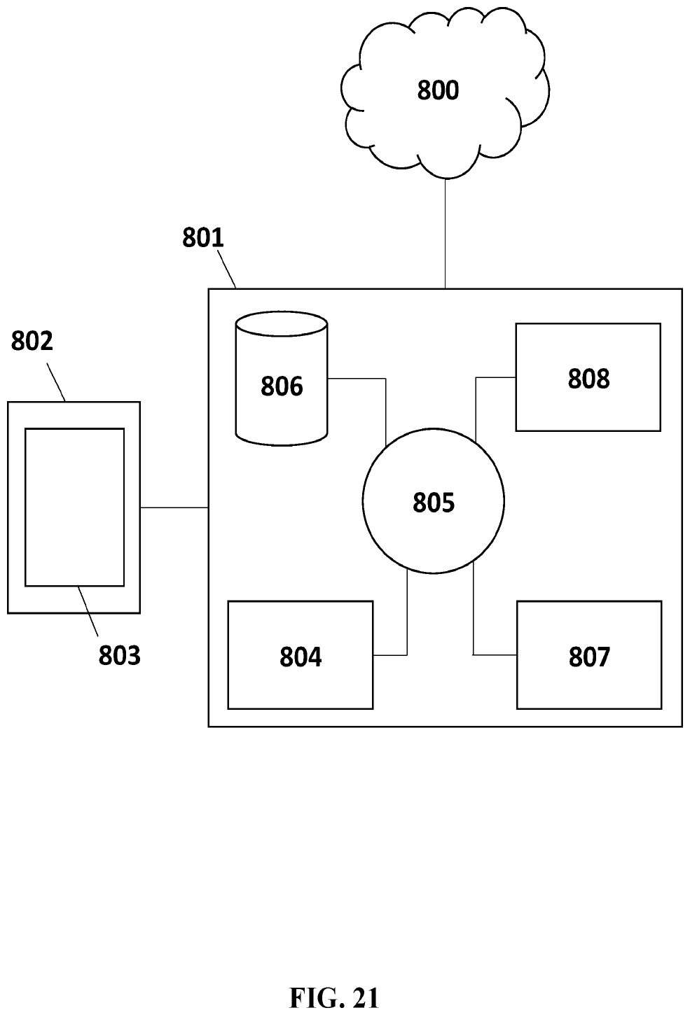

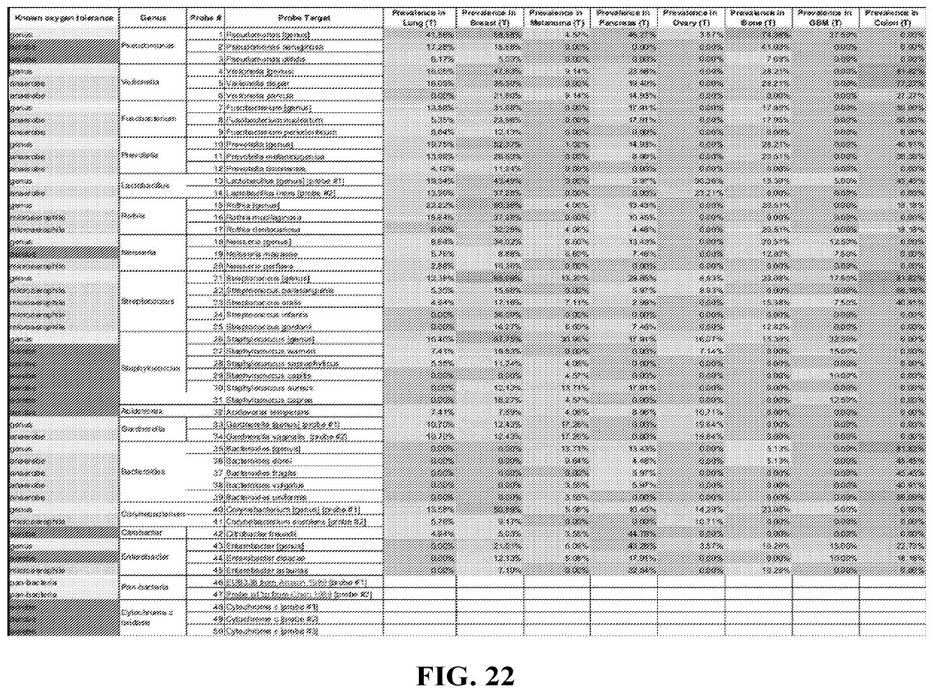

In some cases, a list of a plurality of microbes based on one or more microbial analytes may be used to determine the spatial oxygen characteristic of a subject's tissue section be found in FIG. 22. FIG. 22 particularly shows the percent observed prevalence for a plurality of decontaminated microbes with known oxygen tolerance for a particular cancer type (e.g., lung, breast, melanoma, pancreases, ovary, bone, GBM, and colorectal).

### II. Systems

FIG. 21 shows a computer system 801 suitable for implementing and/or training the models and/or predictive models described herein. The computer system 801 may process various aspects of information, data, and/or samples of the present disclosure, such as, for example, subjects' biological samples. The computer systems may assist, facilitate, and/or conduct the processes of extracting the plurality of nucleic acid molecule samples from the one or more biological samples, described elsewhere herein. In some cases, the computer systems may assist, facilitate, and/or conduct the process of accessing, downloading, categorizing, and further processing a plurality of sequencing reads of one or more subjects' biological samples stored in a database, as described elsewhere herein. The computer system 801 may be an electronic device. The electronic device may be a mobile electronic device.

The computer system 801 may comprise a central processing unit (CPU, also “processor” and “computer processor” herein) 805, which may be a single core or multi core processor, or a plurality of processor for parallel processing. The computer system 801 may further comprise memory or memory locations 804 (e.g., random-access memory, read-only memory, flash memory), electronic storage unit 806 (e.g., hard disk), communications interface 808 (e.g., network adapter) for communicating with one or more other devices, and peripheral devices 807, such as cache, other memory, data storage and/or electronic display adapters. The memory 804, storage unit 806, interface 808, and peripheral devices 807 are in communication with the CPU 805 through a communication bus (solid lines), such as a motherboard. The storage unit 806 may be a data storage unit (or a data repository) for storing data. The computer system 801 may be operatively coupled to a computer network (“network”) 800 with the aid of the communication interface 808. The network 800 may be the Internet, an internet and/or extranet, or an intranet and/or extranet that is in communication with the Internet. The network 800 may, in some cases, be a telecommunication and/or data network. The network 800 may include one or more computer servers, which may enable distributed computing, such as cloud computing. The network 800, in some cases, with the aid of the computer system 801, may implement a peer-to-peer network, which may enable devices coupled to the computer system 801 to behave as a client or a server.

The CPU 805 may execute a sequence of machine-readable instructions, which may be embodied in a program or software. The instructions may be directed to the CPU 805, which may subsequently program or otherwise configured the CPU 805 to implement methods of the present disclosure. Examples of operations performed by the CPU 805 may include fetch, decode, execute, and writeback.

The CPU 805 may be part of a circuit, such as an integrated circuit. One or more other components of the system 801 may be included in the circuit. In some cases, the circuit is an application specific integrated circuit (ASIC).

The storage unit 806 may store files, such as drivers, libraries, and saved programs. The storage unit 806 may store one or more sequencing reads of one or more subjects' biological sample, cancer type if present, treatment administered to treat the cancer, treatment efficacy of the treatment administered, HIPPA protected meta health data (e.g., age, sex, gender, other diseases, or conditions, etc.), or any combination thereof. The computer system 801, in some cases may include one or more additional data storage units that are external to the computer system 801, such as located on a remote server and/or cloud, that is in communication with the computer system 801 through an intranet or the internet.

Methods as described herein may be implemented by way of machine (e.g., computer processor) executable code stored on an electronic storage location of the computer device 801, such as, for example, on the memory 804 or electronic storage unit 806. The machine executable or machine-readable code may be provided in the form of software. During use, the code may be executed by the processor 805. In some instances, the code may be retrieved from the storage unit 806 and stored on the memory 804 for ready access by the processor 805. In some instances, the electronic storage unit 806 may be precluded, and machine-executable instructions are stored on memory 804.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 801, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media include dynamic memory, such as main memory of such a computer platform. Tangible transmission media includes coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 802 that comprises a user interface (UI) 803 for viewing one or more subjects' therapeutic treatment recommendation outputted by a trained predictive model, determination of a presence or lack thereof cancer for one or more subjects, determination of an oxygen characteristic for one or more biological samples, determination of one or more microbes suitable for engineering based on a database of intratumoral microbes and their oxygen tolerances, one or more feature characteristics of the one or more sequence nucleic sequencing reads processed by the system (e.g., microbial taxa and/or functional pathways) of one or more groups of subjects, training results of the predictive models described elsewhere herein, or any combination thereof operations. Examples of UI's include, without limitation, a graphical user interface (GUI) and web-based user interface.

Methods and systems of the present disclosure can be implemented by way of one or more predictive models, or an ensemble of predictive models, described elsewhere herein, and with instructions provided with one or more processors as disclosed herein. A predictive model may be implemented by way of software upon execution by the central processing unit 805.

The predictive model(s), described elsewhere herein, may comprise one or more machine learning algorithms and/or models and may have an accuracy greater than about 60%, 70%, 80%, 85%, 90%, 95%, or 99%. The machine learning algorithm may have a positive predictive value greater than about 60%, 70%, 75%, 80%, 85%, 90%, 95%, or 99%. The machine learning algorithm may have a negative predictive value greater than about 60%, 70%, 80%, 90%, 95%, or 99%.

One or more machine learning algorithms may be used to construct the machine learning model, as described elsewhere herein, such as support vector machines that deploy stepwise backwards parameter selection and/or graphical models, both of which may have advantages of inferring interactions between parameters and/or features of the one or more microbial nucleic acid molecule sequences, treatments provided, treatment efficacy, subject clinical metadata, or any combination thereof data analyzed by methods described herein. For example, machine learning algorithms or other statistical algorithms may be used such as alternating decision trees (ADTree), decision stumps, functional trees (FT), logistic model trees (LMT), logistic regression, random forests (rf), receiver operational characteristic curves (ROC), linear regression, extreme gradient boosting (xgb), classification and regression trees, support vector machines (SVM), generalized additive model using splines (e.g., gamSpline), glmnet, multivariate adaptive regression splint (earth), neural network, k-means clustering, ridge regression, Bayesian ridge regression (i.e., bridge regression), or any machine learning algorithm or statistical algorithm known in the art. One or more algorithms may be used together to generate an ensemble method, wherein the ensemble method may be optimized using a machine learning ensemble meta-algorithm such as boosting (e.g., AdaBoost, LPBoost, TotalBoost, BrownBoost, MadaBoost, LogitBoost, etc.) to reduce bias and/or variance. Once a machine learning model is derived from the training data, the model may be use as a prediction tool to determine of a presence or lack thereof cancer for one or more subjects, determinate of an oxygen characteristic for one or more biological samples, determination of one or more microbes suitable for engineering based on a database of intratumoral microbes and their oxygen tolerances, and/or to recommend an optimal treatment to a subject to treat said subject's disease. Machine learning analyses may be performed using one or more of many programming languages and platforms known in the art, such as R, Weka, Python, and/or MATLAB, for example.

Aspects of the disclosure provided herein, may comprise a system configured to output an estimate of tumor oxygenation of a subject. In some cases, the method may comprise: (a) one or more processors; and (b) a non-transient computer readable storage medium including software, where the software comprises executable instructions that, as a result of execution, cause the one or more processors of the computer system to: (i) receive one or more biological samples of a subject; (ii) sequence a plurality of nucleic acid molecules of the one or more biological samples thereby generating a plurality of nucleic acid molecule sequencing reads; (iii) map the plurality of nucleic sequencing reads to a microbial genome database thereby generating a plurality of microbial nucleic acid molecule reads; and (iv) output an estimate of tumor oxygenation of the subject as an output of a trained predictive model when the plurality of microbial nucleic acid molecule reads are provided as an input to the trained predictive model. In some cases, the instructions may further comprise decontaminating the plurality of nucleic acid molecule sequencing reads thereby producing a plurality of decontaminated nucleic acid molecule sequencing reads. In some instances, decontamination may be conducted in silico, using experimental contamination controls, or any combination thereof. In some cases, the subject's tumor may comprise a breast, lung, bone, brain, pancreas, ovarian, colorectal, skin, or any combination thereof tumors. In some cases, the subject's tumor may comprise cancerous cells, cells that have metastasized, and/or cells that are benign.

In some instances, the one or more biological samples may comprise a tissue biopsy, a liquid biopsy, or any combination thereof. In some cases, the tissue biopsy may comprise cancerous tissue, non-cancerous tissue, or any combination thereof. In some instances, the liquid biopsy comprises whole blood, red blood cells, plasma, white blood cells, saliva, urine tears, breast milk, or any combination thereof.

In some cases, the plurality of microbial nucleic acid molecules may originate from bacterial aerobes, microaerophiles, anaerobes, facultative aerobes, facultative anaerobes, or any combination thereof. In some instances, the plurality of nucleic acid molecules comprise microbial DNA, microbial RNA, epigenetic markers on microbial DNA, epigenetic markers on microbial RNA, human RNA, human DNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

In some instances, the trained predictive model may comprise one or more machine learning models, an ensemble of machine learning models, or any combination thereof. In some cases, the trained predictive model may comprise a regularized machine learning model. In some instances, the trained predictive model may comprise a gradient boosting machine, neural network, support vector machine, k-means, classification trees, random forest, regression, or any combination thereof machine learning models. In some cases, the trained predictive model is trained with microbial DNA, microbial RNA, epigenetic marks on microbial DNA, epigenetic marks on microbial RNA, cell-free microbial RNA, cell-free microbial DNA, non-microbial DNA, non-microbial RNA, epigenetic marks on non-microbial DNA, epigenetic marks on non-microbial RNA, non-microbial cell free DNA, non-microbial cell free RNA, or any combination thereof.

Although the methods and systems described herein may a series of steps, a person of ordinary skill in the art will recognize many variations based on the teaching described herein. Steps of the methods provided herein may be completed in a different order. Steps may be added or deleted. Some of the steps may comprise sub-steps. Many of the steps may be repeated as often as is beneficial to the platform. Additionally a particular embodiments may be described herein, where such embodiments may comprise methods that combine steps of one or more methods described herein.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, diagnostic kit, a pharmaceutical composition, and/or a method that “comprises” a list of elements (e.g., components, features, or steps) is not necessarily limited to only those elements (or components or steps) but may include other elements (or components or steps) not expressly listed or inherent to the diagnostic kit, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. It is understood that aspects and embodiments of the invention described herein include “consisting” and/or “consisting essentially of” aspects and embodiments.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight, or length that varies by as much as 15%, 10%, 9%, 8%, 7%, 6%, 5%, 4%, 3%, 2% or 1% to a reference quantity, level, value, number, frequency, percentage, dimension, size, amount, weight, or length. In various embodiments, the term “about” or “approximately” refers a range of quantity, level, value, number, frequency, percentage, dimension, size, amount, weight, or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight, or length.

In embodiments, the invention provides methods for the detection of biologic markers indicative of the presence of a cancer. In embodiments, the invention comprises methods for the diagnosis of cancer.

“Amplification” refers to any known procedure for obtaining multiple copies of a target nucleic acid molecule or its complement, or fragments thereof. The multiple copies may be referred to as amplicons or amplification products. Amplification, in the context of fragments, refers to production of an amplified nucleic acid molecule that contains less than the complete target nucleic acid molecule or its complement, e.g., produced by using an amplification oligonucleotide that hybridizes to, and initiates polymerization from, an internal position of the target nucleic acid molecule. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid molecule or a region thereof. Amplicons can be double stranded or single stranded and can include DNA, RNA, or both. Methods for generating amplicons are known to those skilled in the art.

“Complementary” or “complement thereof” means that a contiguous nucleic acid molecule base sequence is capable of hybridizing to another base sequence by standard base pairing (hydrogen bonding) between a series of complementary bases. Complementary sequences may be completely complementary (i.e. no mismatches in the nucleic acid molecule duplex) at each position in an oligomer sequence relative to its target sequence by using standard base pairing (e.g., G: C, A: T or A: U pairing) or sequences may contain one or more positions that are not complementary by base pairing (e.g., there exists at least one mismatch or unmatched base in the nucleic acid molecule duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid molecule sequence configuration of a referenced oligonucleotide. For example, a primer that is configured to generate a specified amplicon from a target nucleic acid molecule has a nucleic acid molecule sequence that hybridizes to the target nucleic acid molecule or a region thereof and can be used in an amplification reaction to generate the amplicon. Also as an example, an oligonucleotide that is configured to specifically hybridize to a target nucleic acid molecule or a region thereof has a nucleic acid molecule sequence that specifically hybridizes to the referenced sequence under stringent hybridization conditions.

“Downstream” means further along a nucleic acid molecule sequence in the direction of sequence transcription or read out.

“Upstream” means further along a nucleic acid molecule sequence in the direction opposite to the direction of sequence transcription or read out.

“Polymerase chain reaction” (PCR) generally refers to a process that uses multiple cycles of nucleic acid molecule denaturation, annealing of primer pairs to opposite strands (forward and reverse), and primer extension to exponentially increase copy numbers of a target nucleic acid molecule sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Position” refers to a particular amino acid or amino acids in a nucleic acid molecule sequence.

“Primer” refers to an enzymatically extendable oligonucleotide, generally with a defined sequence that is designed to hybridize in an antiparallel manner with a complementary, primer-specific portion of a target nucleic acid molecule. A primer can initiate the polymerization of nucleotides in a template-dependent manner to yield a nucleic acid molecule that is complementary to the target nucleic acid molecule when placed under suitable nucleic acid molecule synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid molecule, or in an amplified nucleic acid molecule, under conditions that promote hybridization to allow selective detection of the target sequence.

A primer nucleic acid molecule can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acid molecules can also be used as probe nucleic acid molecules.

“Region” refers to a portion of a nucleic acid molecule wherein said portion is smaller than the entire nucleic acid molecule.

“Region of interest” refers to a specific sequence of a target nucleic acid molecule that includes all codon positions having at least one single nucleotide substitution mutation associated with a genotype and/or subtype that are to be amplified and detected, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptase also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DNA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in 5′-to-3′ direction beginning at a specific position just downstream of the promoter. Examples of transcriptase are the DNA-dependent RNA polymerase from E. coli and bacteriophages T7, T3, and SP6.

A “sequence” of a nucleic acid molecule refers to the order and identity of nucleotides in the nucleic acid molecule. A sequence is typically read in 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997)” “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

“Fragment” refers to a piece of contiguous nucleic acid molecule that contains fewer nucleotides than the complete nucleic acid molecule.

“Hybridization,” “annealing,” “selectively bind,” or “selective binding” refers to the base-pairing interaction of one nucleic acid molecule with another nucleic acid molecule (typically an antiparallel nucleic acid molecule) that results in formation of a duplex or other higher-ordered structure (i.e. a hybridization complex). The primary interaction between the antiparallel nucleic acid molecules is typically base specific, e.g., A/T and G/C. It is not a requirement that two nucleic acid molecules have 100% complementarity over their full length to achieve hybridization. Nucleic acid molecules hybridize due to a variety of well characterized physio-chemical forces, such as hydrogen bonding, solvent exclusion, base stacking, and the like. An extensive guide to the hybridization of nucleic acid molecules is found in Tijssen (1993) Laboratory Techniques in Biochemistry and Molecular Biology-Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable diluent or excipient” or “pharmaceutically acceptable carrier” refers to an excipient, diluent, preservative, solubilizer, emulsifier, adjuvant, and/or vehicle with which an NK cell of the disclosure, is administered. Such carriers may be sterile liquids, such as water and oils, including those of petroleum, animal, vegetable, or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable diluent or excipient” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable diluents or excipients, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates, and buffering agents or sterile, pyrogen-free, water. Exemplary administration forms may include solutions or suspensions in sterile aqueous solutions, for example, aqueous propylene glycol or dextrose solutions. Such dosage forms can be suitably buffered, if desired.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently, or sequentially with no specific time limits, wherein such administration provides therapeutically effective levels of the two compounds in the body of the patient. The latter also applies to cocktail therapy, e.g., the administration of three or more active ingredients.

In an aspect, the disclosure provides a method of diagnosing and treating a disease or disorder, such as cancer, in a subject in need thereof, comprising administering the pharmaceutical composition of the disclosure to the subject. In some embodiments, the disease or disorder is a malignancy. In some embodiments, the malignancy comprises a tumor-associated marker.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits cancer that can be treated with the compositions and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject. As used herein, the term “amount” refers to “an amount effective” or “an effective amount” of a pharmaceutical composition, including a cell, to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse, or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

The pharmaceutical compositions of the disclosure may be administered in a number of ways depending upon whether local or systemic treatment is desired. The pharmaceutical compositions are typically suitable for parenteral administration, wherein administration includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intranasal, intratracheal, intraarterial, intrathecal, intraventricular, intraurethral, intracranial, intratumoral, intraocular, intradermal, intrasynovial injection or infusions, intra-tumoral; and kidney dialytic infusion techniques.

The section headings used herein are for organizational purposes only and are not to be construed as limiting the subject matter described.

### IV. EXAMPLES

The following examples are included for illustrative purposes only and are not intended to limit the scope of the invention. Various further aspects and embodiments of the disclosure will be apparent to one skilled in the art in view of the above non-limiting examples and description of the invention.

The results of the various examples below show to one of ordinary skill in the art that: (i) bacterial diversity correlates significantly with tumor hypoxia, analogous to how bacterial diversity correlates with environment (e.g., sediment) oxygen deficits (Hoshino et al. 2020. PNAS); (ii) microbial community compositions can distinguish hypoxic versus normoxic tumors; (iii) microbial functions can distinguish hypoxic versus normoxic tumors; and (iv) bacteria of multiple distinct oxygen tolerances co-exist within individual tumors, as observed even with extremely stringent decontamination. Collectively, the methods and data herein show that microbial compositions and their functions are influenced by and can predict tumor hypoxia. Moreover, with strong correlations between tumor hypoxia and patient survival, response to therapy, and tumor aggressiveness (Walsh et al. 2014. Antioxid. Redox. Signal.), it has become clear that microbial compositions and functions within tumors may serve as prognostic biomarkers. Moreover, since these microbial biomarkers likely leak into circulation (e.g., plasma; Poore et al. 2020. Nature), their associations with intratumoral hypoxia may be captured using minimally invasive, liquid biopsy-derived prognostic tests.

### Example 1: Tumor Microbiome Hypoxia Determination

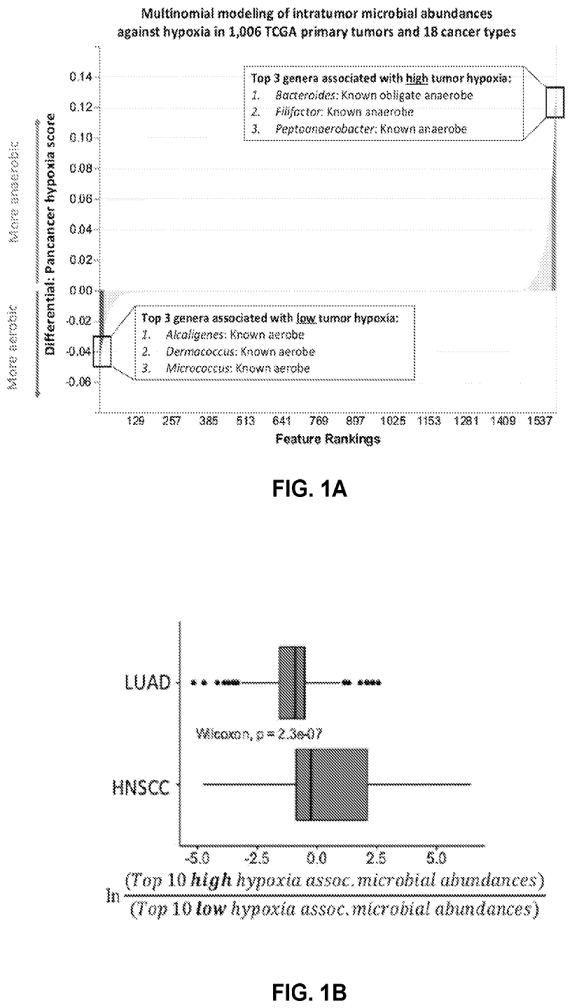

Multinomial regression coefficients were calculated and ranked for microbes derived from 1,006 primary tumor samples of 18 cancer types alongside their matched tumor hypoxia values (“scores”) to identify which microbes were most and least associated with hypoxia. Firstly, nucleic acid sequences of these 1,006 primary tumor samples were determined and aligned against the human genome. The resulting sequencing reads that did not align to the human genome were extracted and mapped to a database of microbial genomes to produce a table of microbial abundances. The table of microbial abundances were then summarized at the genus level and matched to estimated tumor hypoxia scores from the same tumors. The tumor hypoxia scores were derived from host RNA metagene expression (Buffa et al. 2010. British Journal of Cancer). Multinomial regression modeling was then utilized to infer which microbes were most and least associated with the matched hypoxic score. The multinomial modeling provided coefficient estimates for each microbe indicating the direction and strength of the association with hypoxia or normoxia for each genus. The more negative the coefficients, the more the genus level bacteria were associated with normoxia; conversely, the more positive coefficients were associated with hypoxia. These genus-ranked coefficients were then plotted from lowest to highest coefficient value, as seen in FIG. 1A. Additionally, the top ten genera most associated with normoxia (i.e., negative coefficient genus level bacteria) were colored in blue, and the top ten genera most associated with hypoxia (i.e., positive coefficients) were colored red, as can be seen in FIG. 1A. The figure inset of FIG. 1A also shows the top three normoxic and hypoxic-associated genera along with their oxygen tolerances.

The method outlined in Example 1 was further utilized to validate the discriminatory nature of the hypoxia score in differentiating tumors of different cancer types. Briefly, the initial dataset of 1,006 primary tumor samples were narrowed to lung adenocarcinoma (LUAD) and head and neck squamous cell carcinoma (HNSCC), as these two cancer types are known to have different average hypoxic levels, with HNSCC being more hypoxic on average than LUAD (Bhandari et al. 2019. Nature Genetics). Specifically, the abundances of the top ten genera associated with hypoxia were summed, followed by summing the abundances of the top ten genera associated with normoxia, followed by calculating the ratio thereof and taking the natural logarithm (equation shown symbolically on x-axis of FIG. 1B). The results of such ratios were plotted, statistically tested between LUAD and HNSCC using a Wilcoxon rank-sum test and are shown in FIG. 1B. From FIG. 1B, it can be seen that the methods outlined above are able to show significant differentiation between the two cancer types (p=2.3×10⁻⁷).

### Example 2 Validating Association Between Microbial Data and Hypoxia

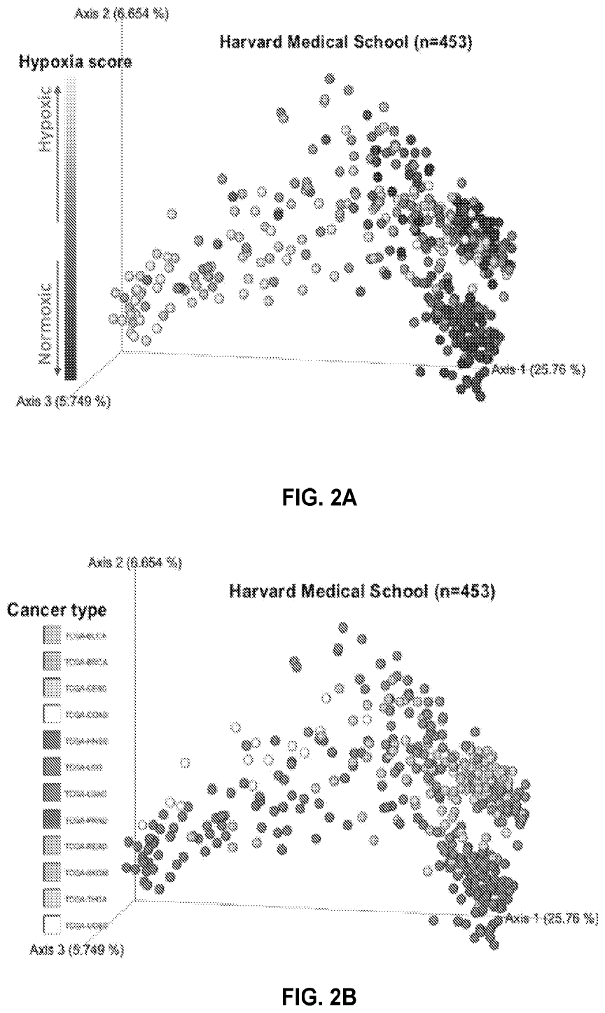

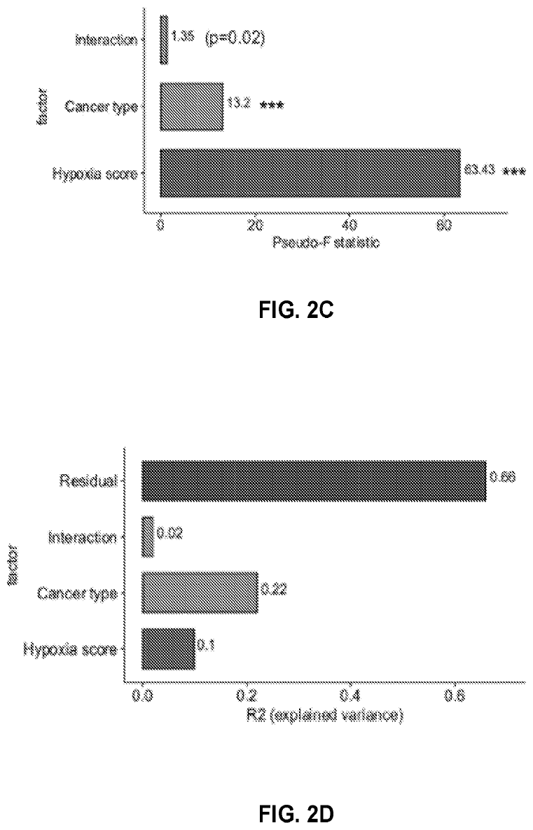

It has been previously established that microbial abundances of samples sequenced in the cancer genome atlas database (TCGA) may be affected by center batch effects (e.g. location of a sequencing center, the sequencing machine utilized, or analyte type considerations of DNA or RNA), at times accounting for substantial variance in the dataset. To preclude batch effects or batch effect correction as a potential cofounder in driving an association between microbial data and hypoxia, a selection of a limited dataset comprising a single ‘batch’ (a single sequencing center, sequencing machine type, and analyte type [DNA]) was made and analyzed. For purposes of this experiment, data from Harvard Medical School using an Illumina HiSeq sequencing machine analyzing DNA was used. In total, 453 treatment-naïve tumor samples, comprising the entirety of Harvard Medical School dataset, were available with matching RNA-Seq data in order to determine their matching hypoxia scores using hypoxic metagene expression (Buffa et al. 2010. British Journal of Cancer). The dataset was composed of the following cancer types at their respective quantities: 50 bladder (BLCA); 8 breast (BRCA); 2 cervical (CESC); 15 colorectal adenocarcinoma (COAD); 84 head and neck squamous cell carcinoma (HNSC); 26 lower grade glioma (LGG); 106 lung adenocarcinoma (LUAD); 70 prostate adenocarcinoma (PRAD); 1 rectal adenocarcinoma (READ); 14 melanoma (SKCM); 72 thyroid carcinoma (THCA); and 5 uterine corpus endometrial carcinoma (UCEC). Distance data for each sample among the 453 tumor samples were determined using unweighted UniFrac measurement after rarefying to 100,00 reads/sample. Next, principal coordinate analysis (PCoA) was conducted using the UniFrac distance data, and the samples were colored either by hypoxia score or cancer type, as plotted in FIGS. 2A-2B. FIG. 2A shows the results of the PCoA with samples colored by corresponding hypoxia score, as determined by RNA metagene expression. FIG. 2B shows the same results of the PCoA, instead coloring each sample by cancer type.

The distance data in FIGS. 2A-2B was then used in a permutational multivariate analysis of variance (PERMANOVA) test that included cancer type as a categorical variable, hypoxia as a continuous variable, and an interaction term that combined the two. The results of the PERMANOVA analysis can be seen in FIGS. 2C and 2D. FIG. 2C shows that the hypoxia score explains the clustering of hypoxic and normoxic samples, as revealed by the pseudo-F statistic. The asterisks denote significance (p≤0.001). The interaction term shown in FIGS. 2C-2D describes the grouping interaction between cancer type and hypoxia. As can be seen in FIG. 2C the interaction term is substantially weaker (˜47 times weaker pseudo-F statistic) than the hypoxia score itself, strongly suggesting that the hypoxia-driven variance is a pan-cancer effect. The residual shown in FIG. 2D indicates the amount of variance in the data that is not accounted for by cancer, type, hypoxia, or the interaction of cancer type and hypoxia. The similarity of cancer type and hypoxia score variance in FIG. 2D indicates that the hypoxia score explains a similar magnitude (10-20%) of variance as cancer type, suggesting that tumor hypoxia significantly modulates microbial abundances within tumors.

### Example 3: Comparing Weighted and Unweighted UniFrac Distance Calculations

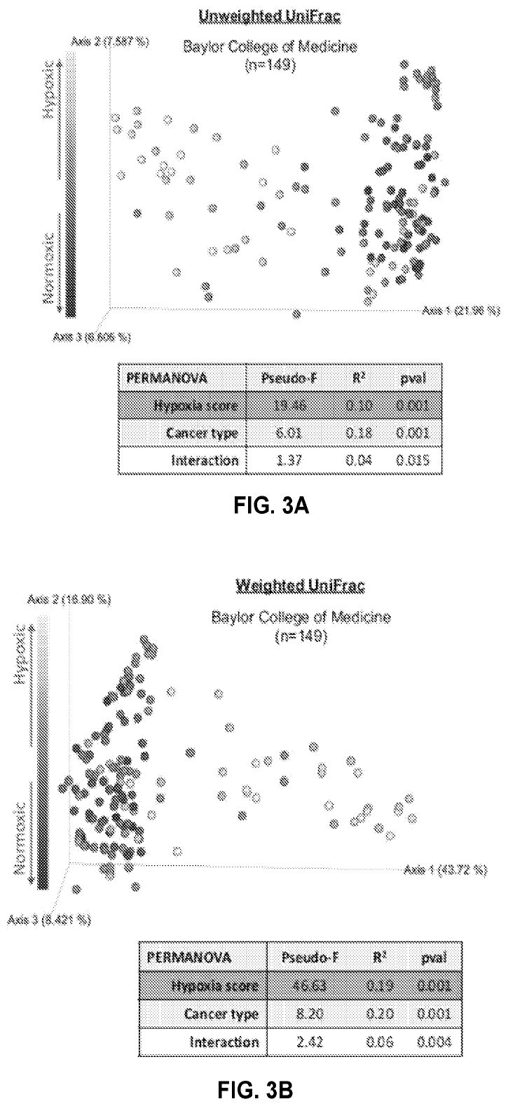

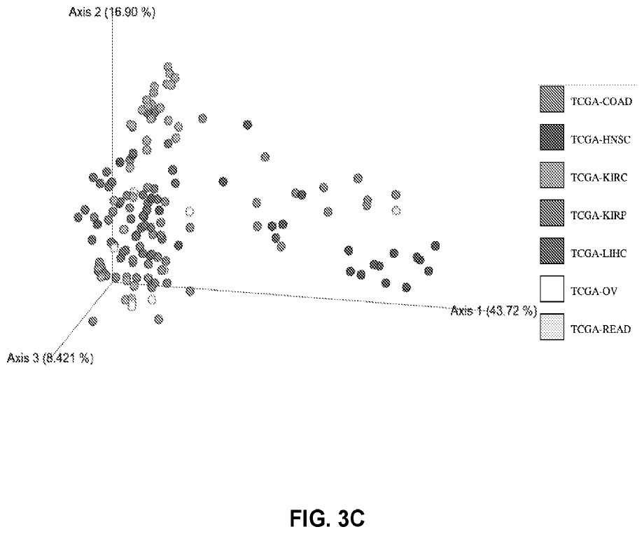

A total of 149 DNA-based primary tumor, treatment-naïve samples, from Baylor College of Medicine processed on Illumina HiSeq machines with matched RNA information (to calculate hypoxia scores) were extracted from TCGA for analysis. The dataset was composed of the following cancer subtypes at their respective quantities: 21 colorectal; 24 head and neck; 31 renal clear cell carcinoma; 26 renal papillary cell carcinoma; 39 liver hepatocellular carcinoma; 6 ovarian serous cystadenocarcinoma; and 2 rectum adenocarcinoma. Analogous to Example 2, distance data were determined for each sample using UniFrac, except that both weighted and unweighted versions were calculated. The resulting PCoA plots are shown in FIGS. 3A-3B, as colored by hypoxia score, and shown in FIG. 3C when colored by cancer type using weighted UniFrac. Weighted UniFrac accounts both for phylogenetic diversity and the abundance of individual microbes in each sample, whereas unweighted UniFrac only accounts for phylogenetic distances between samples based on the presence/absence of microbes. The resulting PERMANOVA analysis results including pseudo-F statistic, R², and p-value calculations are inset under each plot in both FIGS. 3A-3B, and the table under FIG. 3B also applies to FIG. 3C.

Based on the data shown in FIGS. 3A-3B, one can see that the weighted UniFrac data suggests that inclusion of microbial abundances (“weighted” distances) generated a stronger association between microbial hypoxia scores and cancer type than unweighted UniFrac. Notably, the amount of variation in FIG. 3B accounted for by hypoxia approximates that of cancer type (R² of 0.19 vs. 0.20), and the interaction is not significant, suggesting that hypoxia is a pan-cancer driving force of intratumoral microbial compositions.

### Example 4: Correlating Microbial Richness and Phylogenetic Diversity to Hypoxia Metagene Scores

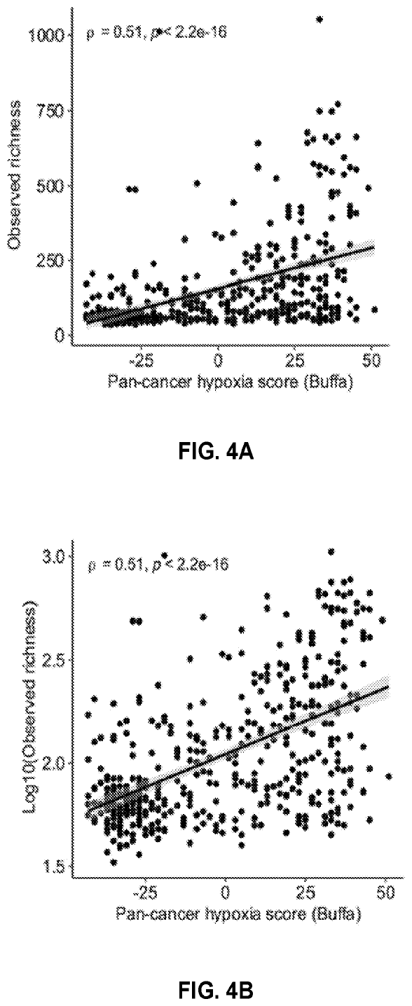

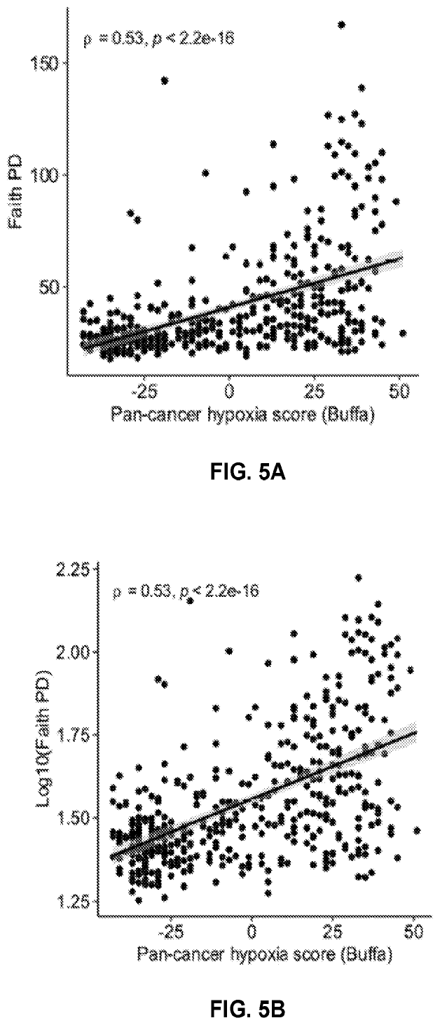

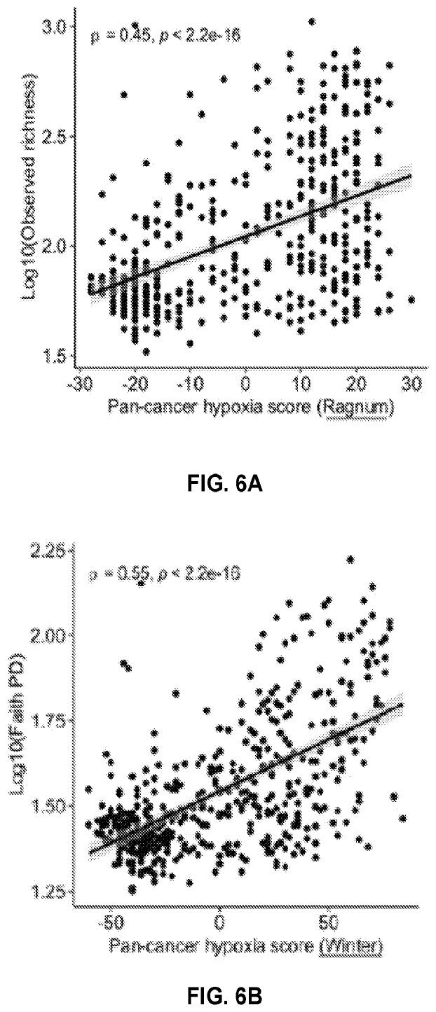

The relationship between Buffa (FIGS. 4A-B, 5A-5B), Ragnum (FIG. 6A), and Winter (FIG. 6B) hypoxic RNA metagene scores were compared to microbial richness (FIGS. 4A-4B, 6A) and phylogenetic diversity (FIGS. 5A-5B, 6B). Microbial richness is the number of unique microbes per sample after rarefaction (i.e., normalizing all samples to the same library size). Faith's phylogenetic diversity (PD) is the sum of the phylogenetic tree branch length that span the members of a set. Phylogenetic diversity can also be understood as a measure of intra-sample diversity based on how phylogenetically divergent microbes within a sample are.

For purposes of this analysis, data as described in Example 2 (453 treatment-naïve tumor sample datasets from Harvard Medical School) were utilized. The three different RNA metagenes-Buffa (Buffa et al. 2010. British Journal of Cancer), Ragnum (Ragnum et al. 2014. British Journal of Cancer), and Winter (Winter et al. 2007. Cancer Research)—comprise different host RNA genes, and the mathematical calculation is computed the same way for each metagene but with varying results depending on the included genes. Specifically, across all samples in the set, the median gene expression value, \tilde{x}, for each gene in the metagene is calculated, and then individual gene expression values in individual samples are compared to the median values. If gene x has a higher expression than the median value for gene x, the sample i is given a score of +1; conversely, if gene x is lower than the median value for gene \tilde{x}, the sample i is given a score of −1. This process is repeated for all genes within the metagene and for all samples within the sample set (cf. Bhandari et al. 2019. Nature Genetics), such that the final hypoxia score is the resultant sum, with an absolute value that is bounded by the number of genes within the metagene. FIGS. 4A-4B show the respective data set samples' observed microbial richness plotted against the Buffa hypoxic RNA metagene scores in both log and non-log domains. Corresponding linear trend lines are also shown with standard error ribbons. The strong linearity shown in FIGS. 4B, 5B, and 6A-6B suggests a log-linear relationship between the degree of intratumoral hypoxia and microbial richness, and that this observation is consistent regardless of the hypoxic metagene used. FIG. 6A similarly shows the respective dataset's log of microbial richness plotted against the Ragnum hypoxic RNA metagene scores. FIGS. 5A-5B show the respective dataset samples' observed microbial phylogenetic diversity plotted against PD and the logarithm thereof, and corresponding linear trend lines are shown. FIG. 6B similarly shows the respective dataset samples' logarithmic base 10 of PD plotted against the Winter hypoxic RNA metagene scores. From FIGS. 4A-4B, 5A-5B, 6A-6B, a log-linear relationship between the various types of metagene hypoxic scores and diversity and/or phylogenetic diversity of samples can be seen. This supports the finding that microbial composition, including presence/absence of microbes, may be useful factors in quantifying tumor hypoxia.

### Example 5: Training and Validating a Classifier Predictive Model with Intratumoral Microbial Abundances and Matched Hypoxia Scores

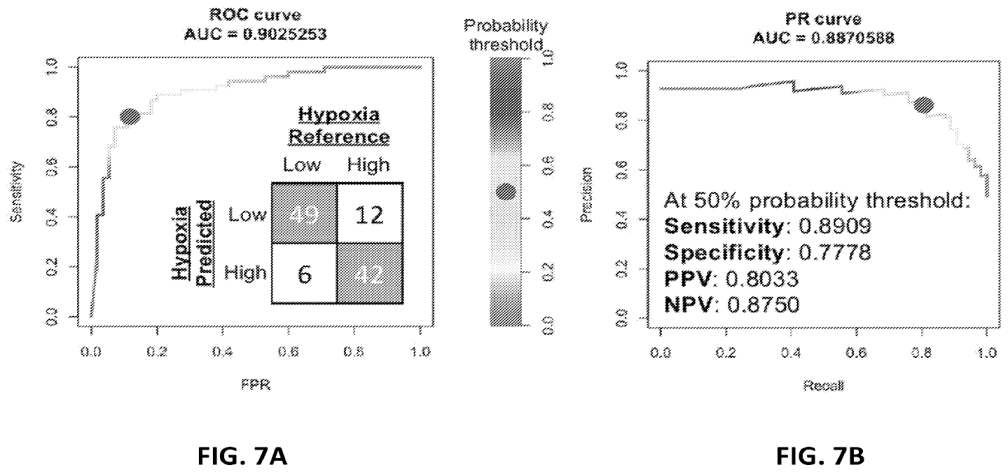

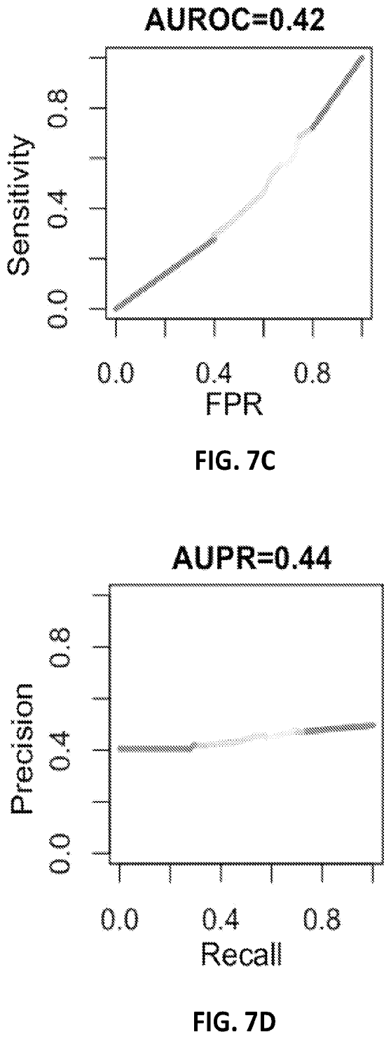

The dataset of intratumoral microbial abundance of previously described in Example 2 (453 treatment-naïve primary tumor samples from Harvard Medical School) and corresponding RNA metagene hypoxia scores were used to train and test a gradient boosting machine predictive model. The sample set was split into training and testing sub-datasets that represented the upper and lower third of the entire range of associated metagene hypoxia scores, such that the upper tertile comprised the most hypoxic samples (labeled “High”), and the lower tertile comprised the least hypoxic samples (labeled “Low”). The middle third of data was discarded. Using these splits, 70% of samples from both the lowest and highest tertiles of hypoxia scores were used to train the classifier and the remaining 30% of both the lowest and highest tertiles were collectively used as an independent, holdout test set. To be clear, the gradient boosting model was trained solely using microbial abundances with matching “High” or “Low” labels for the samples. Receiver operating characteristic (ROC) and precision-recall (PR) curves, shown in FIGS. 7A-7B, were then generated using the trained model's predictions on the 30% holdout test set. Area under the curve (AUC) results for both the ROC (0.902523) and PR (0.8870588) curves are also shown in FIGS. 7A-7B, suggesting a strong ability of microbial abundances to discriminate hypoxic and non-hypoxic (i.e., normoxic) tumors.

To validate the observed association between microbial abundance and hypoxia, as described above, the “Low” and “High” data labels were permuted prior to model training. As can be seen in FIGS. 7C-7D, the model trained on microbial abundances with permuted “Low” and “High” data labels yield AUCs on the 30% holdout test set for the ROC and PR curves equivalent with random guessing (FIGS. 7A-7B). Accordingly, the predictive model generated and described above (FIGS. 7A-7B), is valid and legitimate, and the high-dimensional microbial data does not enable the model to fit a randomly permuted phenotype.

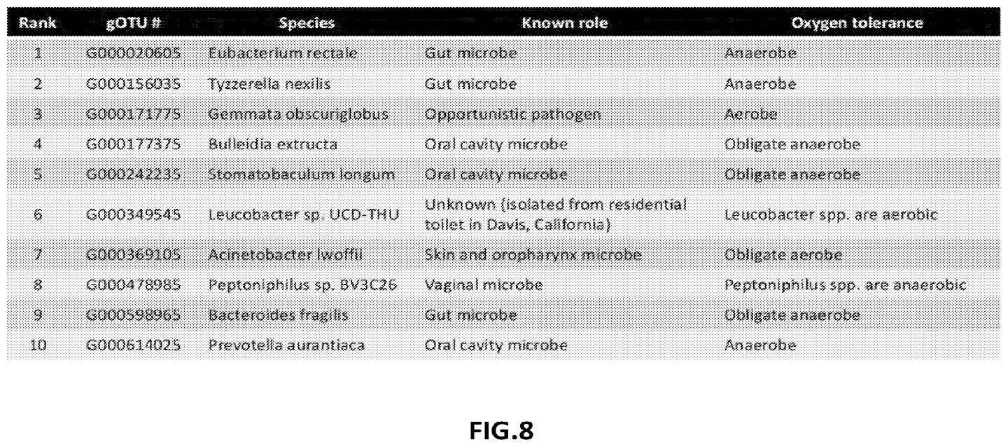

After training the non-permuted predictive model (using 70% of the data, as described above), the trained model was inspected to determine which microbial features were found to be most influential in the model's discrimination between “High” and “Low” hypoxia scores. FIG. 8 shows the rank, description of microbial species, role of microbe (if known), known oxygen tolerance, and microbial genome ID number (“gOTU #”). Of note, the genome ID number represents the microbial genome to which DNA sequencing information aligned during the taxonomy identification step.

### Example 6: Training and Validating a Regression Predictive Model with Intratumoral Microbial Abundances and Matched Hypoxia Scores

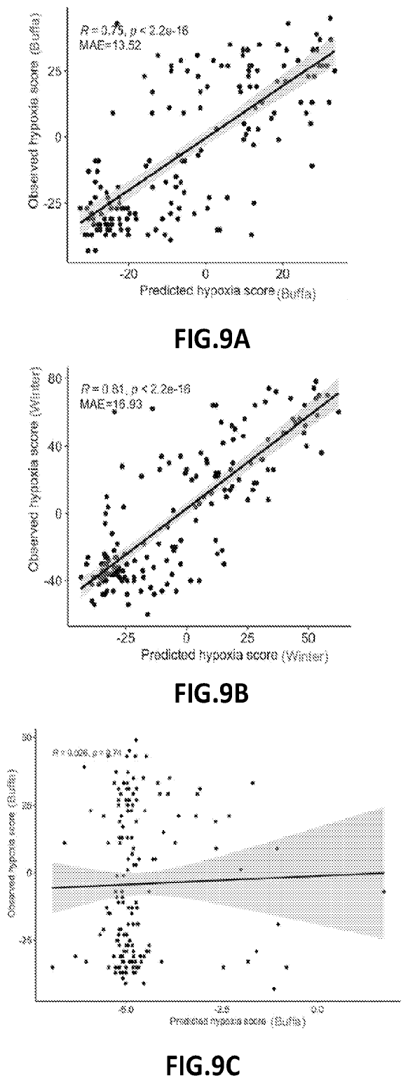

A regression predictive model was trained with the same data as described in Example 2 (453 treatment-naïve primary tumor samples from Harvard Medical School). The same tertile data splitting strategy with 70% training and 30% testing as explained Example 5 was used with the difference that the samples were not binned and labeled based on hypoxia score. Rather, the numerical value of the hypoxia score was used for training and testing of a regression-based gradient boosting predictive model. Also like Example 5, only the microbial abundances of the training set were used to train the predictive model. Resulting plots of observed and predicted hypoxia scores on the 30% holdout test set using the regression predictive model can be seen in FIGS. 9A-9B for Buffa and Winter metagenome observed hypoxia scores, respectively. Inset on the plots are the Pearson correlation coefficients, their associated p-values (based on hypothesis test of slope), and the mean absolute errors (MAE).

In a similar manner as the validation process described in Example 5, the numerical hypoxia scores labels were permuted during training and then the trained model was applied on the 30% holdout test set. This analysis represents a negative control that should yield no association. Accordingly, the Pearson correlation of approximately zero between the actual and predicted hypoxia scores can be seen in FIG. 9C, again validating the legitimacy of the non-permuted model associations between intratumoral microbial abundances and degrees of hypoxia.

### Example 7: KEGG and MetaCyc Predictive Models

Using the same dataset as described by Example 2 (453 treatment-naïve primary tumor samples from Harvard Medical School), two sets of classifier predictive models based on KEGG (Kyoto Encyclopedia of Genes and Genomes), and MetaCyc Pathway and Module abundances were trained and tested using the same tertile data splitting and 70% training-30% testing strategy described in Example 5. Here, a module describes with high specificity a gene set that gives rise to a particular function (e.g., one module may comprise microbial genes involved in the citrate cycle, another module may comprise microbial genes involved in glycolysis). Pathways, on the other hand, comprise abstraction of modules and may combine multiple modules (e.g., “metabolic pathways” or “microbial metabolism in diverse environments” are KEGG pathways that would encompass both citrate cycle and glycolysis). In order to convert from microbial abundances to functional module or pathway abundances, the nucleic acid molecule alignments against microbial genomes were translated to amino acids, followed by mapping against known proteins in the UniProt database, followed by mapping those proteins against lists of proteins known to be involved in modules or pathways. The abundances of the modules or pathways are thus reflective of how represented their coding sequences are in the biological sample.

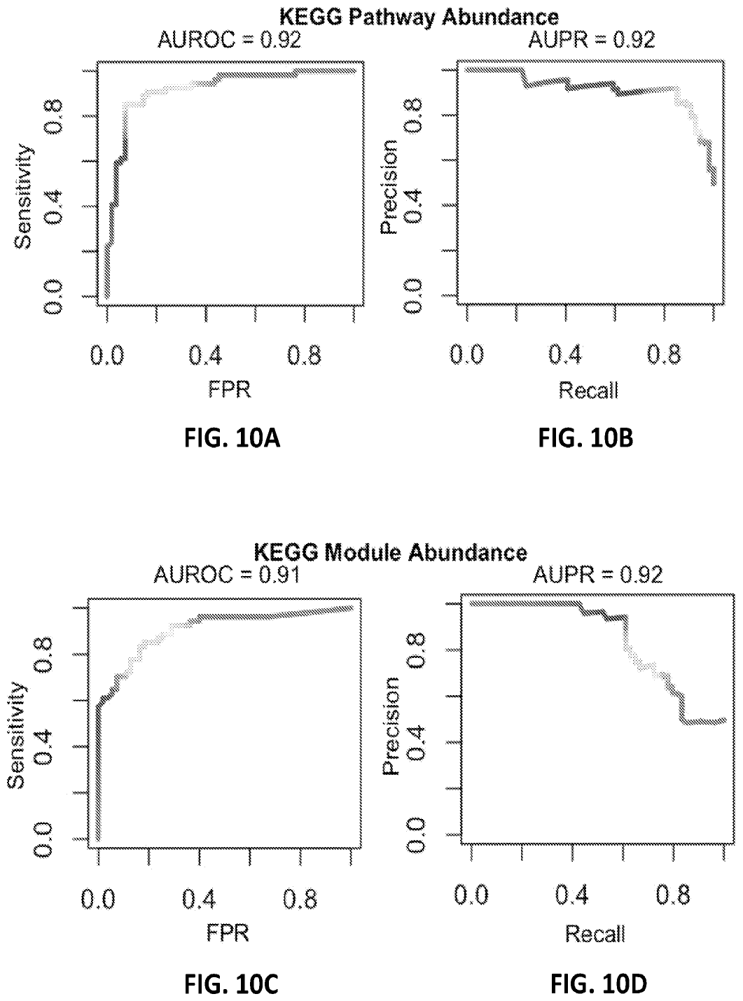

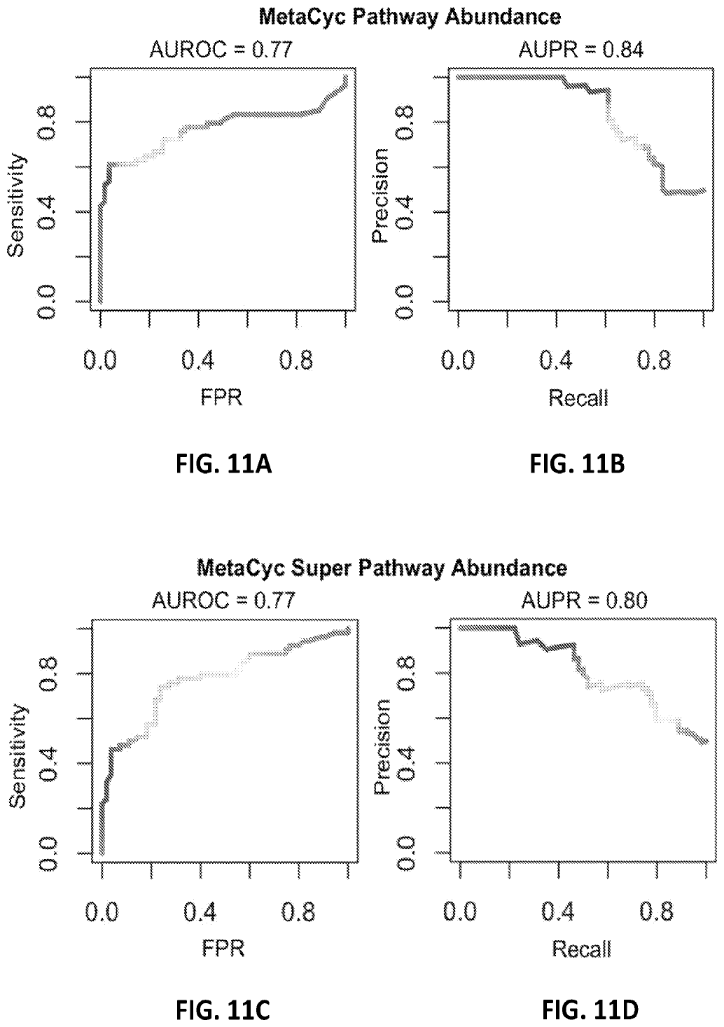

After training the gradient boosting models on the module or pathway abundances from 70% of the samples (training set), the trained model was applied on the remaining 30% holdout test set to generate predictions and compare with the ground truth. The resulting ROC and PR curves with corresponding AUC for each trained predictive model was determined on the 30% holdout test sets: KEGG pathway (FIGS. 10A-10B), KEGG module (FIGS. 10C-10D), MetaCyc pathway (FIGS. 11A-11B), and MetaCyc super pathway (FIGS. 11C-11D).

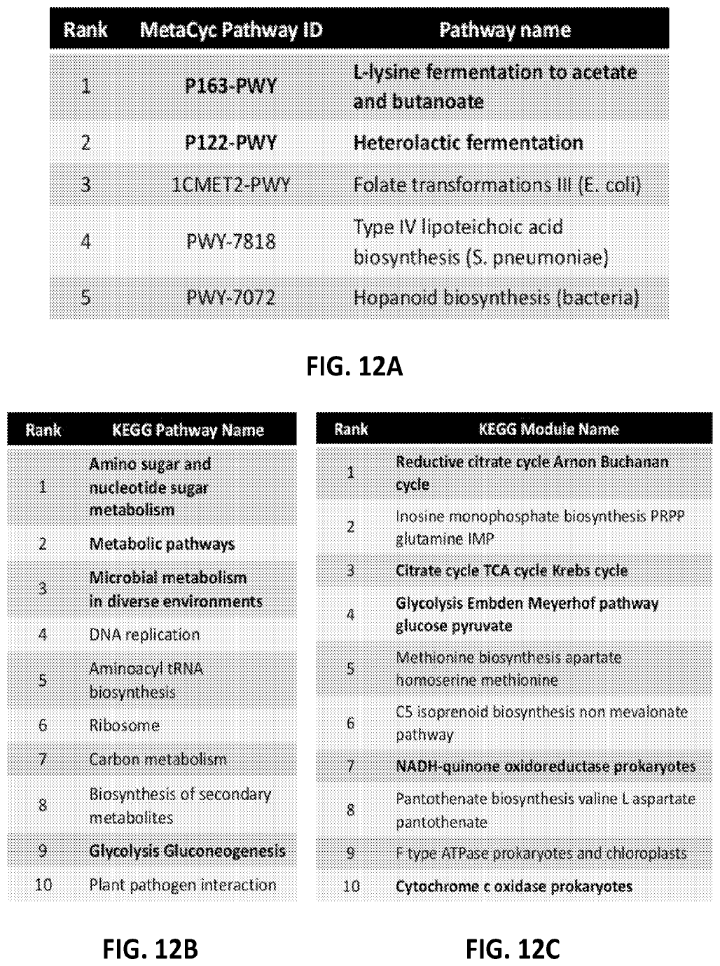

Similar to Example 5, upon completing training of the KEGG and MetaCyc pathway and module models, the resulting top 10 ranked features for KEGG pathway (FIG. 12B), KEGG module (FIG. 12C), and MetaCyc pathway (FIG. 12A) useful for determining “High” or “Low” sample hypoxia were identified.

### Example 8: Diversity of Bacterial Oxygen Tolerances in Major Human Cancer Types

A bacterial phenotyping pipeline was developed to assess oxygen tolerances of bacteria found in eight (8) major human cancer types: breast, lung, melanoma, pancreas, ovary, bone, glioblastoma, colon. Briefly, a dataset of intratumoral bacterial abundances comprising 1010 tumor samples across nine (9) medical centers was used purposes of this Example (sourced from Nejman et al. 2020. Science) since it was subjected to a series of successive decontamination steps that performed the following: remove general common contaminants (filter 1), remove contaminants detected by DNA extraction batch (filter 2), remove contaminants detected by PCR batch (filter 3), remove contaminants detected by sequencing lane batch (filter 4), remove contaminants detected in non-biological paraffin samples (filter 5), remove contaminants not found in multiple medical centers (filter 6). The implementation of these decontamination filters provided confidence that subsequent determination of bacterial oxygen tolerance distributions within these cancer types would not be affected by contaminants.

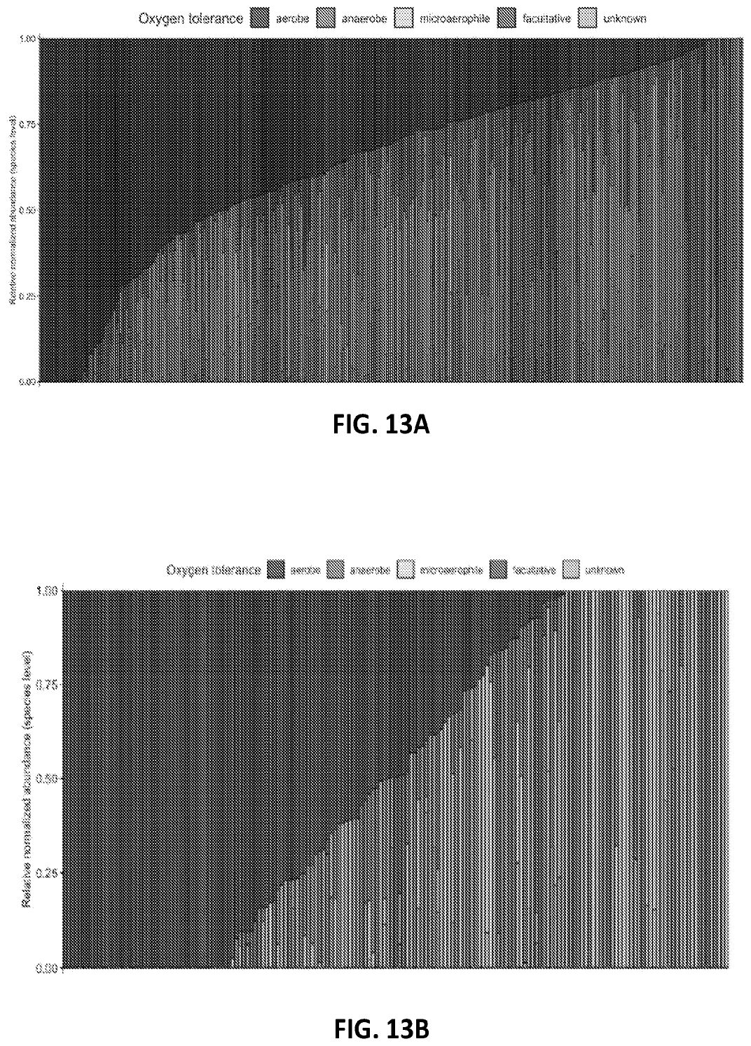

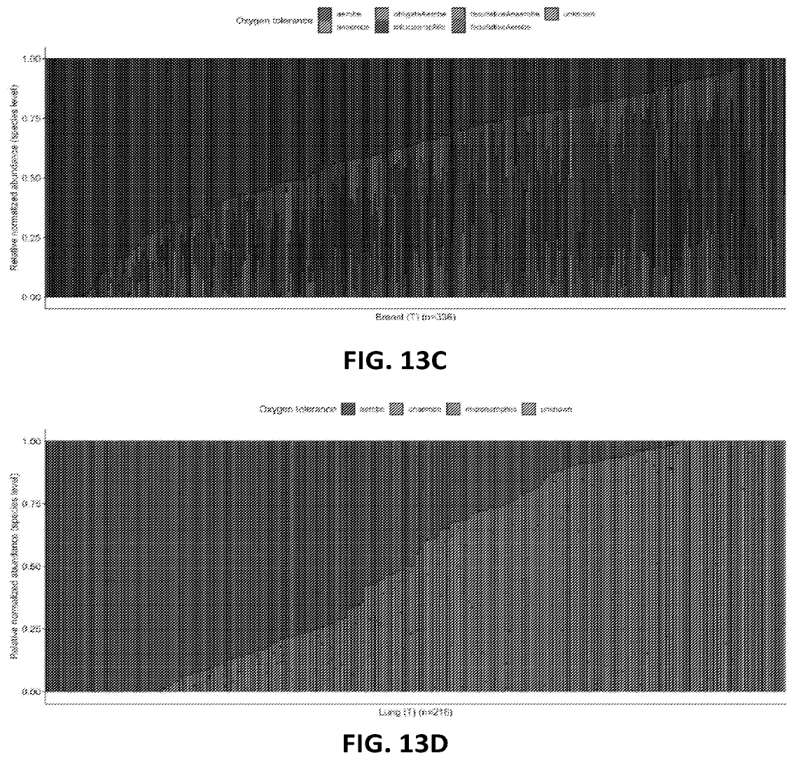

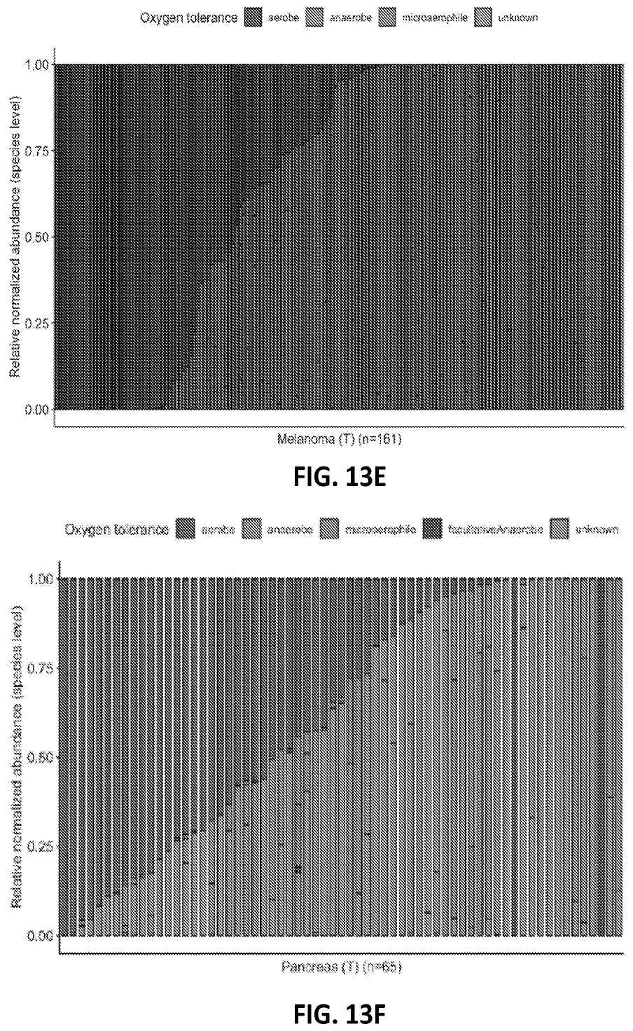

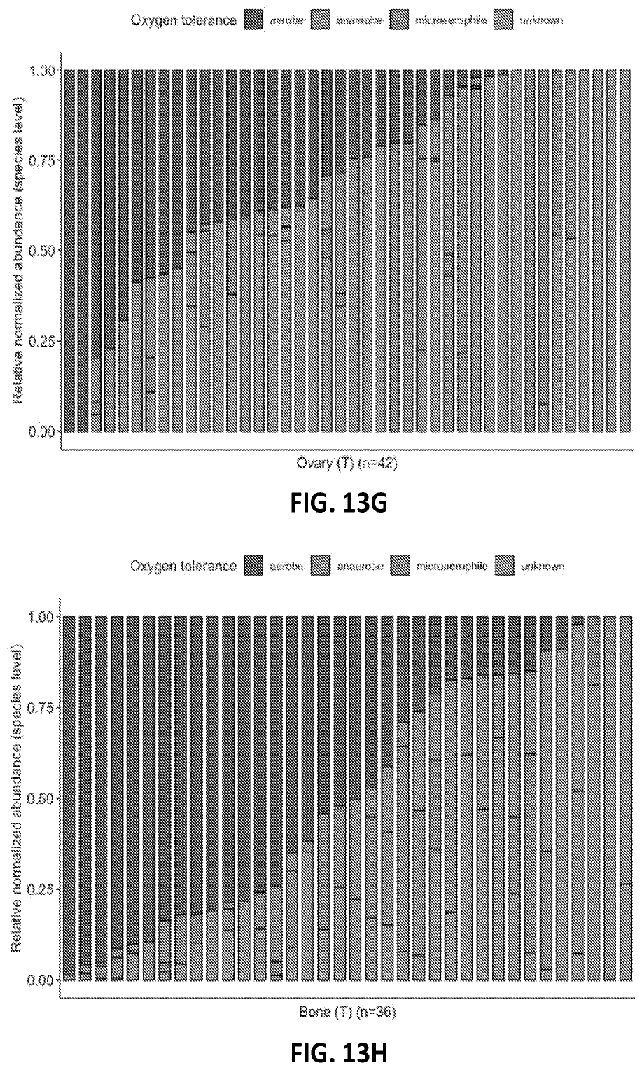

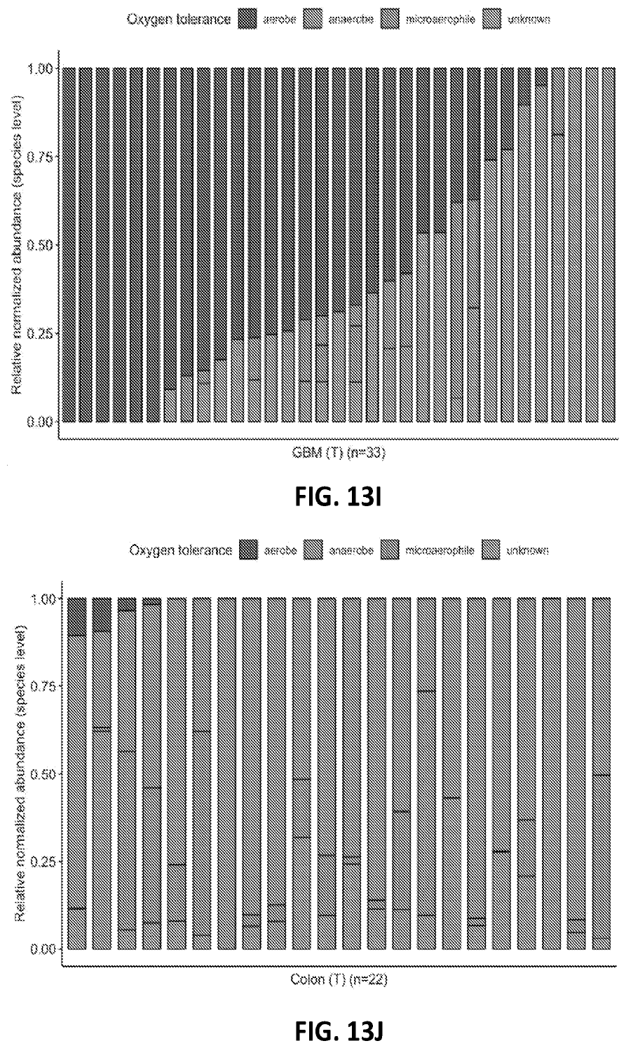

With the decontaminated data, all known bacterial oxygen tolerances and their relative abundances were calculated for each tumor sample across each cancer type. Specifically, using a bacterial isolate phenotype database, BacDive (Reimer et al. 2019. Nucleic Acids Research), oxygen tolerances for all catalogued bacterial isolates of all decontaminated bacterial species in the cancer dataset of a given sample were determined. For cases in which a multiple bacterial isolates or associated oxygen tolerances existed, a majority vote approach was used to label the final oxygen tolerance of a particular decontaminated species; moreover, in cases where no matching bacterial isolates existed in BacDive, or when oxygen tolerances were conflicting in a manner not resolvable using majority voting (i.e., equal representation of multiple, differing isolate oxygen tolerances), the particular decontaminated bacteria's oxygen tolerance was labeled “unknown.” The following nine (9) oxygen tolerance label types were available to be assigned through BacDive as a bacterial phenotype: obligate aerobe, aerobe, facultative aerobe, microaerophile, aerotolerant, microaerotolerant, facultative anaerobe, anaerobe, obligate anaerobe. Once the oxygen tolerances for all decontaminated bacteria in the dataset had been identified using the aforementioned process, their relative abundances were calculated within samples. To be clear, the relative abundances for all decontaminated bacteria were calculated, including those with “unknown” oxygen tolerances. These relative abundances, which sum to one (1), were then plotted in stacked bar graph format and colored by oxygen tolerances. As a hypothetical example, if sample y contained bacteria A, B, C comprising an aerobe, aerobe, and anaerobe, respectively, then the stacked bar plot would have two colors, one for aerobes, summing the relative abundances of bacteria A and B, and one for anaerobes, comprising bacterium C. The results of such analysis for all decontaminated bacteria across all intratumoral samples from eight (8) cancer types is shown in FIGS. 13A-13J. In each of the plots of FIGS. 13A-13J, the y-axis represents the proportion of decontaminated bacterial species classified to have a particular oxygen tolerance from the BacDive-based phenotyping pipeline (described above), whereas the x-axis of FIGS. 13A-13J represent individual samples (represented by a vertical bar) of a collection of individual cancer types.

Initially, all decontaminated bacteria that passed all six filters (described above) were processed using the phenotyping and relative abundance analyses for breast cancer (FIG. 13A) and lung cancer (FIG. 13B). Since the sixth filter is known to be extremely conservative (cf. Nejman et al. 2020. Science) and may too stringently restrict the number of detected bacteria, the samples were reprocessed using bacterial species that passed through the first five of the six filters (FIGS. 13C-13J) across all eight (8) cancer types.

From FIGS. 13A-13J, it can be seen that: (1) not all or even most bacteria in tumors are anerobic but rather many aerobes, microaerophiles, and so forth exist across different cancer types; (2) the simultaneous distribution of aerobes, microaerophiles, and anaerobes (among others) in single samples of decontaminated data suggests that oxygen gradient(s) must exist within tumor that enable their co-existence, since each of them tolerate different oxygen concentrations; and (3) the particular distribution of bacterial oxygen tolerances may indicate the degree of hypoxia, as observed, for example, by the predominant number of anaerobes in colorectal cancer specimens (FIG. 13J) with the understanding that the colorectum is an anaerobic environment with a high degree of hypoxia (cf. Singal & Shah. 2020. JBC).

### Example 9: Determining Tumor Oxygenation from a Tissue Tumor Biopsy

The methods and systems of the disclosure provided herein can be used to determine tumor oxygenation characteristic from a tumor biopsy. The steps involved in this method may include: (1) obtaining a tissue segment of tumor from a subject; (2) extracting the DNA from the tissue, for example with the ZymoBIOMICS DNA Miniprep Kit; (3) preparing DNA sequencing libraries from the extracted DNA, such as using the KAPA HyperPlus Kit; (4) sequencing the DNA libraries with next generation sequencing (NGS), such as on an Illumina NovaSeq 6000 instrument; (5) aligning the DNA sequencing reads output against a database of microbial genomes, contigs, bins, and/or metagenome-assembled genomes (MAGs) to obtain a table of microbial abundances for the sample (e.g., using the SHOGUN algorithm by Hillmann et al. 2020. Bioinformatics); and (6) inputting the table of microbial abundances into a trained machine learning algorithm to obtain a prediction of the patient's tissue oxygenation characteristic. The same machine learning model or another model may use the inferred tumor oxygenation characteristic to prognose patient survival, tumor aggressiveness, and/or therapy response. An example of the machine learning algorithm would be gradient boosting classification trees or regression. In another case, microbial DNA may be enriched in the sample prior to sequencing (e.g., amplification of the 16S rRNA subunit). In another case, the host DNA may be depleted prior to sequencing (e.g., Marotz et al. 2018. Microbiome; Charalampous et al. 2019. Nature Biotechnology). In yet another case, the sequencing may dynamically reject non-microbial DNA while sequencing microbial DNA as a form of microbial enrichment (e.g., Payne et al. 2020. Nature Biotechnology). In still another case, multiple forms of host depletion and microbial enrichment may be joined to further enrich the microbial nucleic acid molecule signal. To someone skilled in the art, it is evident that these steps can also be used with a focus on microbial RNA, epigenetic marks, or proteins. Other alternatives include combinations of microbial DNA and RNA with host DNA, RNA, epigenetic marks, and/or proteins that may increase the accuracy of diagnosing tumor oxygenation characteristic. An optional step of the method presented above comprises decontaminating the table of microbial abundances, removing contaminants, and retaining non-contaminants. The decontamination process may improve the accuracy and/or prediction of the machine learning algorithm.

### Example 10: Determining Tumor Oxygenation from a Liquid Tumor Biopsy

The methods and systems of the disclosure provided herein can be used to determine a patient and/or subject's tumor oxygen characteristic from a liquid biopsy. The method may include the following steps: (1) obtaining a liquid biopsy from a subject with cancer; (2) extracting the DNA from the liquid biopsy, for example with the ZymoBIOMICS DNA Miniprep Kit; (3) preparing DNA sequencing libraries from the extracted DNA, such as using the KAPA HyperPlus Kit; (4) sequencing the DNA libraries using next generation sequencing (NGS), such as on an Illumina NovaSeq 6000 instrument; (5) aligning the outputted DNA sequencing reads against a database of microbial genomes, contigs, bins, and/or metagenome-assembled genomes (MAGs) to obtain a table of microbial abundances for the sample, such as using the SHOGUN algorithm (Hillmann et al. 2020. Bioinformatics); and (6) inputting the table of microbial abundances into a trained machine learning algorithm in order to obtain a prediction of the tumor's oxygen characteristic with a certain certainty. The same machine learning model or another model may use the inferred tumor oxygen characteristic(s) to prognose patient survival, tumor aggressiveness, and/or therapy response. An example of the machine learning algorithm is a gradient boosting classification trees or regression. In another case, microbial DNA may be enriched in the sample prior to sequencing (e.g., amplification of the 16S rRNA subunit). In another case, the host DNA may be depleted prior to sequencing (e.g., Marotz et al. 2018. Microbiome; Charalampous et al. 2019. Nature Biotechnology). In yet another case, the sequencing may dynamically reject non-microbial DNA while sequencing microbial DNA as a form of microbial enrichment (e.g., Payne et al. 2020. Nature Biotechnology). In still another case, multiple forms of host depletion and microbial enrichment may be joined to further enrich the microbial nucleic acid molecule signal. To someone skilled in the art, it is evident that these steps can also be used with a focus on microbial RNA, epigenetic marks, or proteins. Other alternatives will include combinations of microbial DNA and RNA with host DNA, RNA, epigenetic marks, and/or proteins that may provide a more accurate diagnosis of the tumor hypoxic status. In addition to the method provided above, the method may include a step of decontaminating the table of microbial abundances, removing contaminants, and retaining non-contaminants to improve the accuracy of the trained machine learning model in predicting the subjects' tumor oxygenation characteristic.

### Example 11: Bacterial Theranostics

The methods and systems of the disclosure provided herein may be used to determine one or more microbes that can be used as a bacterial thernanostic. The steps of such a method may include: (1) using a database of intratumoral microbes with varying oxygen tolerances, selecting one or more microbes whose thriving or metabolic activity changes based on oxygen concentrations and/or whose thriving affects local host angiogenesis; (2) engineering these one or more microbes with one or more reporter genes that will be measured using known imaging techniques and/or a reporter gene that produces a metabolite secreted into circulation; and (3) administering the bacteria to the patient, such as in the form of a probiotic via the gastrointestinal tract, or directly as an injection near to or in the direction of the patient's tumor (e.g., intravenous, intramuscular, and/or intratumoral injections). In one case, the one or more engineered microbes are used to diagnose tumor growth and/or increasing tumor hypoxia. In another case, the one or more engineered microbes are used to treat tumors that have increasing tumor hypoxia. In yet another case, detection of the one or more reporter genes or metabolites is used to prognose a subject, such as their estimated survival or their predicted response to therapy.

### Example 12: Spatial Assaying of Oxygen Sensitive Microbial Populations

The methods and systems of the disclosure provided herein will be used to spatially assay oxygen sensitive microbial populations in the tissue of a subject. The method will include the following steps: (1) using a database of intratumoral microbes with varying oxygen tolerances and/or cancer prevalence, selecting one or microbes to target; (2) developing oligonucleotides that uniquely target one or more of the targeted microbes of varying oxygen tolerances, such as those shown in Table 1; (3) obtaining a tissue sample from a subject and applying those oligonucleotides to the tissue sample in order to obtain a spatial readout of the locations of one or more targeted microbes with varying oxygen tolerances; and (4) using the spatial readout of the locations of one or more targeted microbes with varying oxygen tolerances to determine the tissue's oxygen characteristic. In certain cases, all microbes within the tissue sample will be assayed without the development or utilization of targeted oligonucleotides. In certain cases, oligonucleotides will be developed and utilized that are specific to non-microbial targets in tandem with oligonucleotides that are specific to microbial targets. In other cases, the oligonucleotides will hybridize against microbial DNA, microbial RNA, non-microbial DNA, and/or non-microbial RNA, or any combination thereof. In still other cases, the oligonucleotides will be conjugated to antibodies in order to spatially assay microbial and/or non-microbial protein targets, or any combination thereof. In certain cases, the subject's tissue sample comprises a tumor sample. In certain cases, the spatial readout of the one or more targeted microbes will be obtained via imaging. In certain cases, the spatial readout of the one or more targeted microbes will be obtained via sequencing the microbial-specific oligonucleotides and/or spatially encoded oligonucleotides. In still other cases, the spatial readout of the one or more targeted microbes will be used to inform the prognosis of the subject, such as subject survival or predicted response to a therapy. In other cases, the spatial readout of the one or more targeted microbes will inform the selection of a particular therapy for the subject.

### Example 13: Tissue Microbial Diversity and Hypoxia Gradients in Human Cancerous Tissue

Hypoxia gradients of cancer tissue samples excised and embedded in paraffin were analyzed using spatial RNA transcriptomics to determine a spatial distribution of human and microbial nucleic acid molecule targets. Representative histologic sections of fifteen paraffin embedded human cancer tissues and one germ-free mouse tissue, namely: 6 breast, 7 lung, and 2 melanoma cancerous tissues, and one germ-free mouse non-cancer tissue, were analyzed across 336 areas of interest across all sixteen samples.

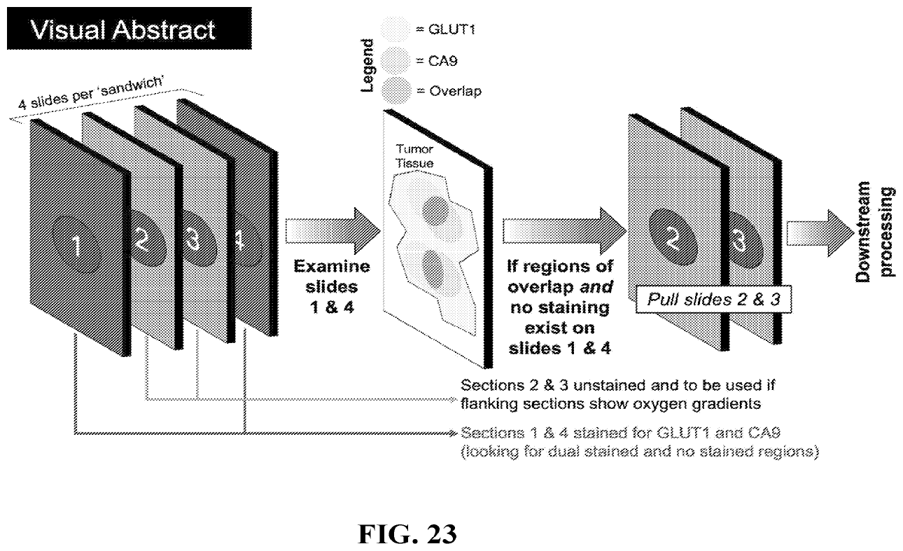

Representative histological sections for each cancerous tissue sample were selected for analysis based on the presence of hypoxic gradients across the histological section. A four-slide sandwich approach, as shown in FIG. 23, was utilized to determine representative histological sections of a given cancerous tissue for further downstream processing. Initially, slides 1 and 4 of the four-slide sandwich were screened for the presence hypoxic gradients by immunohistochemistry staining with GLUT1 and CA9 hypoxic markers. If the first and fourth slides both expressed hypoxic and normoxic regions, i.e., they expressed GLUT1, CA9, a region of overlap of the two markers, and regions that do not express the GLUT1 and CA9 markers, a determination was made that slides 2 and 3 sandwiched by slides 1 and 4 are representative histological sections of a hypoxia gradient for the given cancerous tissue sample. Slides 2 and 3 were then subjected to further downstream human and/or microbial RNA spatial transcriptomics and/or RNA or protein staining analysis.

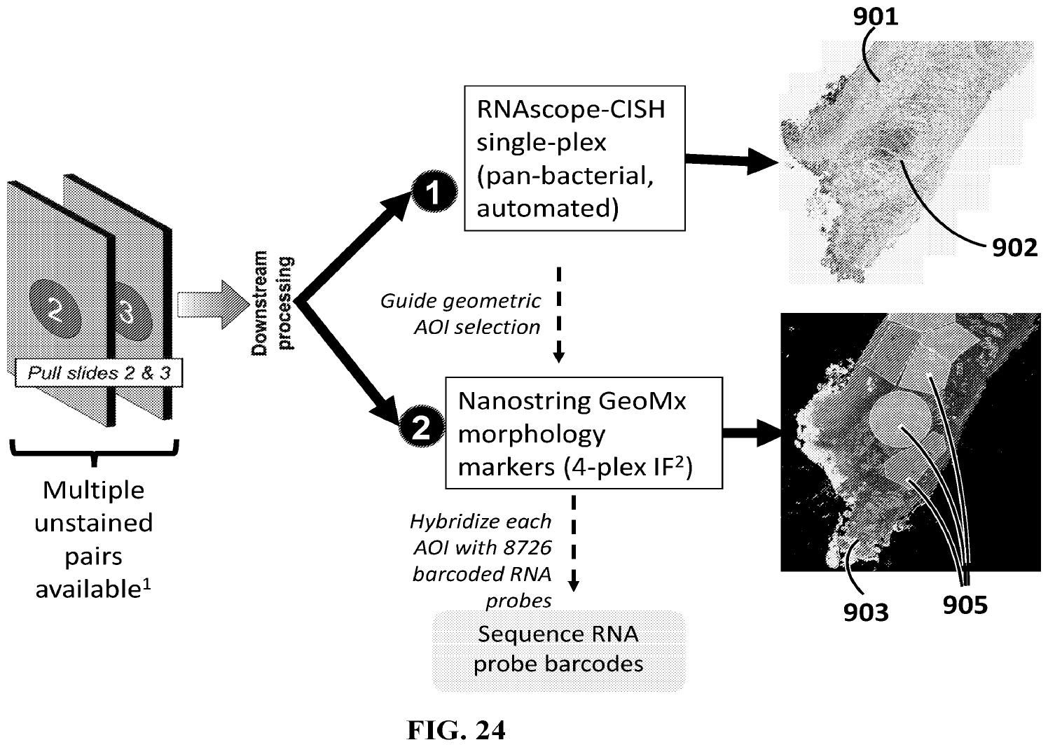

Next, regions of interest (ROIs) of the representative histologic section for each cancer were analyzed to determine one or more ROIs to probe with spatial RNA transcriptomic analysis (e.g., using Nanostring GeoMx spatial transcriptomics platform), as shown in FIG. 24. One of the two unstained representative histologic sections (e.g., slides 2 and 3 above), determined from the above four slide sandwich, was stained using RNAscope chromogenic in-situ hybridization (CISH) with a 16S rRNA pan-bacterial marker. An example image 901, of a representative histologic section stained with RNAscope CISH for 16S rRNA is shown in FIG. 24. The RNAscope CISH stain for 16S rRNA (pan-bacteria) nucleic acid molecules shows a brown yellowish color 902, as seen in FIG. 24. Regions of bacterial 16S rRNA nucleic acid molecules identified by the RNAscope CISH and regions of hypoxia (e.g., GLUT1 staining) were then used to inform the selection of one or more corresponding areas of interest (AOIs) 905 of the representative histology section (slide 3) 903 to probe with the GeoMx's human and microbial RNA transcriptomic analysis.

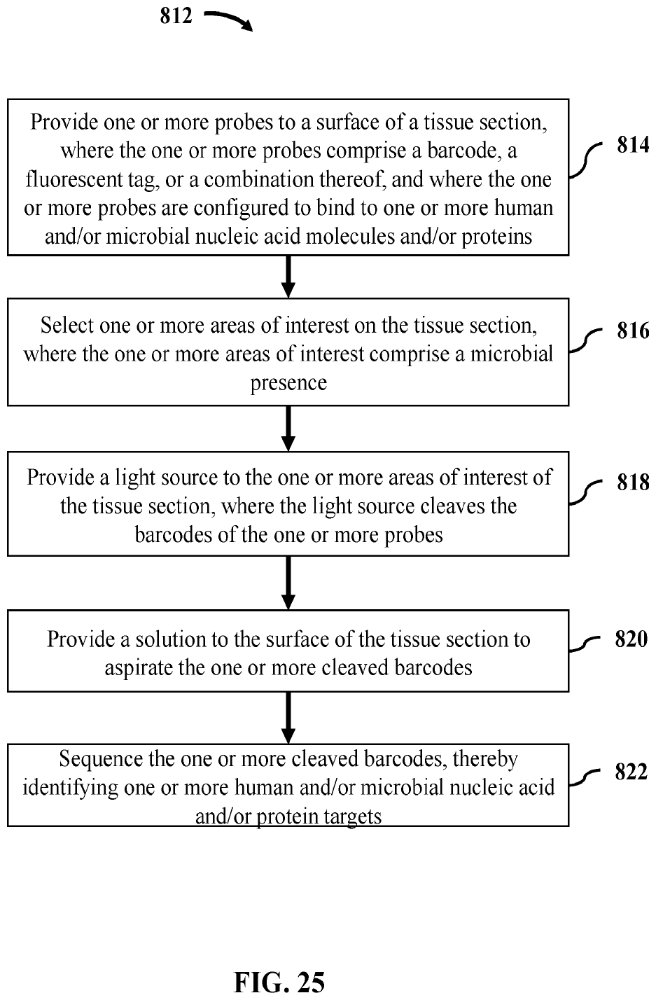

Each identified AOI for all the representative histological sections of all the cancerous tissue was interrogated with 8,726 UV-cleavable barcoded RNA probes (814) that were configured to bind to human and microbial nucleic acid molecule targets on the representative histologic section. As shown and described by the method 812 (FIG. 25), using the prior identified AOIs for each representative histologic section (816), the GeoMx spatial transcriptomics system selectively UV cleaved barcodes (818) of the corresponding RNA probes (814) that were bound to a particular AOI. The resulting UV-cleaved barcodes were then aspirated and sequenced (820, 822).

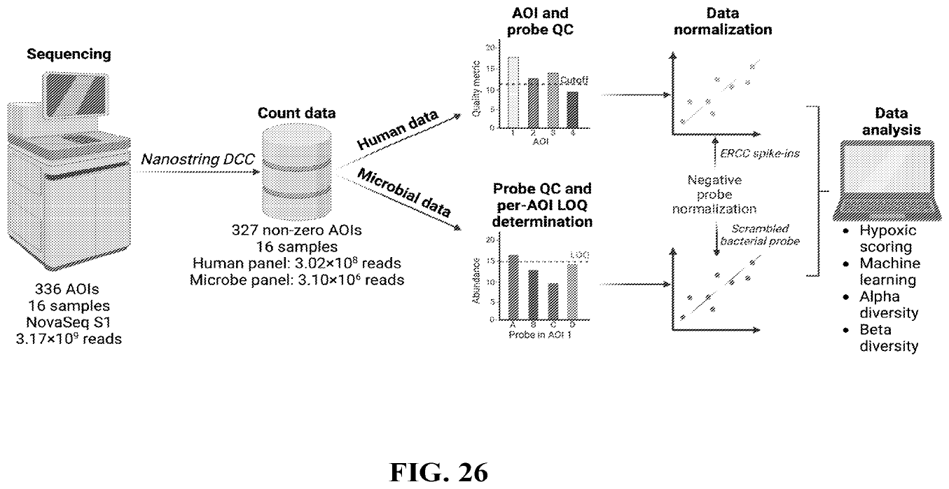

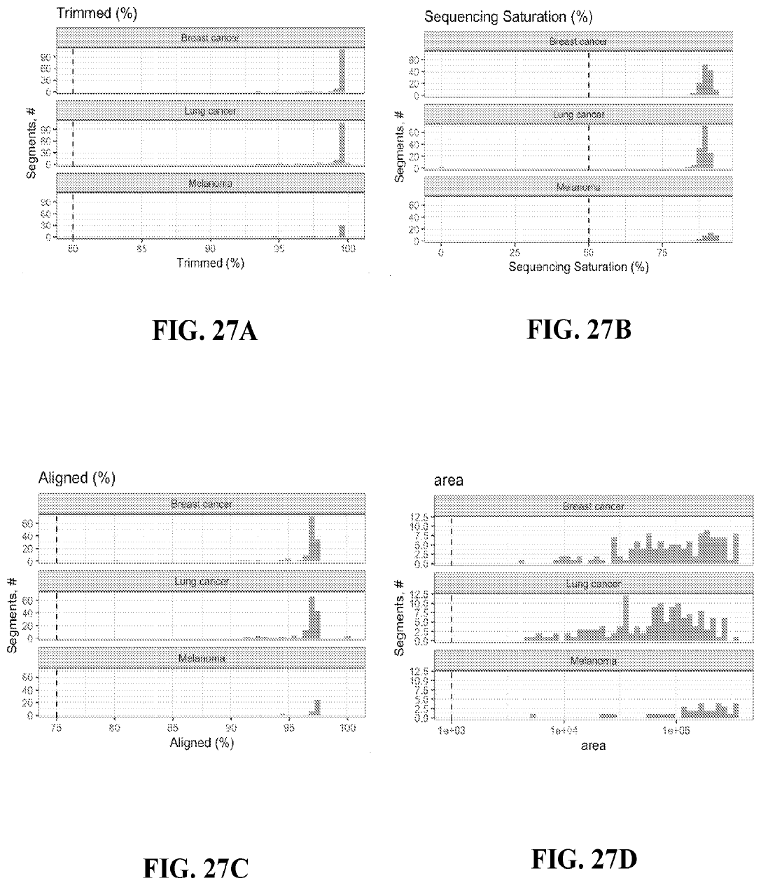

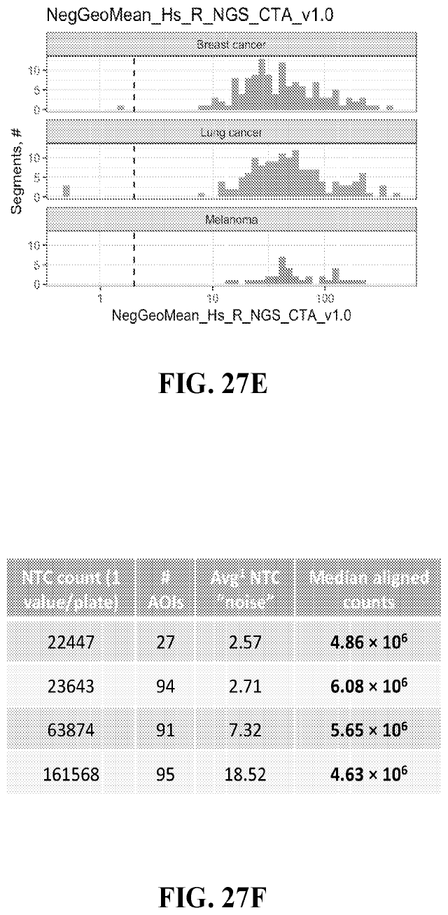

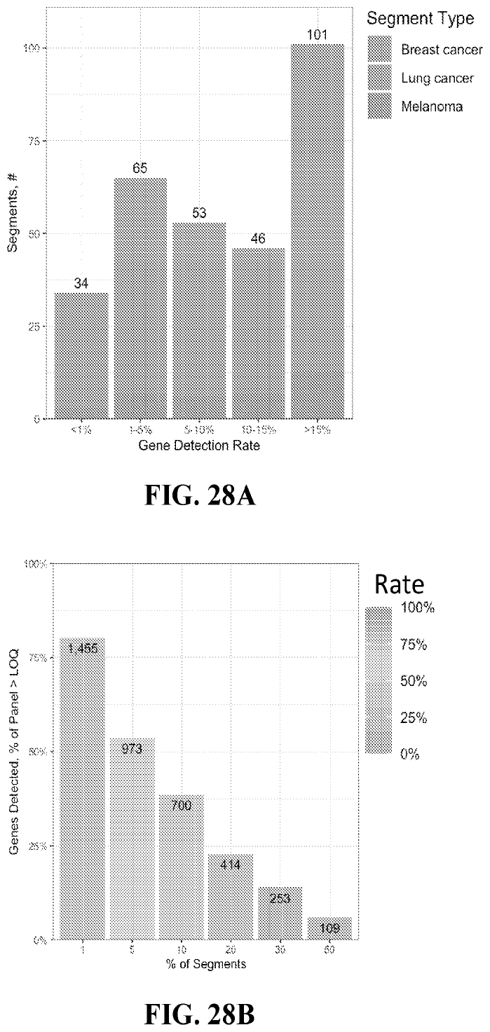

The resulting barcodes across ˜300 AOIs of 16 samples that were deeply sequenced (3.17×10⁹) reads, as shown in the sequencing workflow diagram of FIG. 26. The human sequencing data and the microbial sequencing data was then split for independent normalization. The human sequencing reads were normalized against ERCC spike-in RNA probes that were included in each AOI for each tissue sample processed by GeoMx spatial transcriptomics. The ERCC spike-in RNA probes did not have any known complementary sequence configured to bind to human nucleic acid molecules and thus act as a measure of background of noise of probes binding to paraffin (e.g., paraffin stickiness) for each AOI. In a similar manner, a scrambled pan bacterial probe was used to approximate the noise floor and/or threshold in normalizing the microbial sequencing reads. The normalized human sequencing reads were then used to determine hypoxic scores that were compared against corresponding microbial compositions, alpha diversity, and beta diversity of the AOIs within and between cancer tissue samples. Quality control factors, as shown in FIGS. 27A-27E all were above the dotted line quality threshold across all cancerous tissue types and all quality check factors. Non-template counts for both human and microbial data was measured and averaged against the 8,726 UV-cleavable barcodes, as shown in FIG. 27F. In view of the median align counts of around 4-6×10⁶ provided by the deep sequencing, average NTC noise of 2-20 was identified to be negligible.

Quality control (QC) was also performed on both a local probe QC and a global probe QC levels. Local probe QC removed a probe if the probe is an outlier according to Grubb's test for outliers for a particular probe in a given AOI. Global Probe QC removed a probe if: (1) its geometric mean divided by the geometric mean of all probe counts against the gene target from all AOIs was less than 0.1, and (2) if the probe is an outlier by the Grubb's test for outliers in greater than 20% of the AOIs. After local and global probe QC, a total of 8642 probes, corresponding to 1812 genes across 299 AOIs remained.

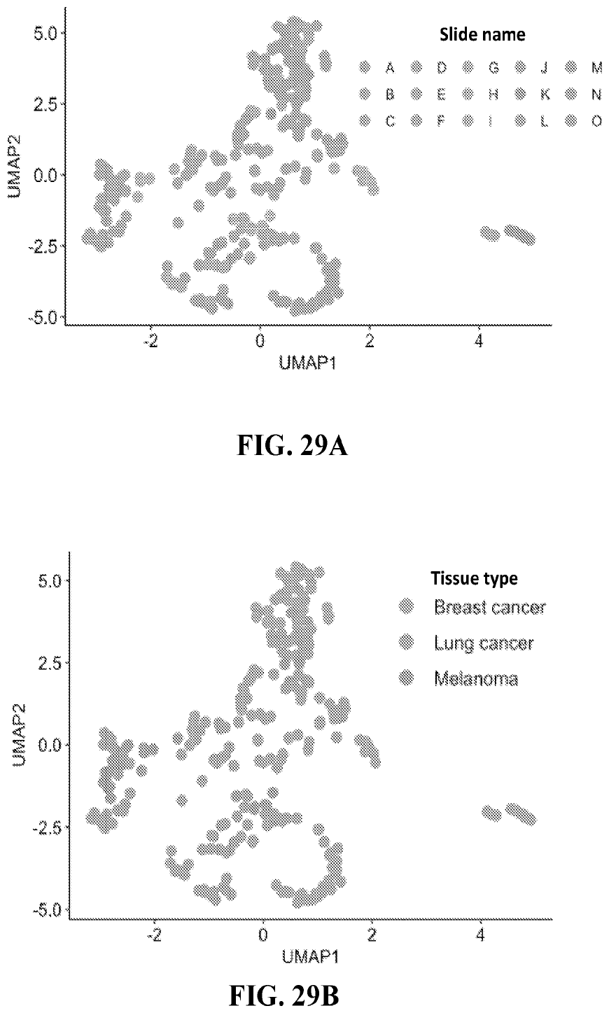

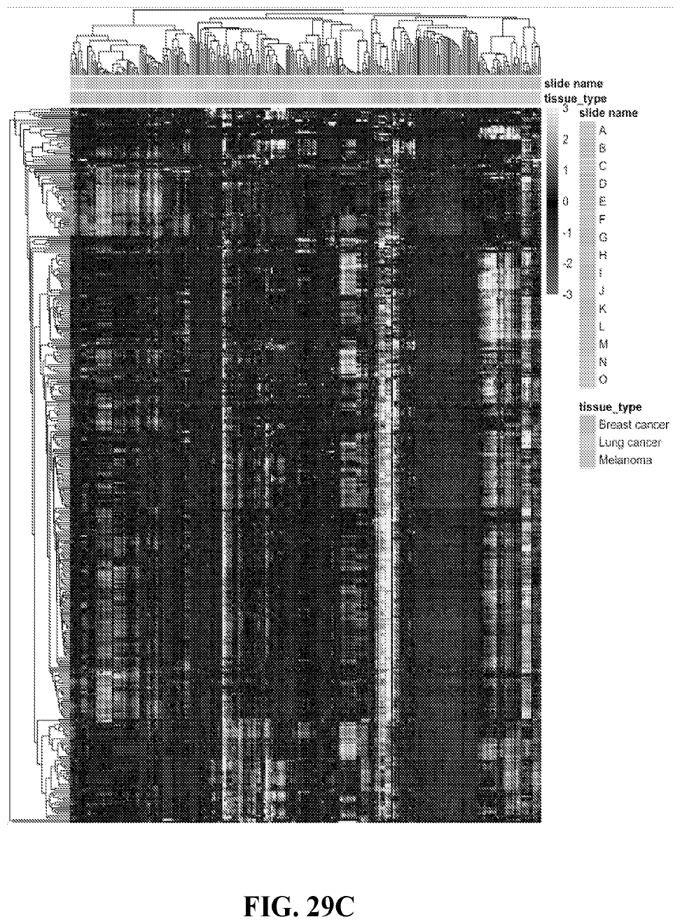

Following normalization and probe QC, the human transcriptomic data was subjected to uniform manifold approximation and projection (UMAP) and clustering, as shown in FIGS. 29A-29B. The clustered human transcriptomic data showed clustering by slide (FIG. 29A) i.e., by one or more AOIs of the same slide, and tissue type (FIG. 29B).

Validation of Buffa, Winter, Ragnum, and ssGSEA Hypoxia Scores

Hypoxia scores for each AOI across all cancer samples were determined using multiple gene sets associated with human hypoxic signaling (i.e., genes that are overexpressed during hypoxia). Numerical hypoxia scores were calculated by comparing each normalized gene's abundance within every AOI to its median across all AOIs while subsetting to genes within the respective gene sets (e.g., Buffa, Winter, Ragnum), such that abundances above the median are awarded +1 and below the median are awarded −1, herein called the “median rank sum” method. The Buffa hypoxia gene set was described by Buffa et al. 2010 Br. J. Cancer; the Winter hypoxia gene set was described by Winter et al. 2007 Cancer Res; and the Ragnum hypoxia gene set was described by Ragnum et al. 2015 Br. J. Cancer. The genes within these get sets were further subset to those found on the GeoMx's Cancer Transcriptome Atlas (CTA), which comprises probes against approximately 1800 genes, for a total of 16 genes in the Buffa gene set, 20 genes in the Winter gene set, and 10 genes in the Ragnum gene set known to be correlated with tissue hypoxia. The intersecting Buffa genes included the following: ADM, ANLN, DDIT4, ENO1, HK2, LDHA, P4HA1, SLC16A1, SLC2A1, VEGFA, GPI, TPII, MAD2L2, ALDOA, BNIP3, MIF. The intersecting Winter genes included the following: ANGPTL4, ANLN, COL4A5, LDHA, MNATI, P4HA1, PLAU, PSMB7, PVR, SLC16A1, SLC2A1, VEGFA, TPII, CXCL8, NDUFA4L2, PGF, ALDOA, BNIP3, KRT17, MIF. The intersecting Ragnum genes included the following: ADM, BIRC5, DDIT4, DSP, FOXMI, G6PD, MCM2, P4HA1, PKM, RIMKLA. The median rank sum method determine the hypoxia score from equation (1) shown below:

$$hypoxia score ( RNA ) = {\sum }_{i = 1}^{signature size} f ( x ) = \{ \begin{matrix}+ 1 , {g}_{i} ( x ) > median ( {g}_{i} ( x ) ) \\ - 1 , {g}_{i} ( x ) < medium ( {g}_{i} ( x ) )\end{matrix} \qquad ( 1 )$$

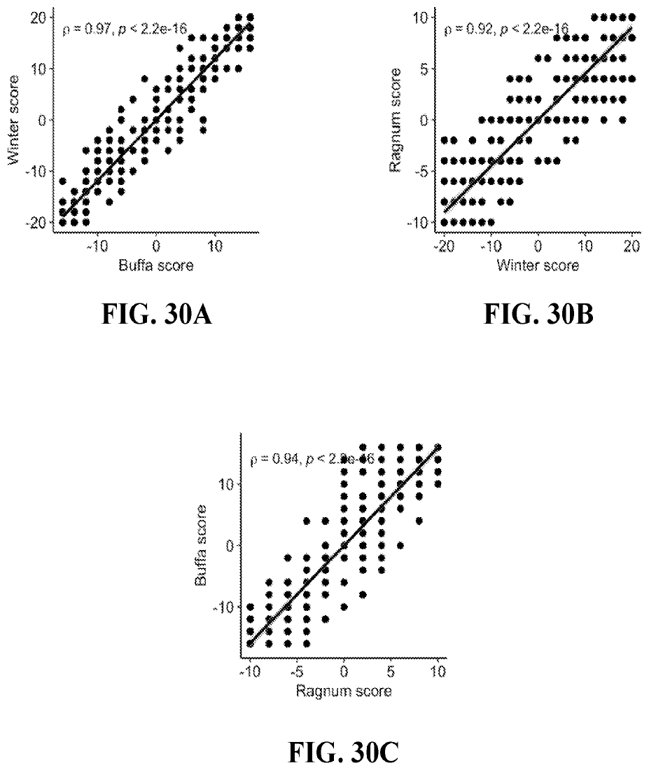

Specifically, the median expression of each gene of a gene set across all AOIs is determined and used as a comparison to the individual AOI's gene expression of the particular gene gᵢ(x) of the gene set. If the normalized expression of sequencing reads for a particular gene in an AOI is greater than the median normalized expression of the gene across all of the AOIs, then the hypoxic score for a given AOI is increased by 1. If the normalized expression of sequencing reads for a particular gene in an AOI is less than the median normalized expression for the given gene, then the hypoxia score is decreased by 1. The above is repeated for all genes of a particular set and a resulting hypoxia score is determined using the Buffa, Winter, or Ragnum gene sets, or any other similar hypoxia-associated gene set. From FIGS. 30A-30C we can see that the hypoxia scores generated using all three gene sets are highly correlated between Winter and Buffa (FIG. 30A), Ragnum and Winter (FIG. 30B), and Buffa and Ragnum (FIG. 30C).

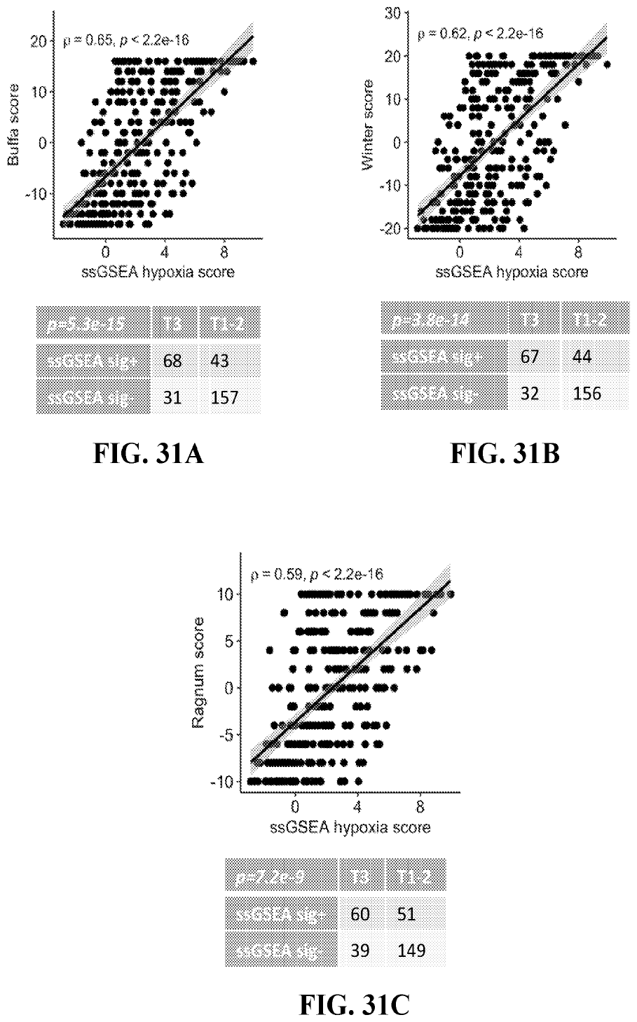

Additionally a binary classifier, single-sample gene set enrichment analysis (ssGSEA), was used to independently validate the Buffa, Winter, and Ragnum hypoxia scores derived from normalized human gene expression. ssGSEA provides a binary determination of whether a given AOI of a sample is significantly enriched, i.e., genes in the ssGSEA gene set are coordinately up regulated in the given AOI. ssGSEA then provides a quantitative enrichment score (“ES”), statistical significance of ES based on ES_{Null} from permuted data (p-value) that indicates if a given AOI is significantly enriched in a given ssGSEA gene set, and FDR-corrected significance (q-value). Various ssGSEA gene sets exist, including one for hypoxia, of which 60 genes intersected with the GeoMx's CTA human gene expression data. FIGS. 31A-31C show correlation between ssGSEA hypoxia enrichment score versus each of Buffa (FIG. 31A), Winter (FIG. 31B), and Ragnum (FIG. 31C) scores. From FIGS. 31A-31C, it can be seen that Buffa, Winter, and Ragnum gene sets are strongly correlated to ssGSEA hypoxia scores. Chi-squared (X²) analysis of the significantly enriched AOIs determined from the ssGSEA's q-value output were compared between an upper third tertile and lower third tertile in combination with the middle third tertile of Buffa, Winter, and Ragnum hypoxic scores. The X² test results shown in FIGS. 31A-31C indicate that the AOIs comprising the upper third tertile of hypoxia scores were significantly enriched in AOIs containing significantly enriched hypoxia by ssGSEA. These results indicate that the AOIs comprising the upper third tertile of Buffa, Winter, and Ragnum hypoxia scores can be considered as phenotypically hypoxic.

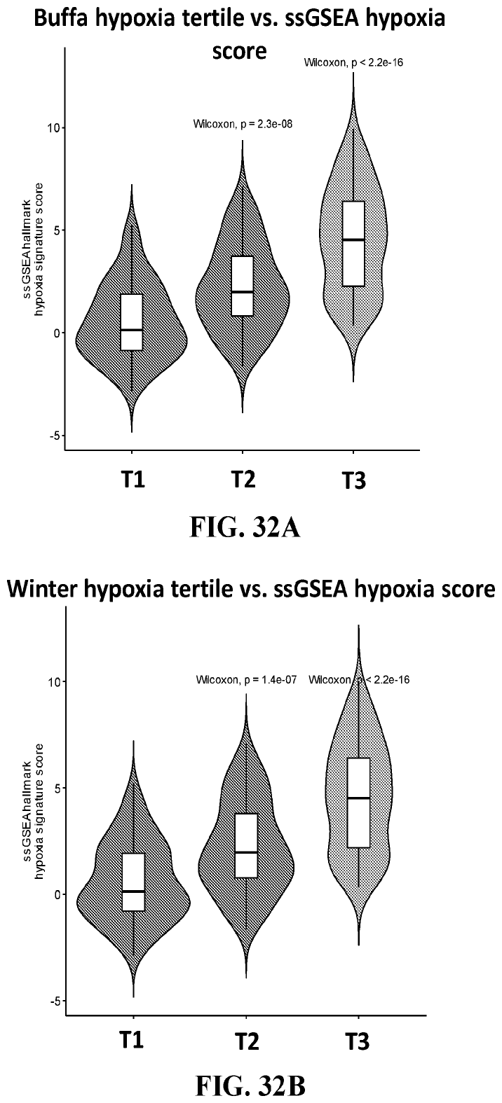

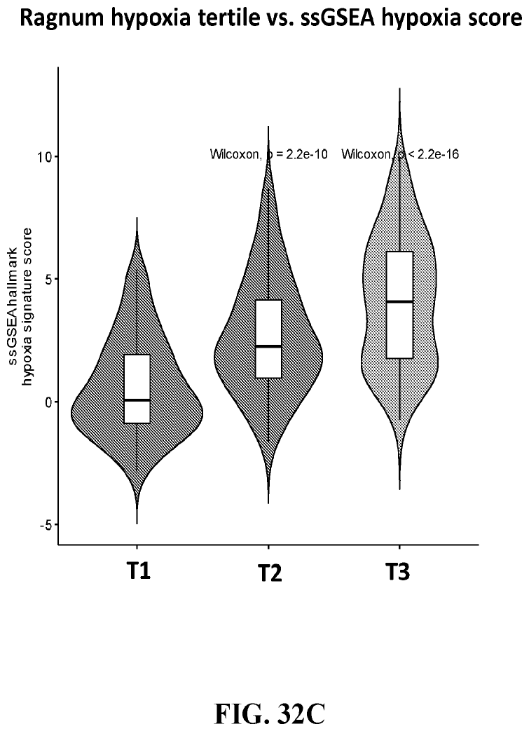

FIGS. 32A-32C show the ssGSEA hallmark hypoxia signature score compared against the first, second, and third tertile of the Buffa (FIG. 32A), Winter (FIG. 32B), and Ragnum (FIG. 32C) hypoxia scores. The stepwise increase in ssGSEA enrichment scores between the various tertiles of the Buffa, Winter, and Ragnum hypoxia scores shown in FIGS. 32A-32C agrees with the results shown in FIGS. 31A-31C.

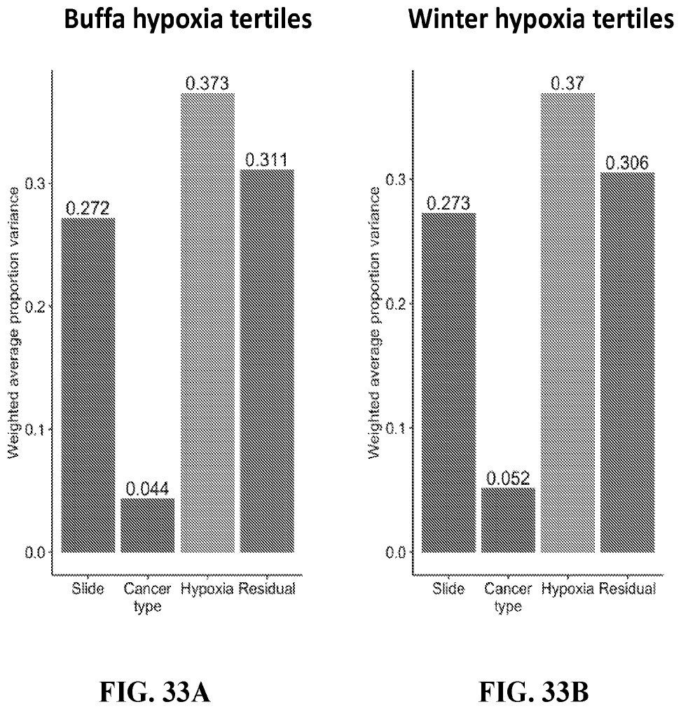

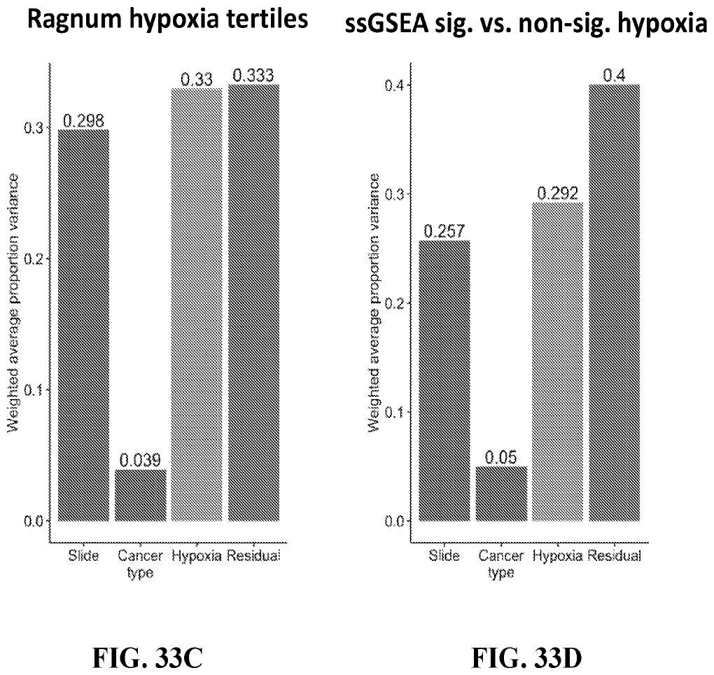

Principal variance component analysis (PVCA) estimates the amount of variance in the data that is attributable to any factor of the data, e.g., the slide, cancer type, amount of hypoxia, or a residual. From the PVCA plots for Buffa (FIG. 33A), Winter (FIG. 33B), Ragnum (FIG. 33C), and ssGSEA (FIG. 33D), it can be seen that hypoxia was the dominating factor when compared to the other factors in explaining the variance in the normalized human gene expression data.

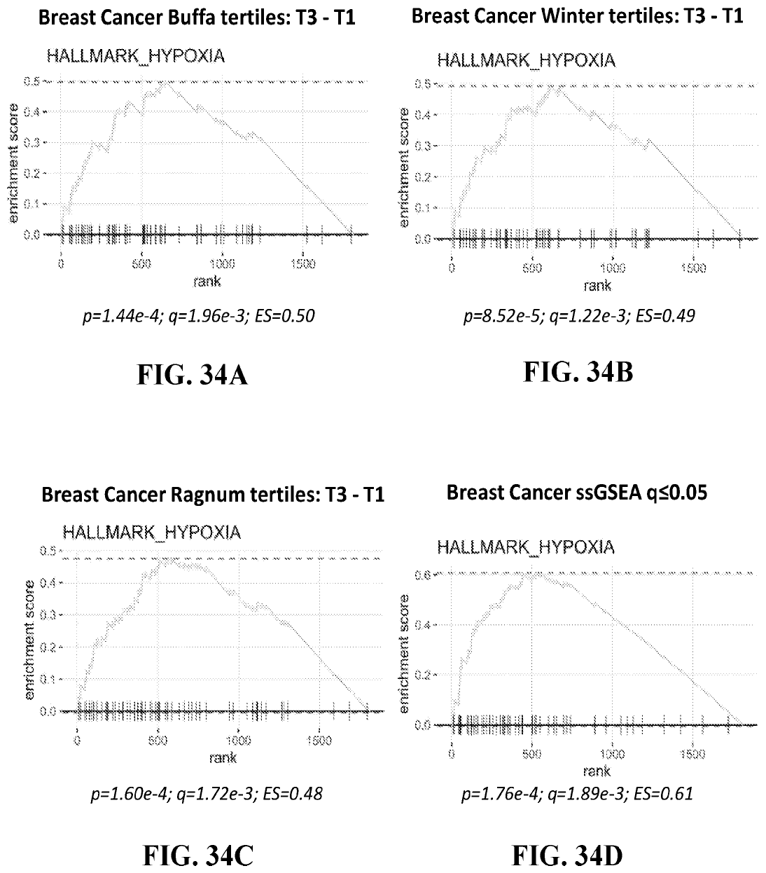

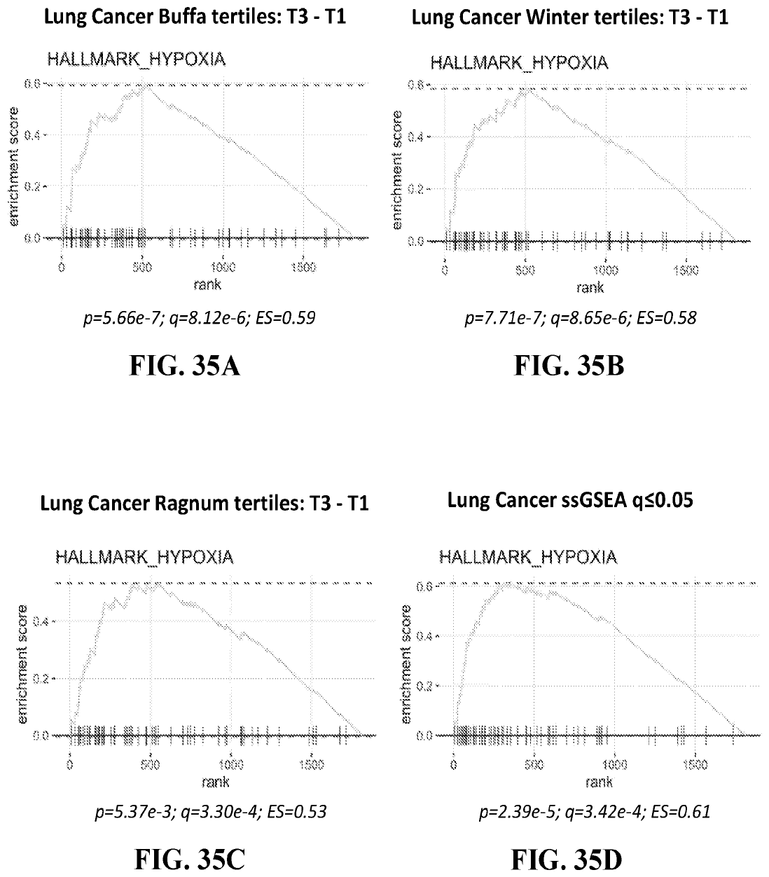

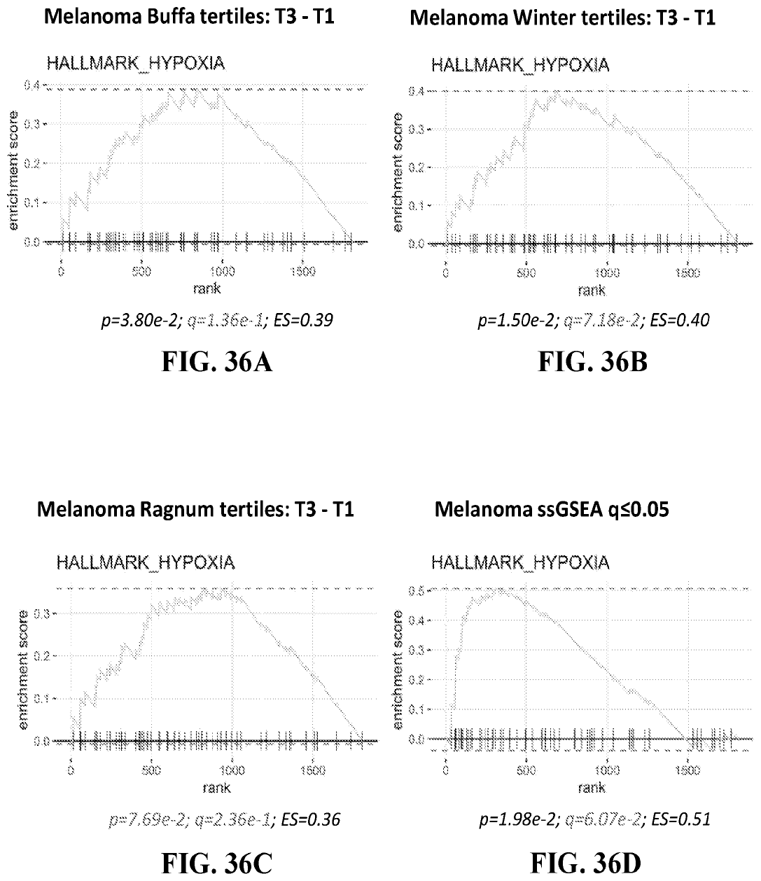

Per-tissue local gene expression changes between hypoxic and normoxic AOIs were then analyzed for concordance to the global gene expression differences expected for hypoxic and normoxic AOIs. The steps involved to determine the concordance included: determining a normalized gene expression for each AOI and associated hypoxic tertile for each AOI based on Buffa, Winter, and Ragnum gene sets, or binary hypoxic enrichment using ssGSEA gene sets; providing the hypoxic tertile or binary classes per AOI per cancer type to a linear mixed model (LMM) to determine differential gene expression among samples that are related to each other (e.g., AOIs from the same slide); determining which genes between hypoxic categories are differentially expressed between them per cancer type; and feed the global differential gene list to fast GSEA (FGSEA) analysis on a per cancer type basis to determine whether the local hypoxic scores truly reflect expected global hypoxic changes. From FIGS. 34A-34D, one can see that local gene expression in breast cancer tissue samples associated with Buffa (FIG. 34A), Winter (FIG. 34B), and Ragnum (FIG. 34C) scores between the third and first tertiles, or binary ssGSEA classes (FIG. 34D), provide global differentially expressed genes that are significantly enriched in the FGSEA hypoxic gene set. Similarly for lung cancer tissues samples, local gene expression contributing to Buffa (FIG. 35A), Winter (FIG. 35B), and Ragnum (FIG. 35C) scores between the third and first tertiles, or binary ssGSEA classes (FIG. 35D), provide globally differentially expressed genes that are significantly enriched in the FGSEA hypoxic gene set. Lastly, local-global gene expression concordance was borderline for melanoma cancer tissue samples, as shown for Buffa (FIG. 36A), Winter (FIG. 36B), Ragnum (FIG. 36C), and ssGSEA (FIG. 36D) tertiles or binary classes, respectively.

### Microbial Alpha Diversity of AOIs

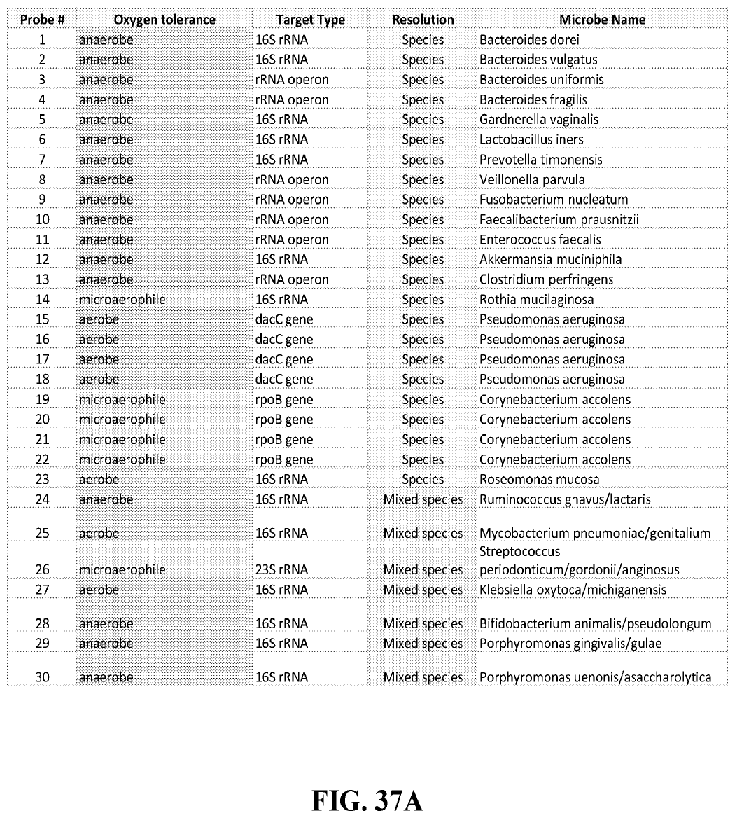

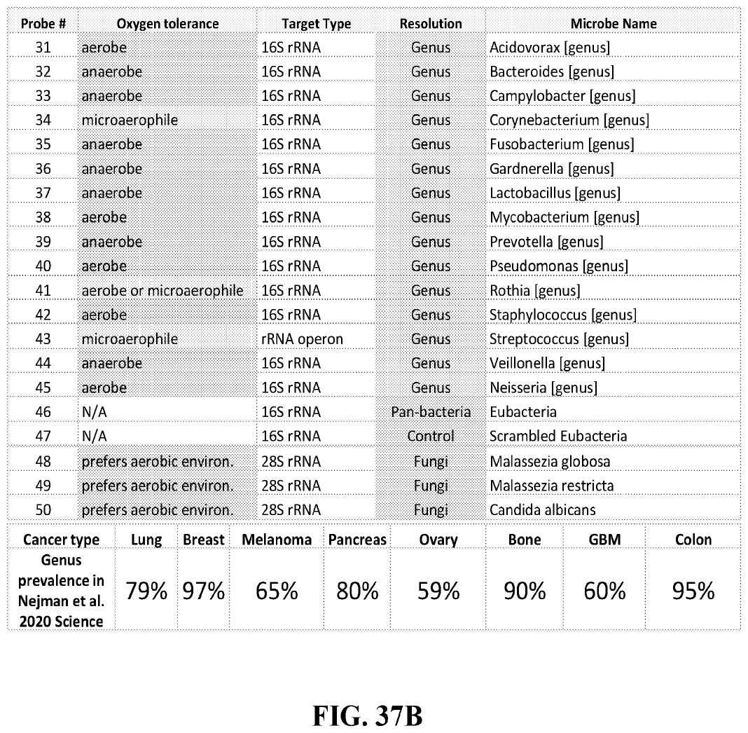

A custom panel of microbial targets with corresponding probes targeting microbial RNA, as shown in FIGS. 37A-37B, was used in the spatial RNA transcriptomic analysis to determine the presence of microbes in an AOI. The probes, as shown in FIGS. 37A-37B, target microbial 16S rRNA nucleic acid molecule sequences and/or specific microbial target genes. As a negative control, a scrambled eubacterial target was used, as shown in FIG. 37B.

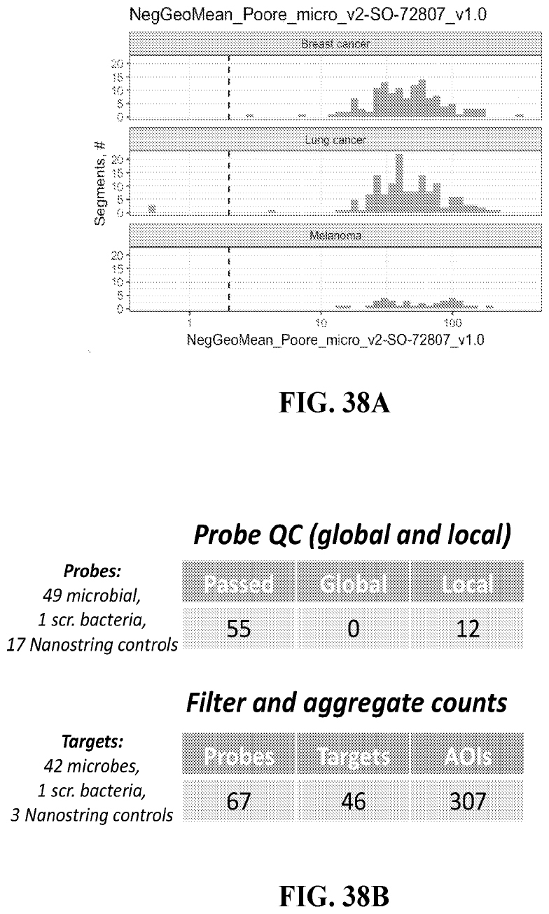

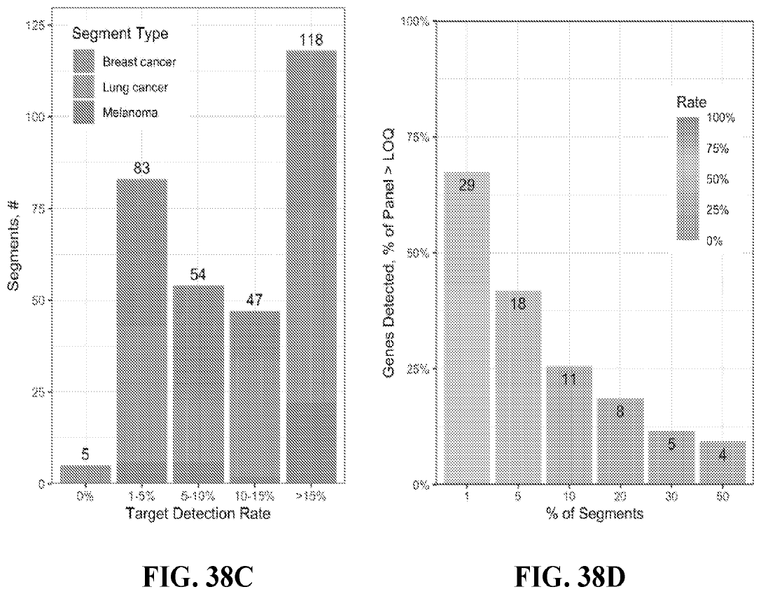

Quality control results for microbial sequences and microbial probe-QC, as shown FIGS. 38A-38B, indicate that the microbial sequences and the corresponding probe-QC pass the required QC metrics and thus are valid to use in further downstream analysis. FIGS. 38C-38D show the number of AOIs (i.e., segments) vs. target detection rate of microbes across tissue type (FIG. 38C), and percentage of AOIs vs. percent of targeted microbes above the limit of quantification filter (FIG. 38D), as described in some embodiments herein.

Limit of quantification (LOQ) filtering was used to determine the “noise” threshold of bacterial targets detection per AOI based on the presence of eubacterial probe in 6 tissue-adjacent paraffin AOIs. In other embodiments, the paraffin could be exchanged for another negative control sample or region, including but not limited to germ-free mouse tissue, sterile cell pellets, or other experimental contamination control types. Limit of quantification filtering is defined by equation (2) shown below:

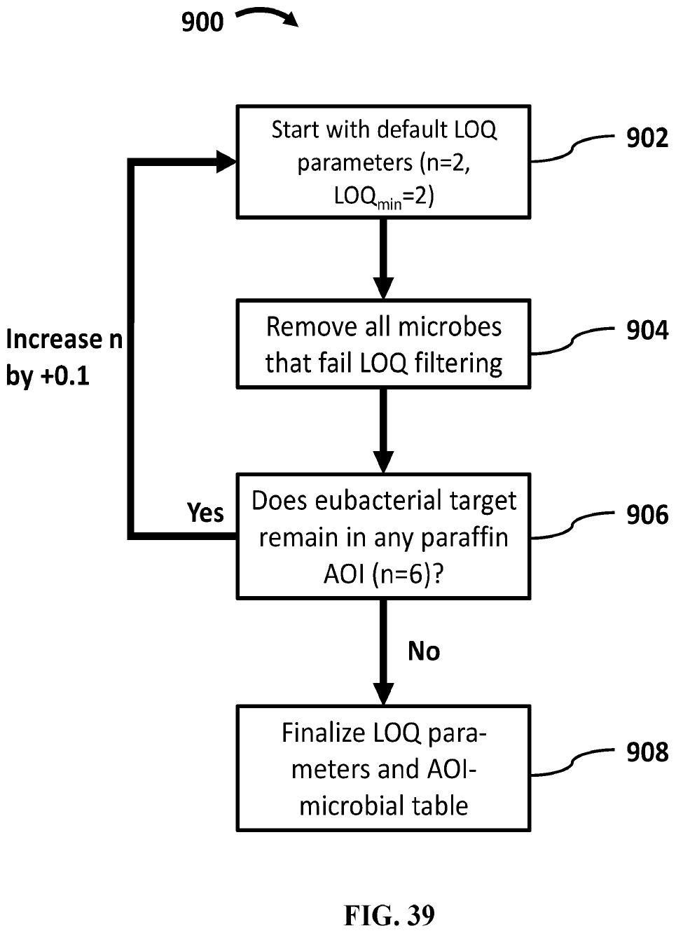

LOQᵢ=geoMean(NegProbeᵢ)*geoSD(NegProbᵢ)ⁿ (2)

where the NegProbeᵢ is the expression of the scrambled eubacterial probe, and n is a tunable hyperparameter. A tuned hyperparameter, n, for LOQ filtering across all AOIs was determined by analyzing the expression of the eubacterial probe in the 6 tissue-adjacent paraffin AOIs. The method 900 to determine the tuned hyperparameter, n, can be seen in FIG. 39. The method 900 included the following: providing a default manufacturer's setting for the hyperparameter, n=2, and LOQₘᵢₙ=2 (902); removing all microbial targets that fail LOQ filtering; determining if the microbial eubacterial probe target remains in any paraffin AOI 906; if the eubacterial probe target remains, then n is increased by 0.1; if the eubacterial probe target does not remain, then the LOQ n hyperparameter and per-AOI microbial table are finalized 908. Through the method 900, a hyperparameter, n=2.7, was determined as the threshold hyperparameter that filtered out eubacterial target hits in all off-tissue paraffin AOIs; thus, when applied across all AOIs, the hyperparameter enabled per-AOI noise threshold estimation, such that per-AOI microbial target expression is only retained (or detected) when above the per-AOI, paraffin-induced noise floor.

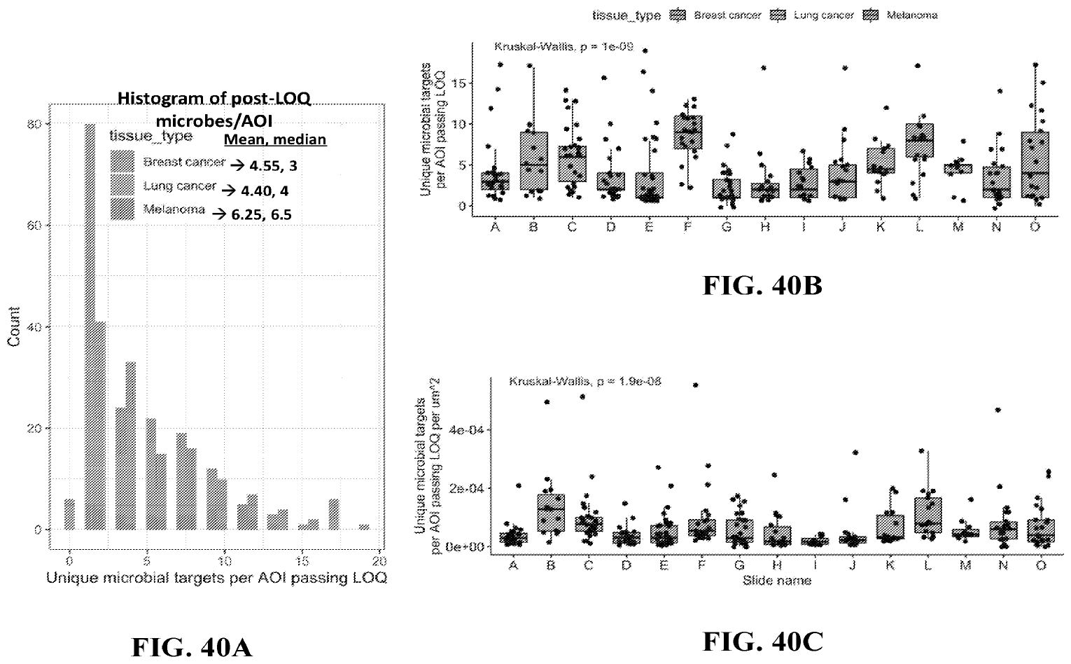

FIGS. 40A-40C show the number of unique microbial targets post-LOQ filtering across tissue types (FIG. 40A), and across tissue type and slide (FIG. 40B). Since the AOIs vary in size from one another, the number of microbial target hits post-LOQ filter was normalized by the area of each AOI, as shown in FIG. 40C.

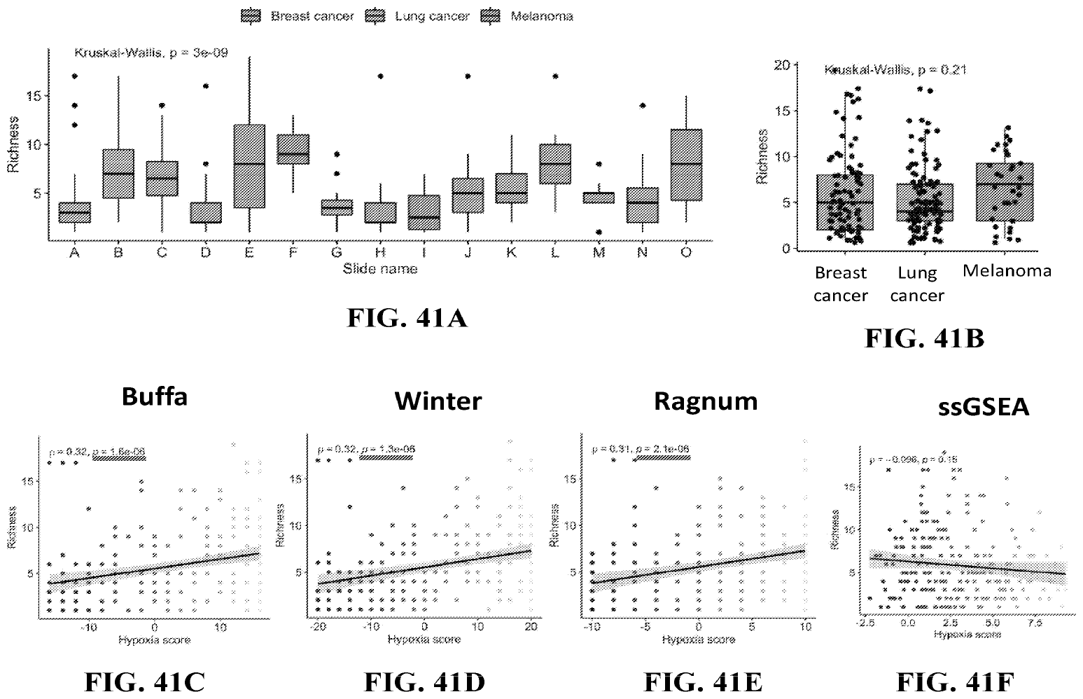

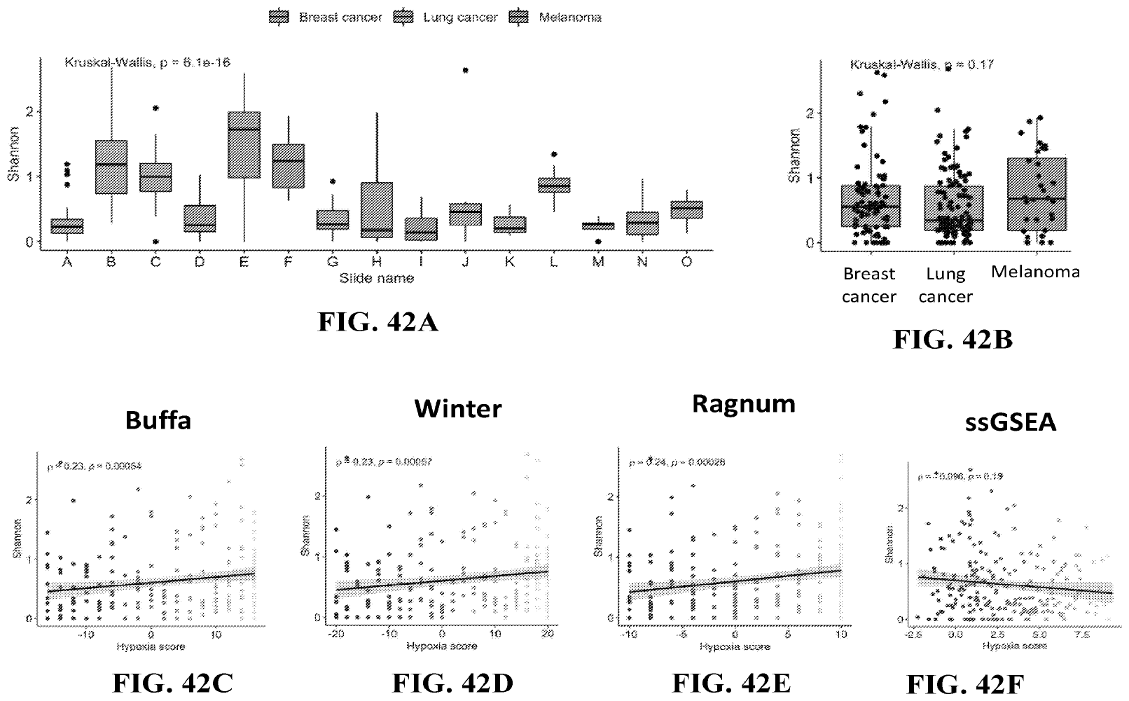

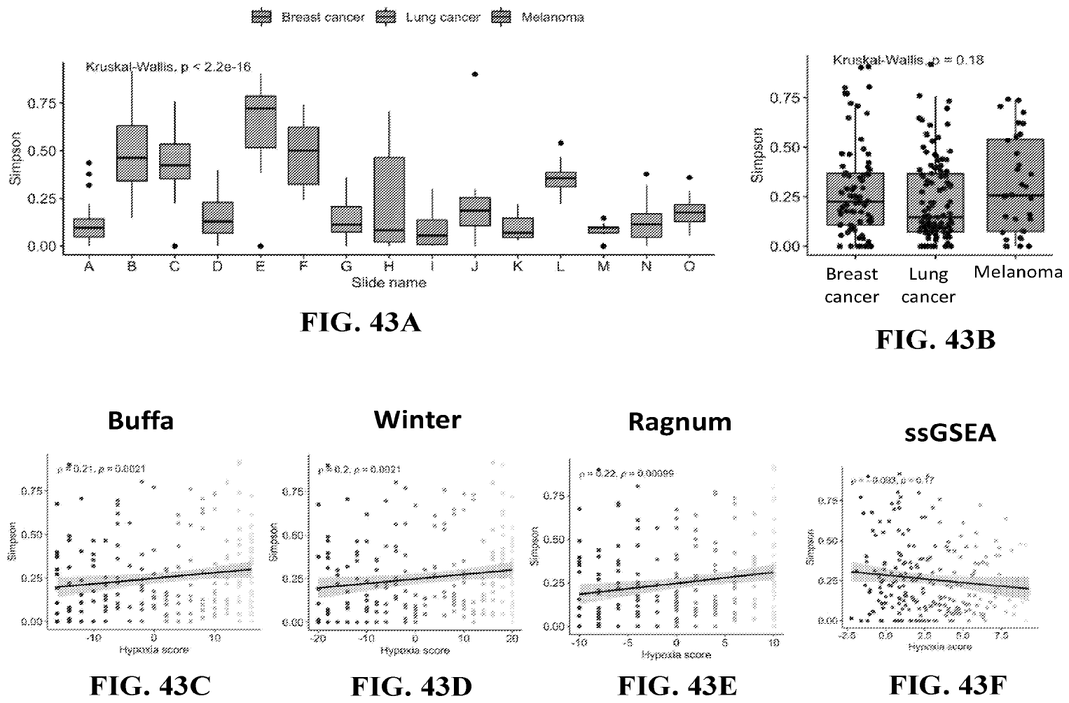

Alpha diversity of rarefied microbial target sequence reads were analyzed across tissue type and slide, as shown in FIGS. 41A-41F. It was found that alpha diversity (richness) varied substantially across slide or sample (FIG. 41A) and varied lesser across tissue type (FIG. 41B). Additionally, alpha diversity was significantly correlated to Buffa (FIG. 41C), Winter (FIG. 41D), and Ragnum (FIG. 41E) hypoxia scores, indicating that the more hypoxic an AOI is, the more diverse the microbial community becomes. ssGSEA hypoxia scores were not found to be correlated with alpha diversity, as shown in FIG. 41F. In addition to richness, Shannon entropy (FIGS. 42A-42F), and the Simpson index (FIGS. 43A-43F) measures of alpha diversity detected across tissue type and sample slide were also analyzed. Similar to richness, Shannon entropy and Simpson index were found to vary substantially across slides (FIGS. 42A and 43A), vary lesser across tissue type (FIGS. 42B and 43B), and were found to be correlated to Buffa (FIGS. 42C and 43C), Winter (FIGS. 42D and 43D), and Ragnum (FIGS. 42E and 43E) hypoxia scores. Shannon entropy and Simpson index were also found to not be correlated to ssGSEA hypoxia scores, as shown in FIGS. 42F and 43F.

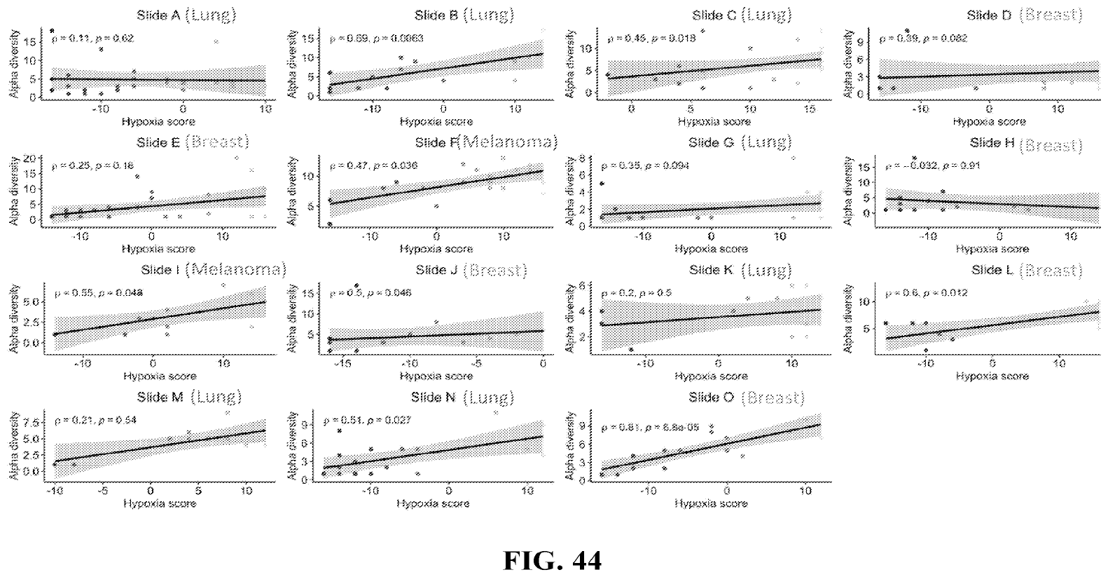

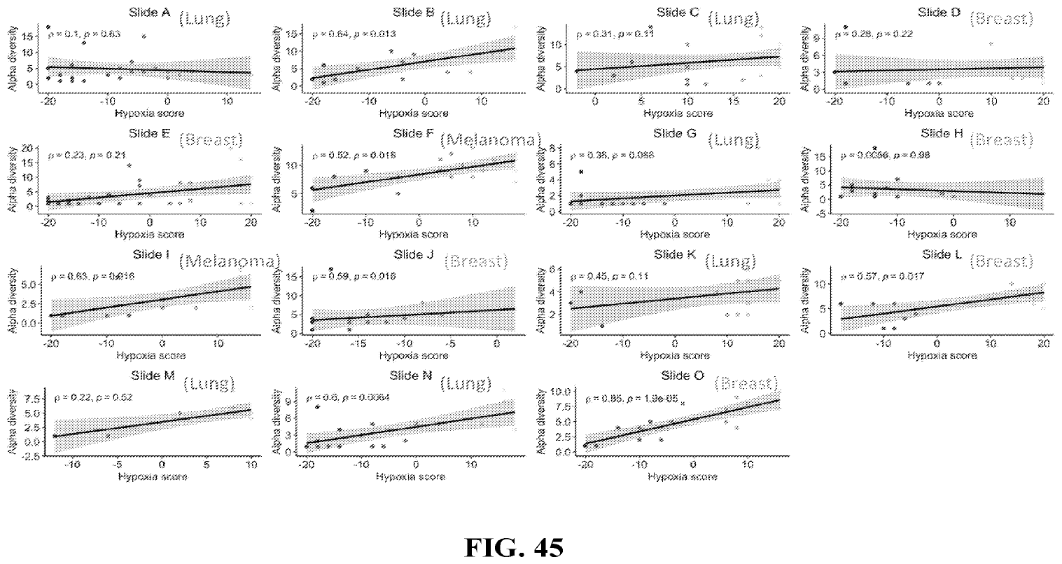

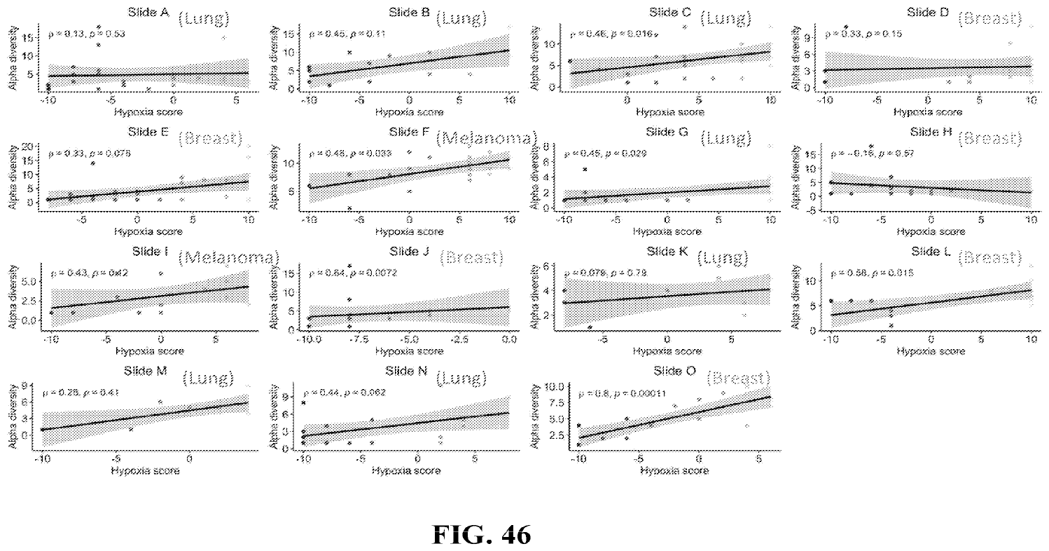

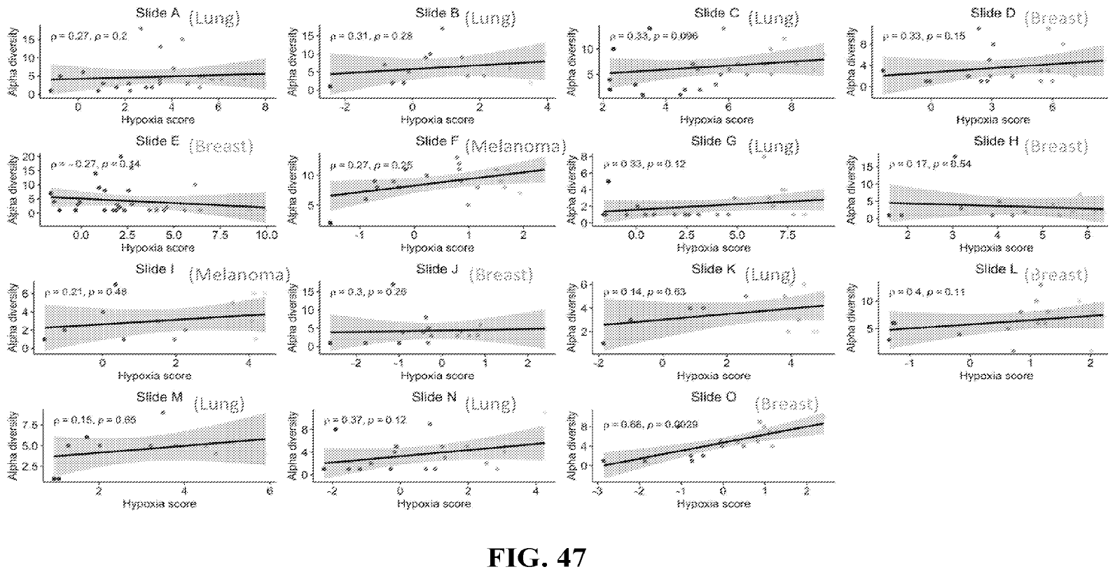

The correlation between alpha diversity and the Buffa, Winter, Ragnum, and ssGSEA hypoxia scores was also analyzed in an intra-slide or intra-sample basis. LOQ-filtered, non-normalized, microbial counts were rarefied for each slide before calculating alpha diversity. To maximize retention of AOIs per slide, rarefaction was done using either (i) minimum per-slide AOI read count rounded to the nearest 100 or (ii) rounded to 50 reads if the result of (i) is 0. The alpha diversity of each AOI of a given slide was plotted against Buffa (FIG. 44), Winter (FIG. 45), Ragnum (FIG. 46), and ssGSEA (FIG. 47) hypoxia scores. Increases in hypoxia scores of Buffa across 8 of 15 slides, Winter across 7 of 15 slides, Ragnum across 6 of 15 slides, and ssGSEA across 1 of 15 slides were found to significantly correlate to increases in alpha diversity. All Spearman correlations that were statistically significant were positive in direction.

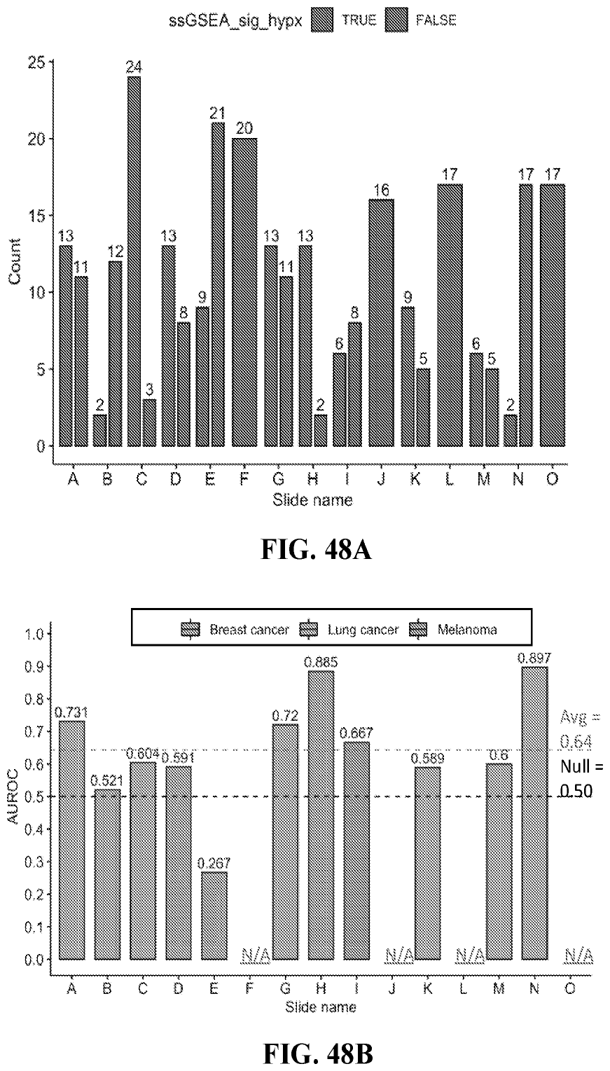

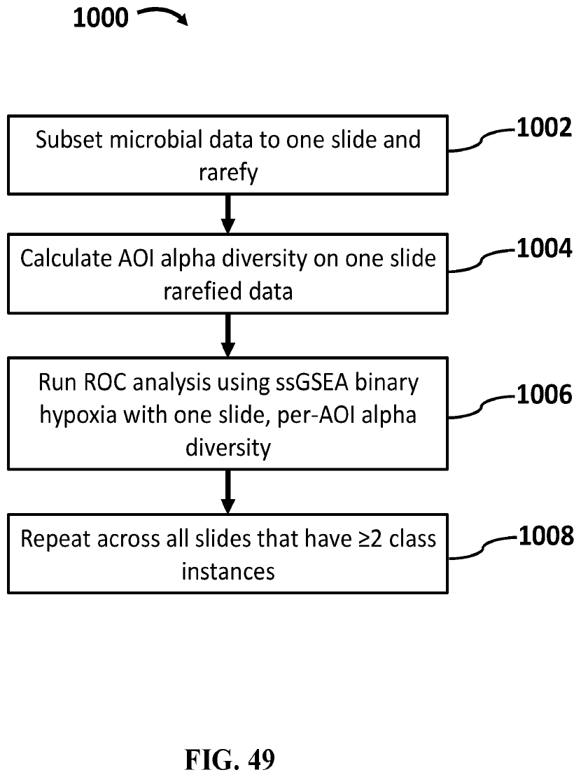

Intra-slide alpha diversity was then tested as a classifier in predicting hypoxia or normoxia based on binary ssGSEA hypoxic scores. ssGSEA hypoxic scores based on the ssGSEA q-values was determine for each slide's AOIs, as shown in FIG. 48A. Corresponding microbial alpha diversity for each slide was then calculated using the method 1000, as shown in FIG. 49. The method was comprised of the following steps: (a) subset LOQ-filtered, non-normalized microbial count data to a single slide and rarefy 1002; (b) calculate per-AOI alpha diversity across all AOIs for a slide from the rarefied microbial count data 1004; (c) run receiver operating characteristic (ROC) analysis using ssGSEA binary hypoxia with one slide, per-AOI alpha diversity 1006; and repeat steps (a)-(d) across all slides that have greater than or equal to 2 class ssGSEA class instances. The resulting predictive performance of the alpha diversity classifier per slide is shown in FIG. 48B. The average area under the ROC curve (AUROC) across all slides was 0.64, indicating that per slide alpha-diversity classifier of hypoxia is a moderate classifier.

### Microbial Beta Diversity Across Slides

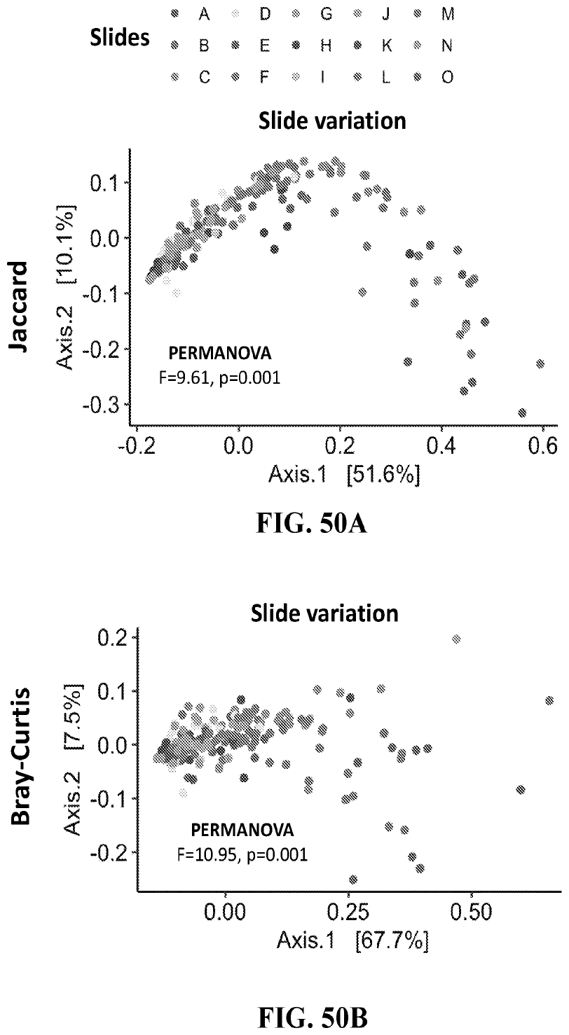

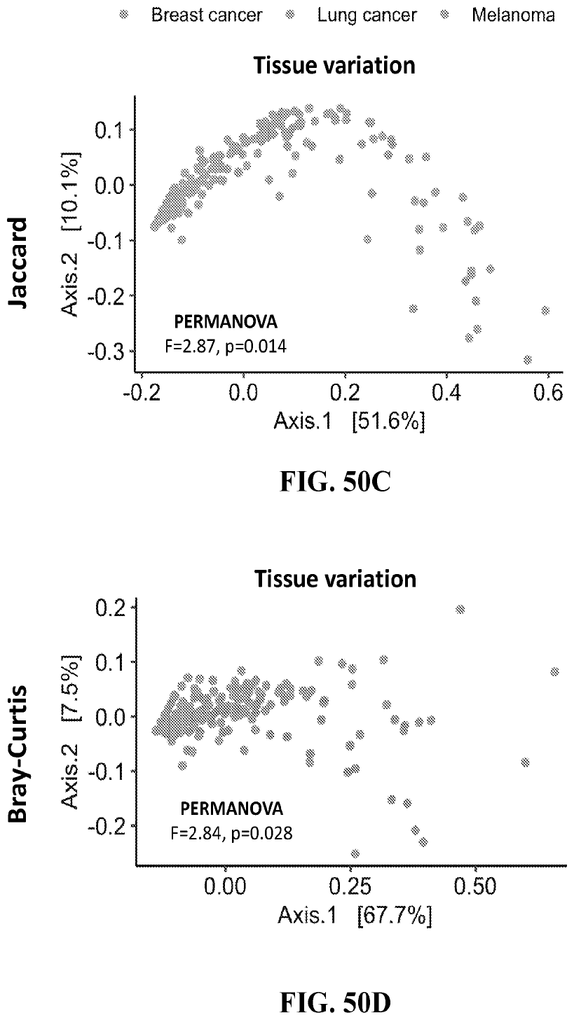

Beta diversity is a measure of the difference in microbial target composition between AOIs. Slide variation (FIGS. 50A-50B) and tissue variation (FIGS. 50C-50D) factors were analyzed across all slides using permutational multivariate analysis of variance (PERMANOVA) to determine the significance of either factor to the variance observed in the data. A brief description of the PERMANOVA analysis methods follows. LOQ-filtered, non-normalized pan-AOI, microbial counts were rarefied to the first quartile (560 reads) before calculating alpha diversity. The “Eubacteria” positive control probe was removed prior to analysis. Jaccard (FIGS. 50A and 50C) and Bray-Curtis (FIGS. 50B and 50D) distances were calculated using the rarefied table. PERMANOVA was then calculated using adonis2 in R with 999 permutations, providing a Pseudo-F statistic and associated p-value, shown inset on the plots shown in FIGS. 50A-50D. Jaccard distance calculations are solely based on presence-absence whereas Bray-Curtis distance calculations are weighted by microbes' relative abundances. From the PERMANOVA analysis shown in FIGS. 50A-50D, differences in slide appear to explain more variance in the microbial composition than that explained by tissue type variation.

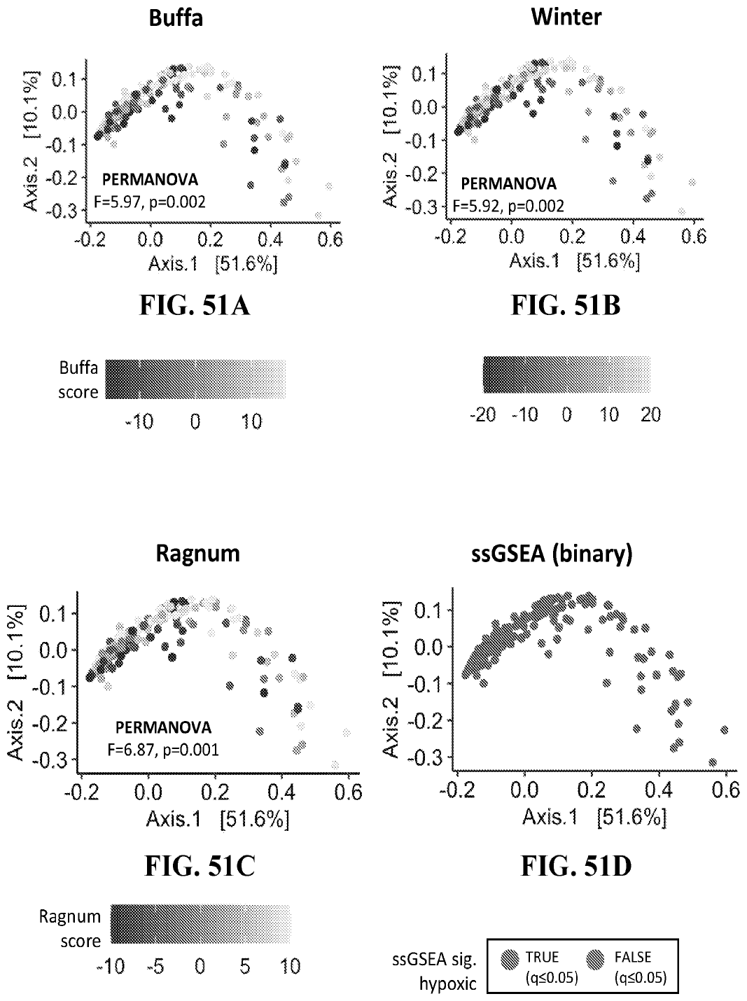

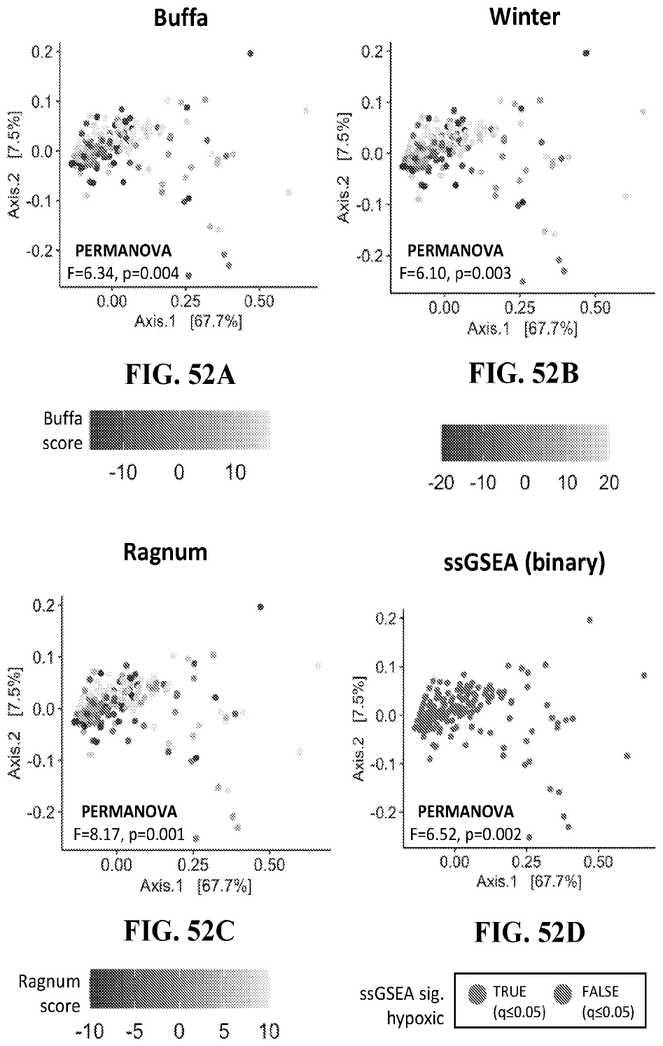

Jaccard (FIGS. 51A-51D) and Bray-Curtis (FIGS. 52A-52D) PERMANOVA analyses were also conducted across all AOIs with factors of Buffa (FIGS. 51A and 52A), Winter (FIGS. 51B and 52B), and Ragnum hypoxia scores (FIGS. 51C and 52C), or ssGSEA binary hypoxic category (FIGS. 51D and 52D). On a global scale, there is significant variation of microbial composition within the AOIs on the basis of hypoxia regardless of how hypoxia is calculated.

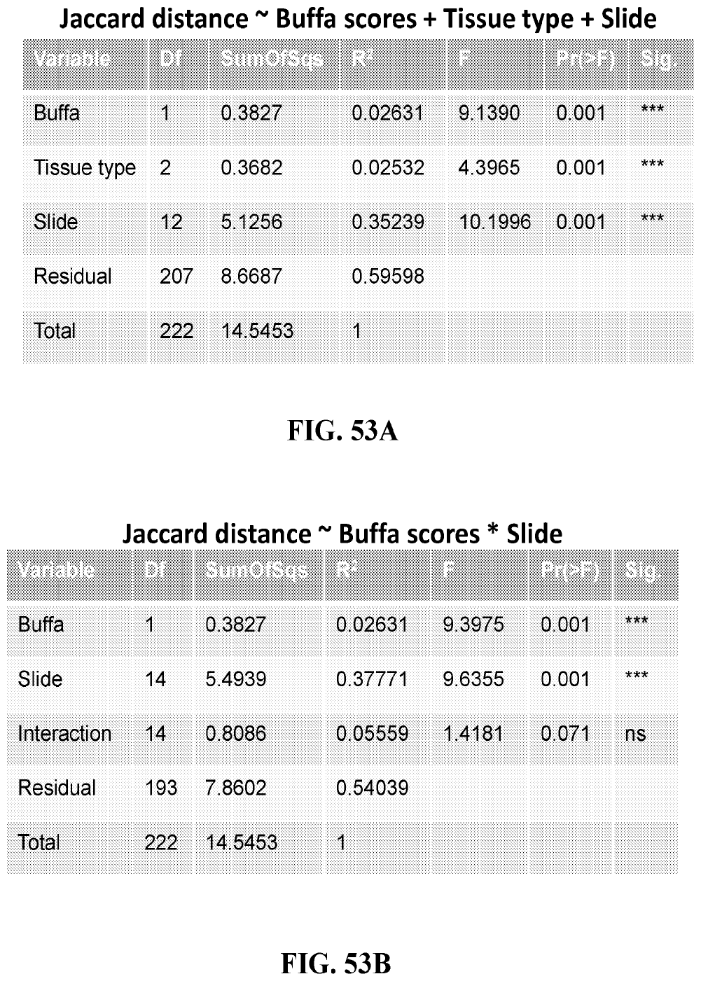

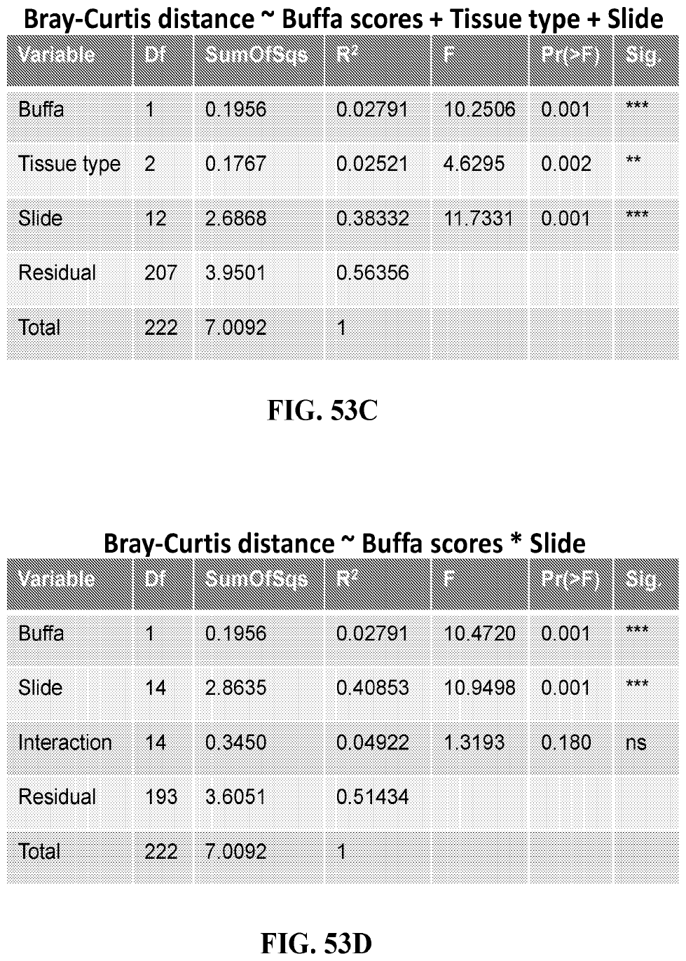

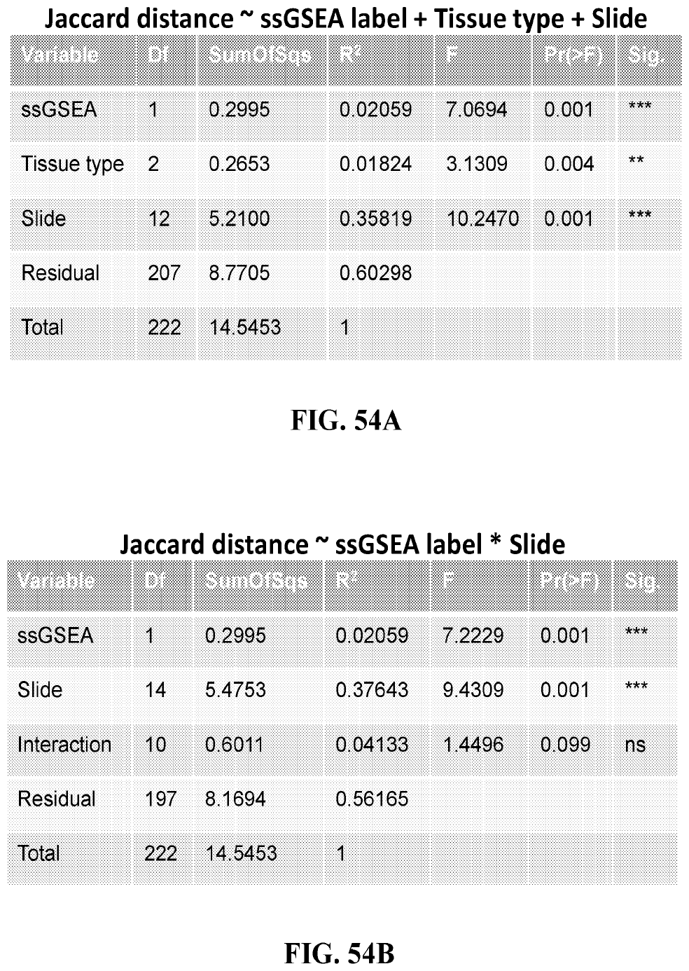

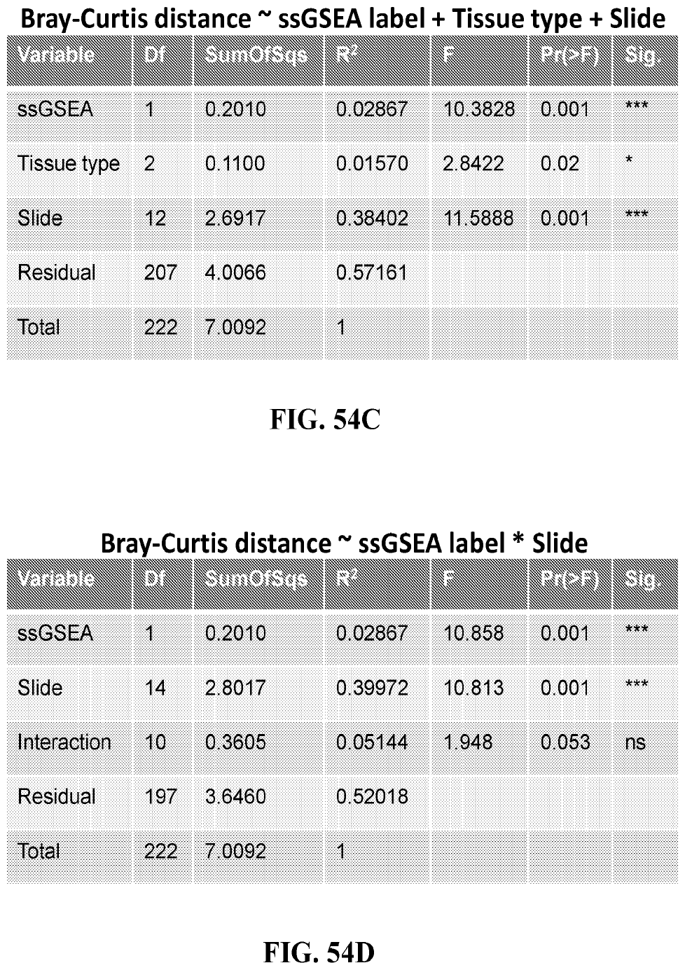

Multivariate Jaccard (FIGS. 53A-53B and 54A-54B) and Bray-Curtis (FIGS. 53C-53D and 54C-54D) PERMANOVA analyses, taking into consideration factors of Buffa hypoxia scores (FIGS. 53A-53D), ssGSEA binary hypoxic category (FIGS. 54A-54D), tissue type, slide, and interactions thereof, were then completed. From the multivariate PERMANOVA analyses, it can be seen that Buffa hypoxic scores, ssGSEA binary hypoxic category, slide, and tissue type, individually represent significant variation in the data distribution. However, the interaction between the slide and the hypoxia scores was not significant for any of these analyses, suggesting that it is a pan-slide, pan-cancer type effect.

Machine Learning and Regression with Global Microbial Compositions to Predict Hypoxia

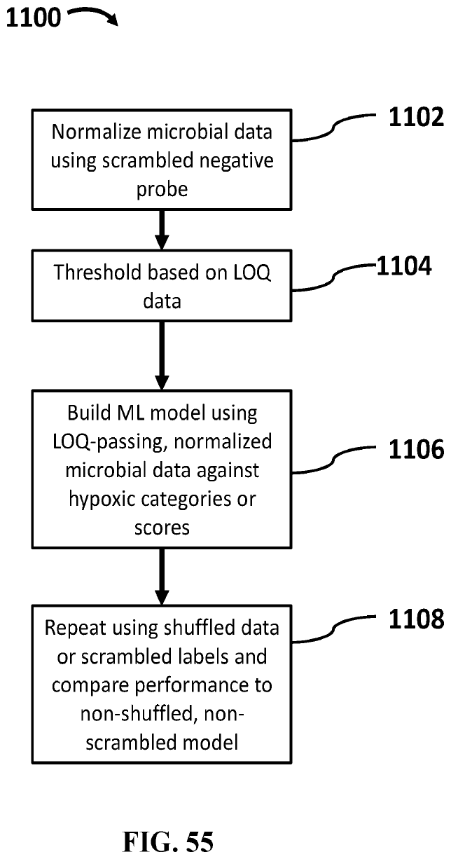

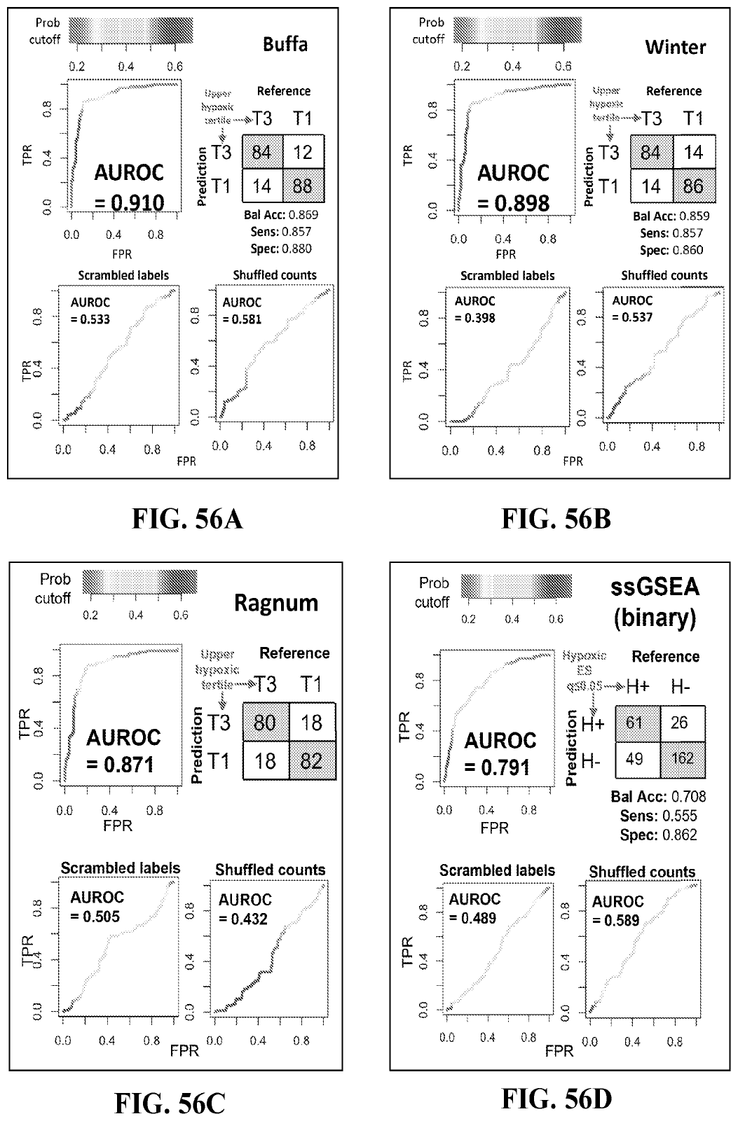

Next, the feasibility of using machine learning with the global, LOQ-filtered, normalized microbial data to predict the hypoxic AOIs of each slide based on the slide's microbial composition was analyzed, as shown in FIGS. 56A-56D. The method 1100, as shown in FIG. 55, of building the machine learning models using global microbial data included the steps of: (a) normalizing the microbial data using the scrambled eubacterial probe 1102; (b) thresholding the normalized microbial data using LOQ filtering 1104; (c) training a gradient boosting machine model with the LOQ thresholded microbial abundance and hypoxic score labels using leave-one-out-cross-validation (LOOCV); (d) and repeating (c) with shuffled data or scrambled labels as negative control analyses. The resulting ROC, AUROC, scrambled AUROC, and shuffled count AUROC for each of the trained models is shown for Buffa (FIG. 56A), Winter (FIG. 56B), Ragnum (FIG. 56C), and ssGSEA (FIG. 56D) hypoxia score labels. For Buffa, Winter, and Ragnum models, AOIs comprising the upper and lower tertiles of scores were compared; for ssGSEA models, AOIs were categorized as either hypoxic or non-hypoxic based on the ssGSEA-calculated q-value. The top five microbial features for the Buffa-labeled, trained gradient boosting machine model were genus level Pseudomonas, Faecalibacterium prausnitzii, genus level Bacteroides, genus level Neisseria, genus level Mycobacterium, and genus level Corynebacterium. The top five microbial features for the Winter-labeled, trained gradient boosting machine model were genus level Pseudomonas, Faecalibacterium prausnitzii, genus level Corynebacterium, genus level Bacteroides, Eubacteria (pan-bacteria), and genus level Mycobacterium. The top five microbial features for the Ragnum-labeled, trained gradient boosting machine model were genus level Pseudomonas, Faecalibacterium prausnitzii, genus level Corynebacterium, genus level Neisseria, genus level Bacteroides, and genus level Mycobacterium. The top five microbial features for ssGSEA-labeled, trained gradient boosting machine model were Faecalibacterium prausnitzii, genus level Bacteroides, genus level Corynebacterium, genus level Pseudomonas, Eubacteria (pan-bacteria), and genus level Mycobacterium. In all instances, the negative control analyses using scrambled metadata labels or shuffled count data produced approximately random classifiers (AUROC ˜=0.5).

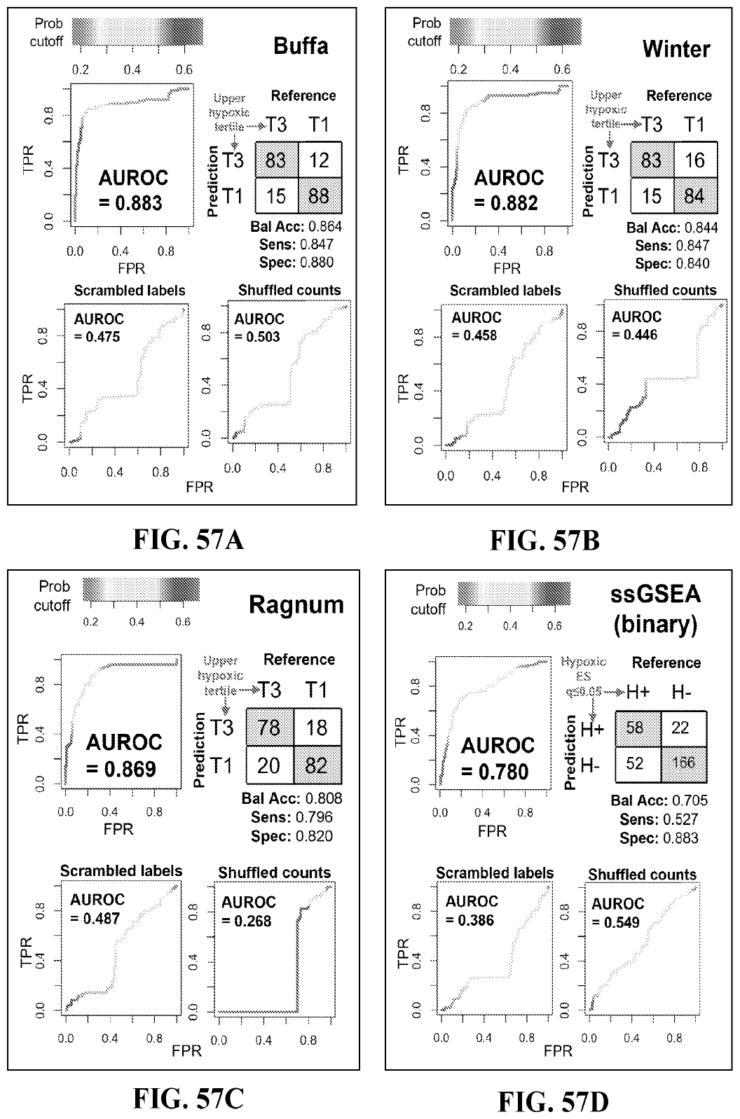

The aforementioned machine learning training was replicated with regularized logistic regression machine learning model with the ROC and AUROC results shown in FIGS. 57A-57D. The observed performance of the regularized logistic regression machine learning model is similar to but slightly weaker than the gradient boosting machines shown in FIGS. 56A-56D. The top five microbial features for the Buffa-labeled, trained regularized logistic regression model were genus level Pseudomonas, genus level Corynebacterium, genus level Prevotella, Rothia mucilaginosa, Eubacteria (pan-bacteria), and genus level Bacteroides. The top five microbial features for the Winter-labeled, trained regularized logistic regression model were genus level Pseudomonas, genus level Corynebacterium, genus level Neisseria, Eubacteria (pan-bacteria), genus level Prevotella, and genus level Mycobacterium. The top five microbial features for the Ragnum-labeled, trained regularized logistic regression model were genus level Pseudomonas, genus level Corynebacterium, genus level Neisseria, genus level Mycobacterium, Mycobacterium pneumoniae/genitalium, and Faecalibacterium prausnitzii. The top five microbial features for ssGSEA-labeled, trained regularized logistic regression model were genus level Corynebacterium, genus level Bacteroides, genus level Pseudomonas, Rothia mucilaginosa, and Mycobacterium pneumoniae/genitalium.

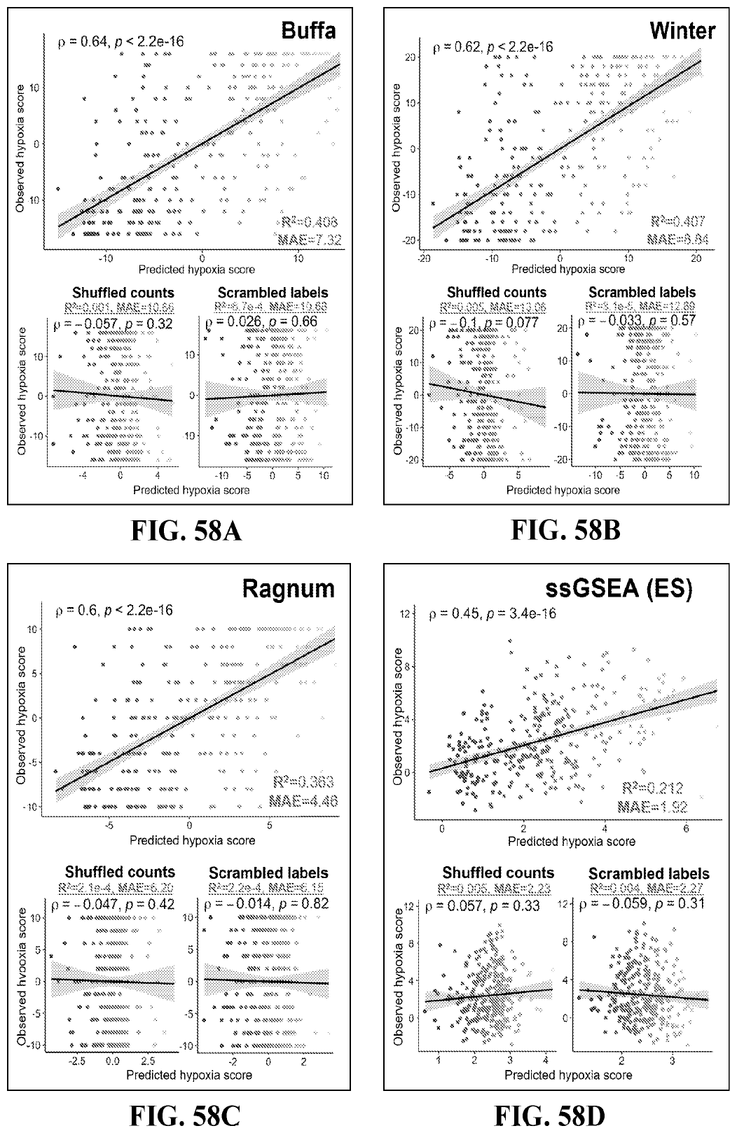

Regression analyses were also conducted to predict the quantitative degree of hypoxia of each AOI based on the AOI's microbial composition with hypoxia scores from Buffa, Winter, Ragnum, and ssGSEA, as shown in FIGS. 58A-58D. From the regression analysis shown in FIGS. 58A-58D, a significant, positive Spearman correlation was found for Buffa, Winter, Ragnum, and ssGSEA trained regression models. Each regression model's corresponding negative control, which either shuffled count data or scrambled hypoxia labels, showed non-significant Spearman correlations and increases in mean average error (MAE), as shown in FIGS. 58A-58D, supporting the aforementioned regression results' veracity. The top five microbial features for the Buffa-labeled, trained regression model were genus level Pseudomonas, Faecalibacterium prausnitzii, genus level Bacteroides, genus level Corynebacterium, Eubacteria (pan-bacteria), and genus level Prevotella. The top five microbial features for the Winter-labeled, trained regression model were genus level Pseudomonas, Faecalibacterium prausnitzii, genus level Bacteroides, Eubacteria (pan-bacteria), genus level Corynebacterium, and genus level Mycobacterium. The top five microbial features for Ragnum-labeled, trained regression model were genus level Pseudomonas, Faecalibacterium prausnitzii, genus level Corynebacterium, genus level genus level Mycobacterium, and genus level Neisseria. The top five microbial features for ssGSEA-labeled, trained regression model were Faecalibacterium prausnitzii, genus level Pseudomonas, genus level Bacteroides, genus level Prevotella, genus level Corynebacterium, and genus level Mycobacterium.

### Decontamination of Microbial Data and Repetition of Hypoxia Classifiers or Regressors

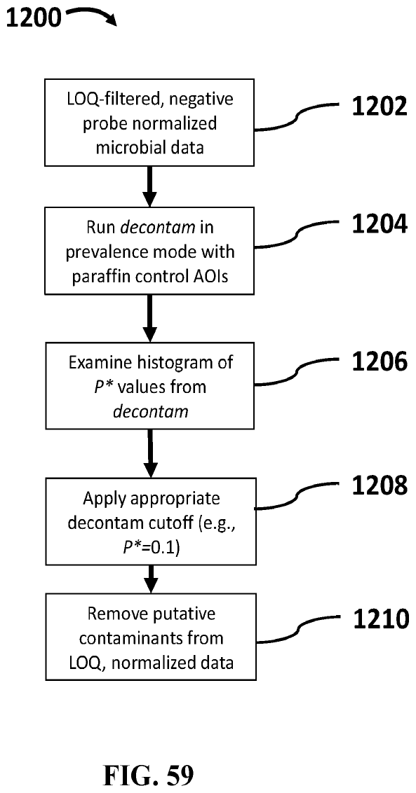

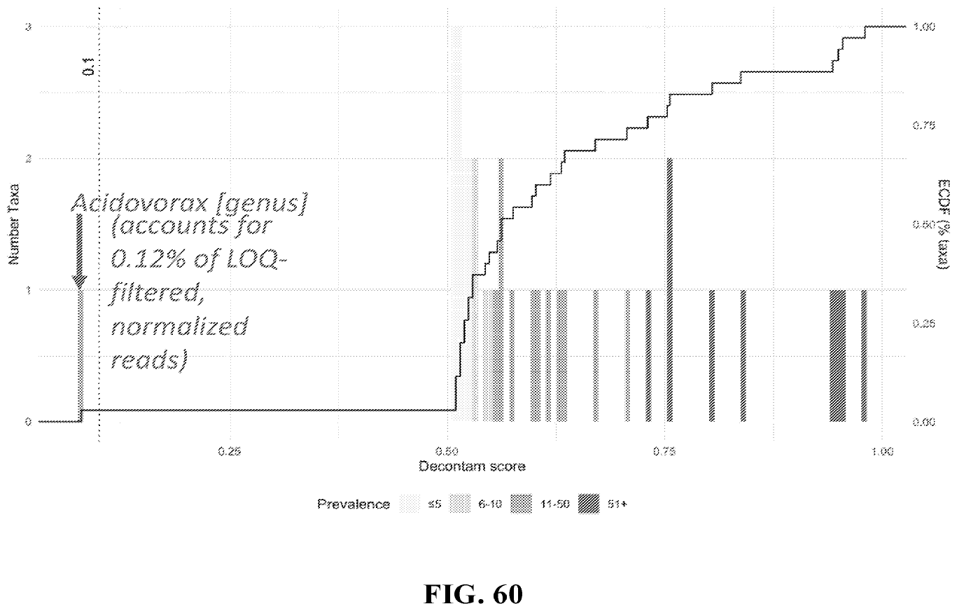

Prevalence-based decontamination of the LOQ-filtered, negative probe normalized microbial data was conducted with decontam to identify putative contaminants (cf. Davis et al. 2018 Microbiome). The method 1200 of decontamination, as shown in FIG. 59, included the following steps: provide LOQ-filtered, negative probe normalized microbial data 1202; run decontam on the LOQ-filtered, negative probe normalized microbial data in prevalence mode with paraffin control AOIs 1204; examine histograms of P* values from decontam 1206; determine the appropriate decontam cutoff (e.g., P*=0.1) and apply it 1208; and remove putative contaminants from LOQ-filtered, negative probe normalized microbial data 1210. By applying the decontamination method 1200 to the microbial data, 1 putative contaminant of genus level Acidovorax was identified in the post-LOQ-filtered data, as shown in FIG. 60 for all P* values between 0.1-0.5. Prevalence mode of decontam compares which microbes have a similar or higher prevalence in the paraffin AOIs compared to the tissue sample AOIs. The genus level Acidovorax accounted for only 0.12% of LOQ-filtered normalized reads. The aforementioned machine learning was repeated with the decontaminated microbial data, with identical resulting AUROC performances between the non-decontaminated and decontaminated data.

### Leave-One-Slide-Out (LOSO) Machine Learning Approach

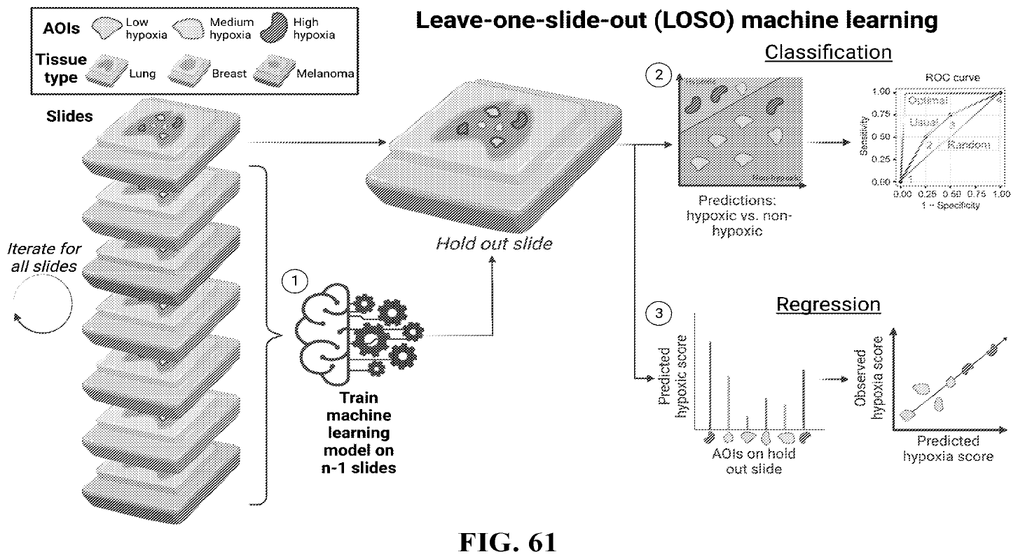

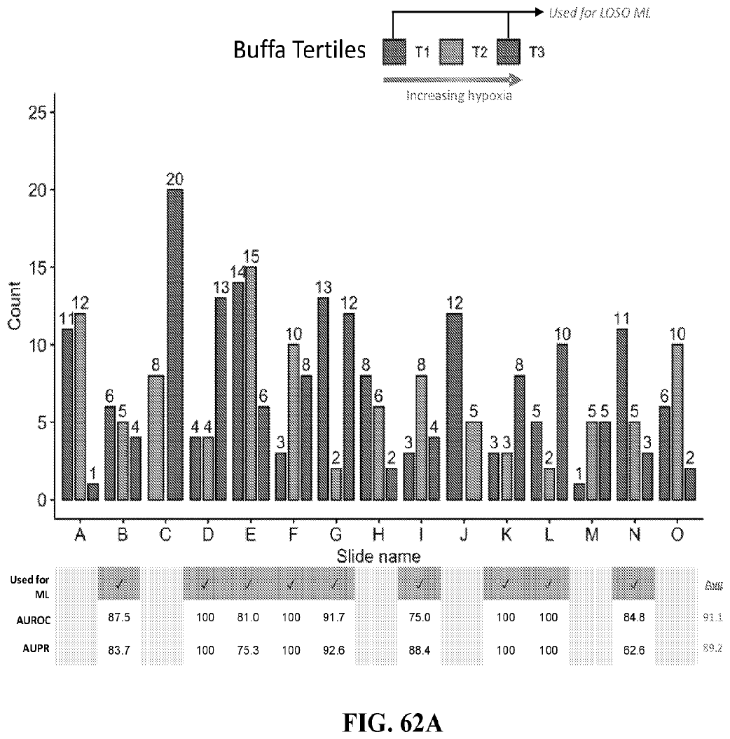

Leave-one-slide-out machine learning was conducted to validate that the machine learning models, described elsewhere herein, are not determining the slide that a given AOI came from and using that slide as a proxy for determining AOI hypoxia. The LOSO machine learning approach, shown in FIG. 61, involved holding out a single slide having multiple AOIs of varying hypoxia and training a classification or regression machine learning model on all other slides across all the tissue types. The classification model was configured to determine a classification boundary between high hypoxia (i.e., the upper third tertile of hypoxia scores) and low hypoxia (i.e., the lower third tertile of hypoxia scores) in a hyper-dimensional space. To ensure stability of the resulting ROC curve areas for the classification model, a requirement of at least 3 AOIs for both high and low hypoxia was implemented as a constraint on the held-out slides. The regression model used all of the AOIs on the held-out slides and measured the prediction quality using rank orders (via Spearman correlations) and mean absolute errors (MAEs). Negative control analyses were also run while, during LOSO training, either scrambling the hypoxia labels/scores or shuffling the microbial count data, followed by evaluating the classification or regression performances.

Leave-One-Slide-Out (LOSO) Classification to Predict Hypoxic Regions with Microbial Compositions

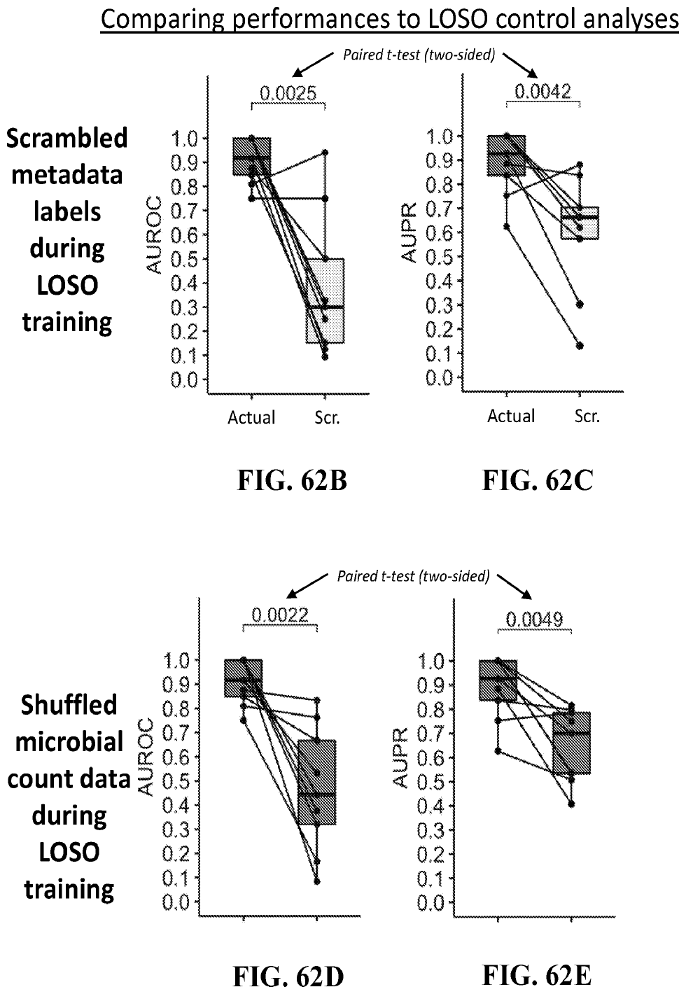

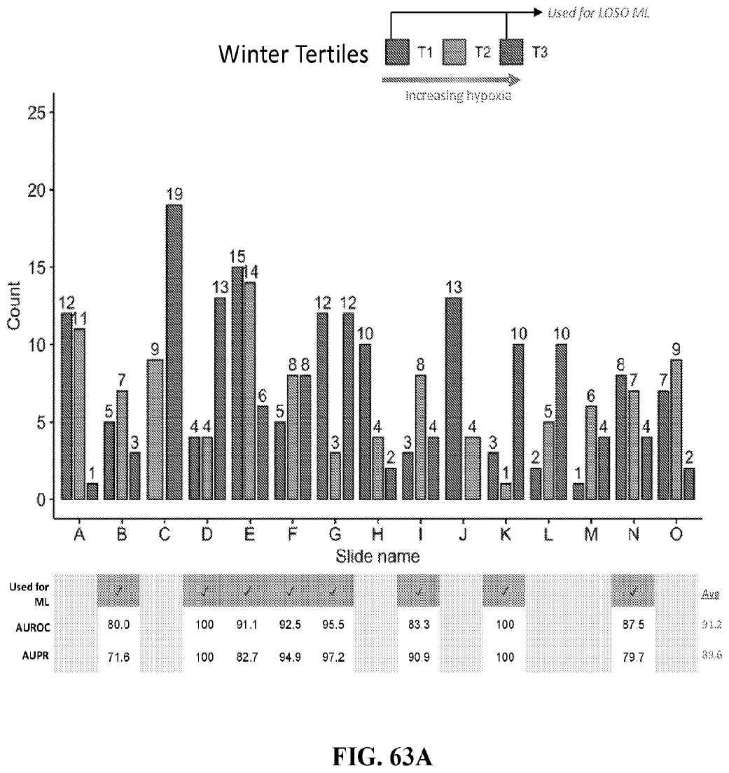

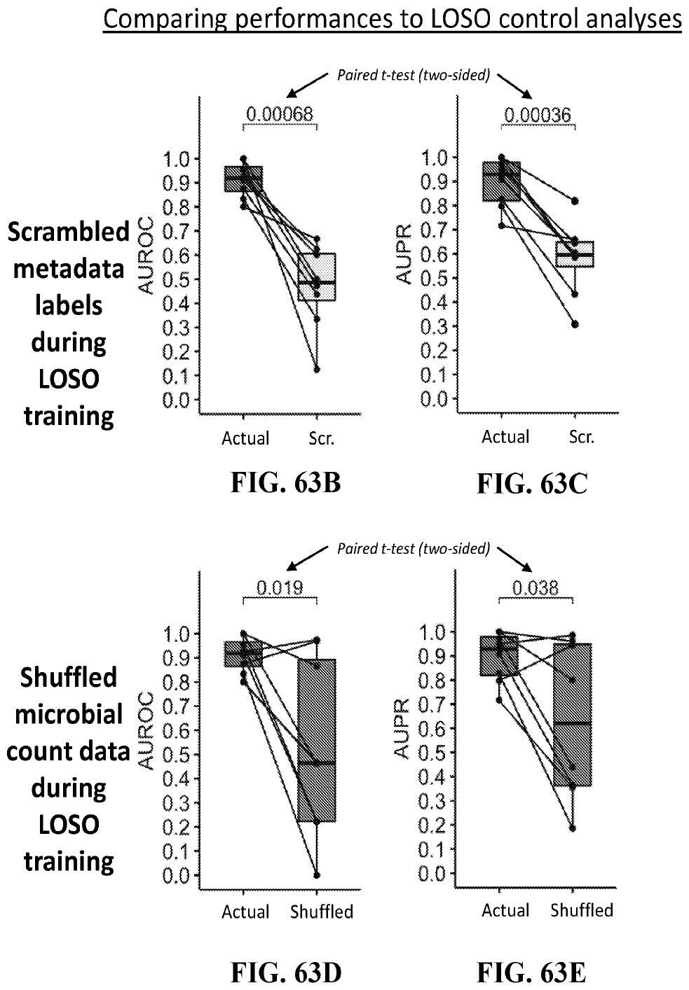

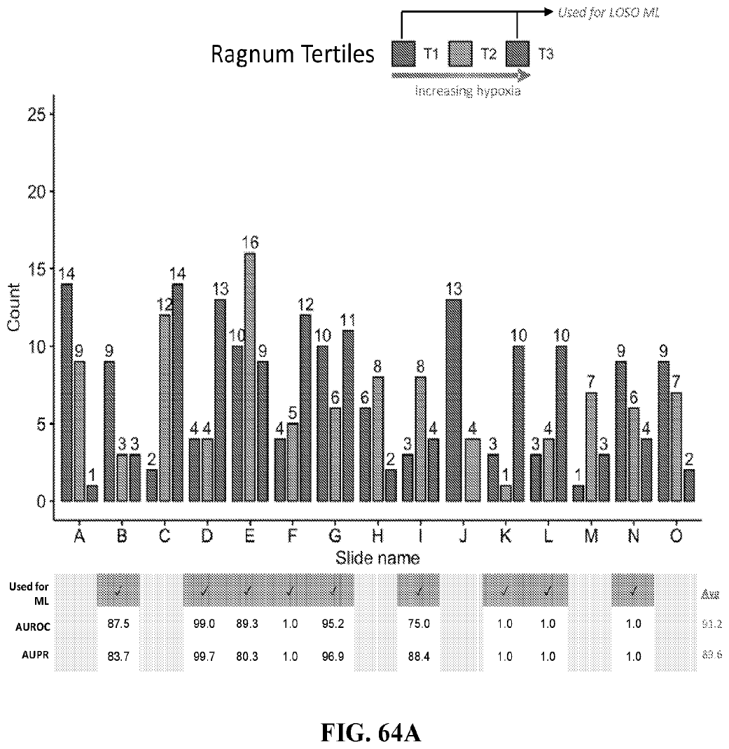

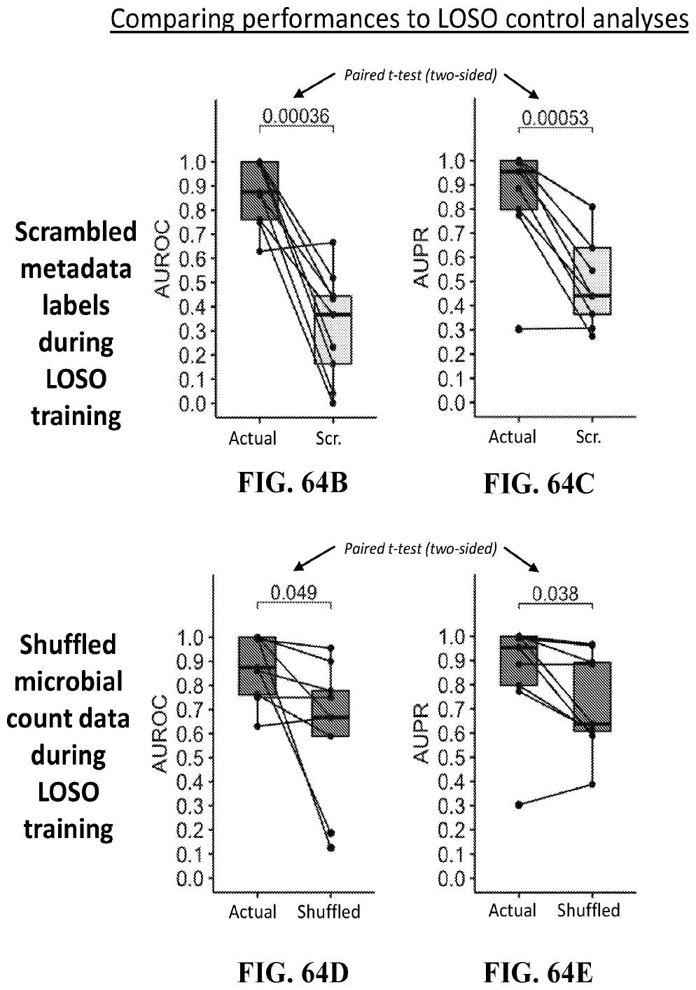

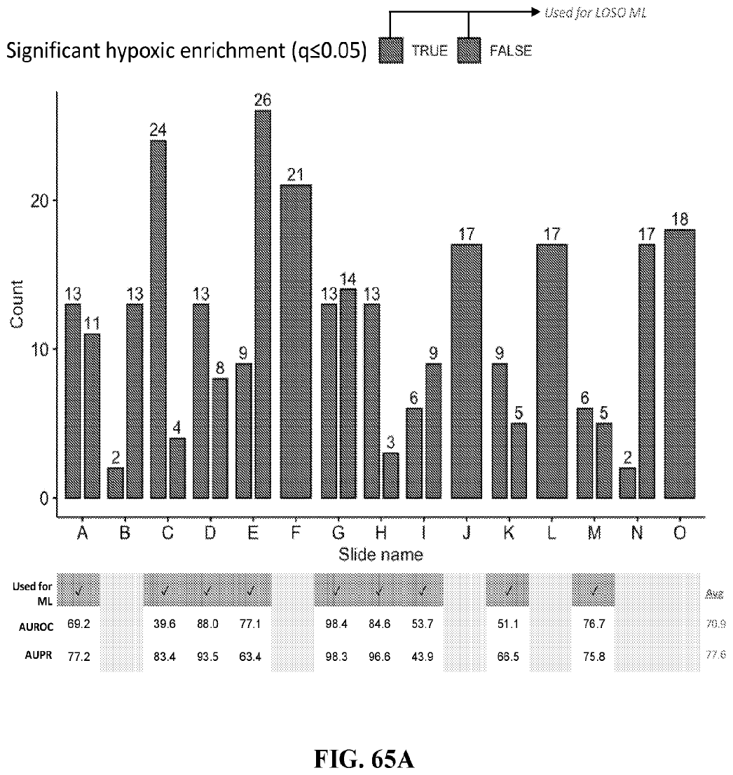

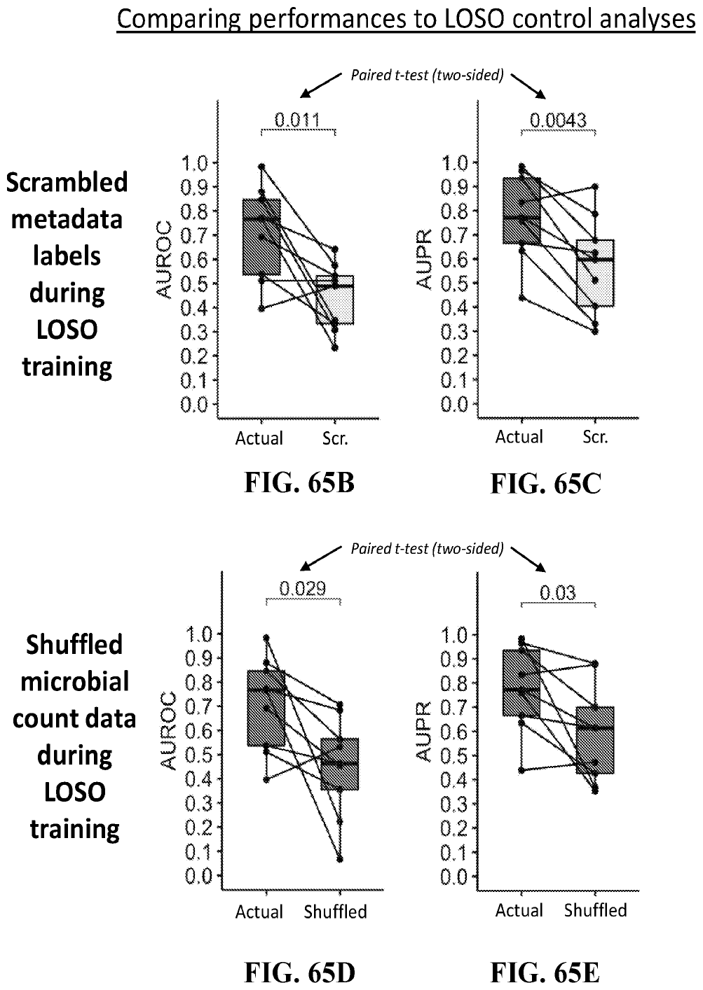

The AUROC and AUPR performance of the trained LOSO classification models in predicting hypoxic categories was determined using Buffa hypoxia scores (FIG. 62A), Winter hypoxia scores (FIG. 63A), Ragnum hypoxia scores (FIG. 64A), and ssGSEA scores (FIG. 65A). The average performance across all slides for the Buffa model was an AUROC of 91.1% and an AUPR of 89.2%. The average performance across all slides for the Winter model was an AUROC of 91.2% and an AUPR of 89.6%. The average performance across all slides for the Ragnum model was an AUROC of 91.2% and an AUPR of 89.6%. The average performance across all slides for the ssGSEA model was an AUROC of 70.9% and an AUPR of 77.6%. Corresponding negative control analyses' AUROCs and AUPRs for Buffa (FIGS. 62B-62E), Winter (FIGS. 63B-63E), Ragnum (FIGS. 64B-64E), and ssGSEA (FIGS. 65B-65E), were compared to the actual performances on held-out slides using a paired two-sided t-test, showing that the control analyses' results were significantly worse, as expected. The data shown from the LOSO classification analyses suggest that the predictions of hypoxia based on the microbial data are not confounded by slide or tissue type.

Leave-One-Slide-Out Regression to Predict Hypoxic Degree with Microbial Compositions

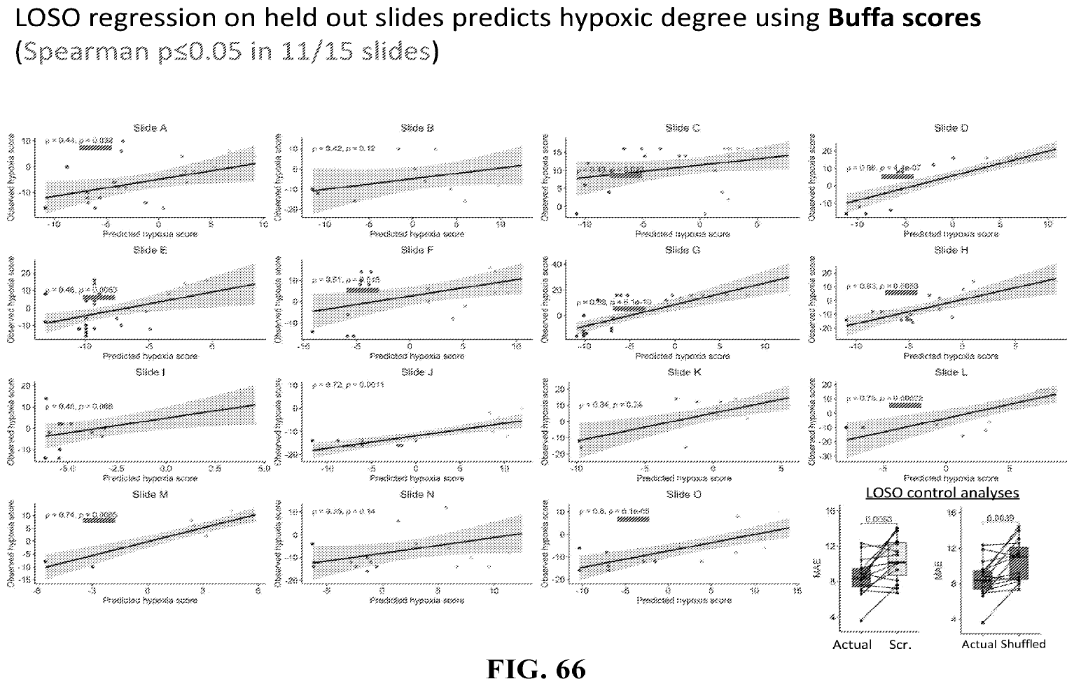

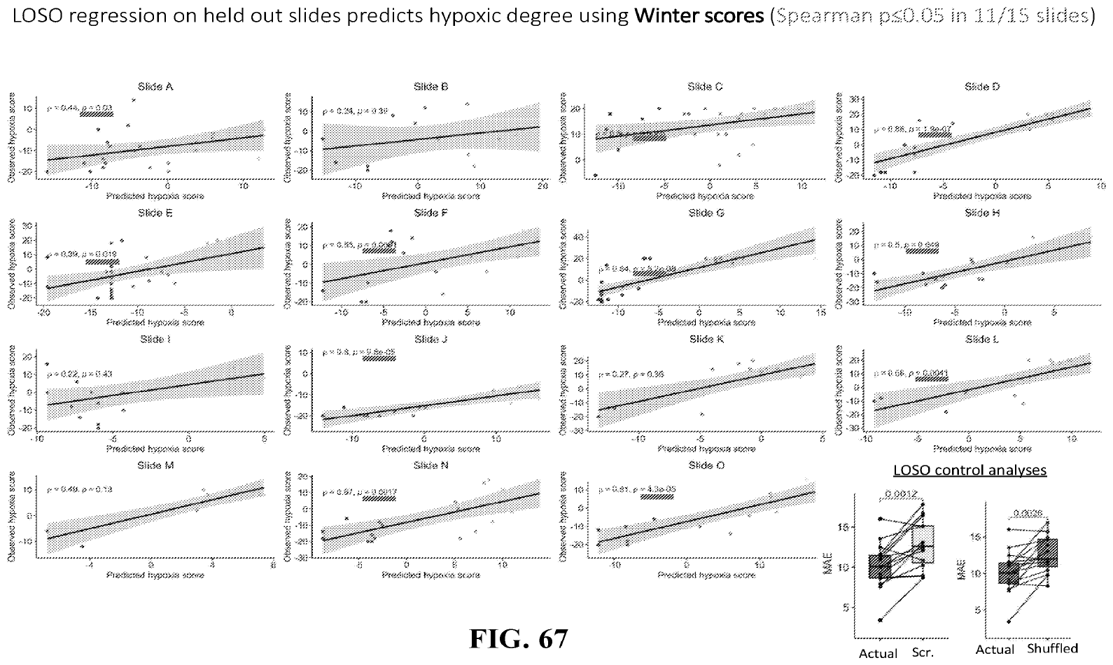

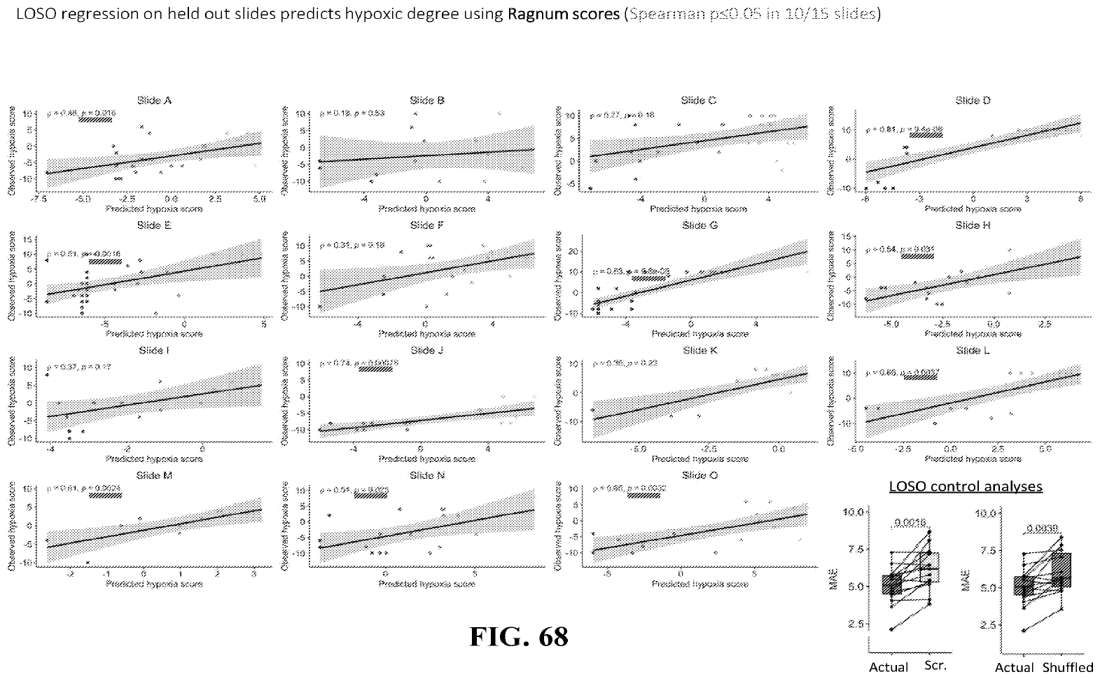

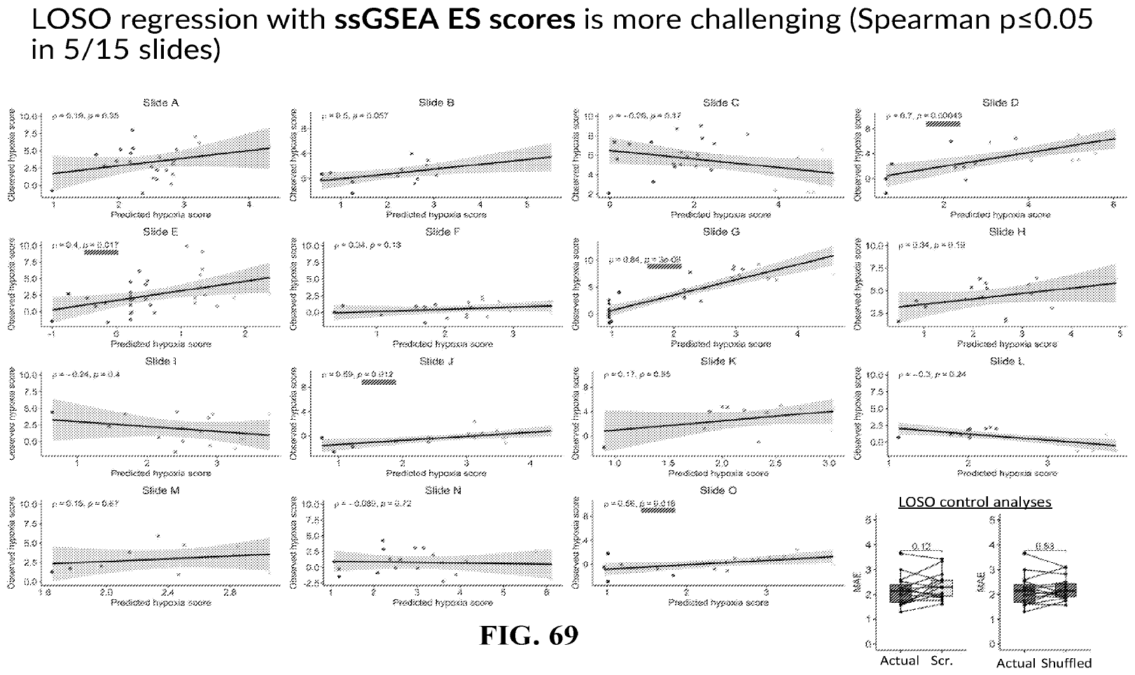

Leave-one-slide-out regression was used to predict the quantitative hypoxic degree using standard gradient boosting machines and Bayesian ridge regression (a regression-specific machine learning model type). LOSO regression with standard gradient boosting machines was used with Buffa (FIG. 66), Winter (FIG. 67), Ragnum (FIG. 68), and ssGSEA (FIG. 69) hypoxia scores. From the regression Spearman correlation p-values shown on each slide's scatter plot within FIGS. 66-69, 11 of 15 slides with Buffa hypoxia scores, 11 of 15 slides with Winter hypoxia scores, 10 of 15 slides with Ragnum scores, and 5 of 15 slides with ssGSEA hypoxia scores were found to be significant. The MAE in the regression analysis for each gradient boosting machine model with Buffa, Winter, Ragnum, and ssGSEA was calculated and compared to actual vs. negative control LOSO analyses (i.e., scrambled metadata or shuffled count data during LOSO training) with a paired two-sided t-test. The MAE increases for the negative control analyses for Buffa, Winter, and Ragnum were found to be significant, whereas it was not found to be significant for ssGSEA.

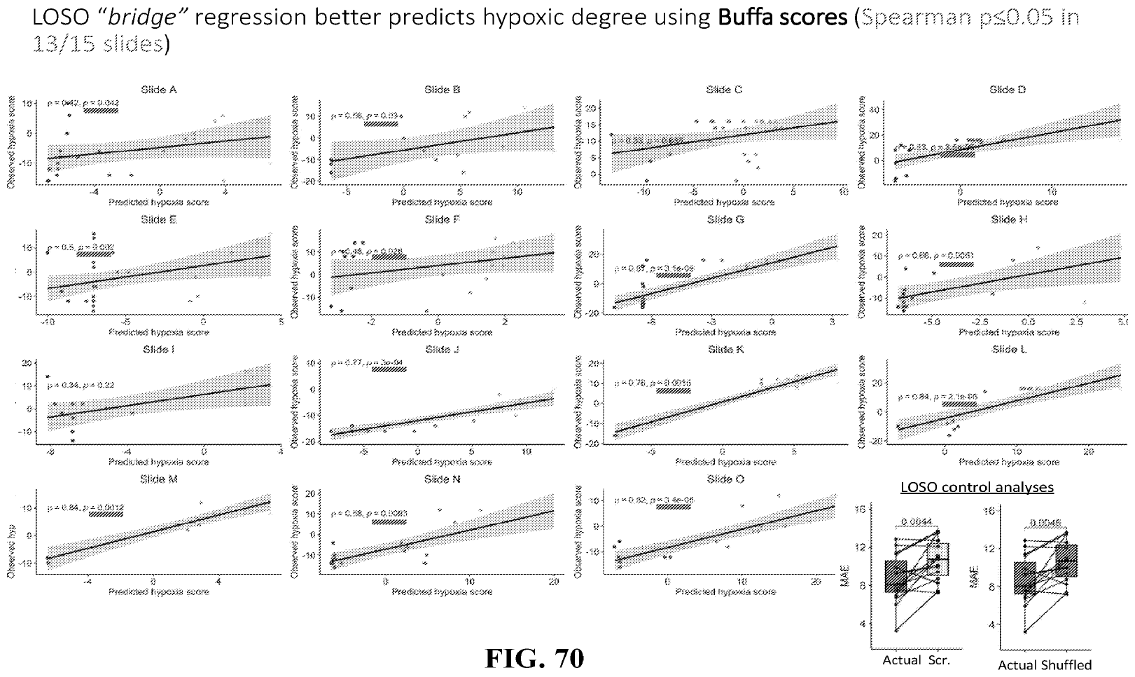

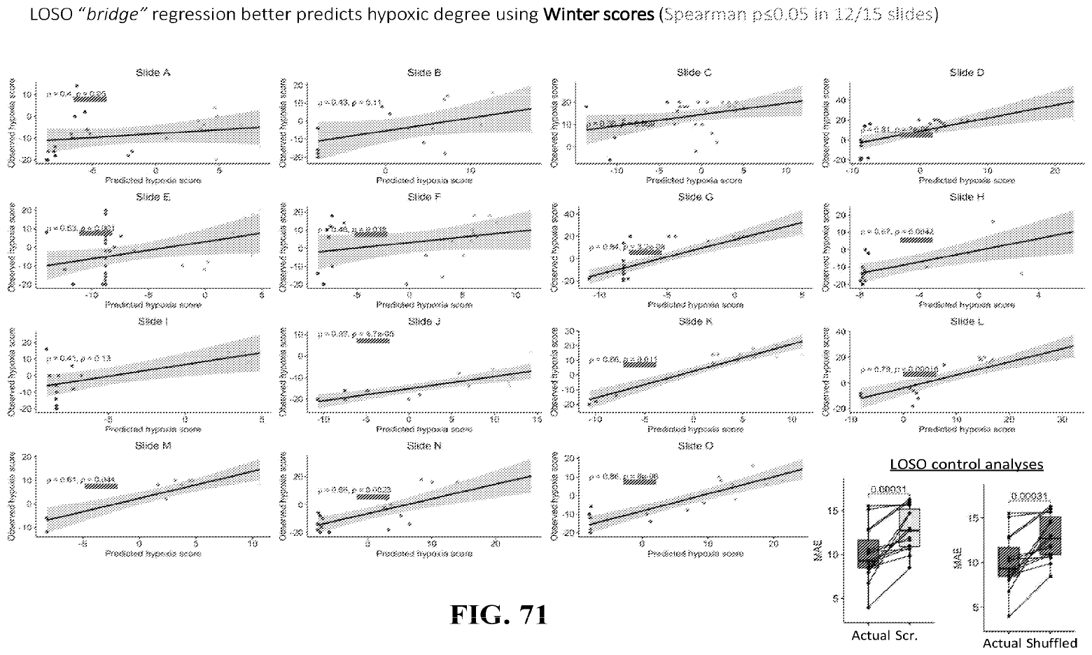

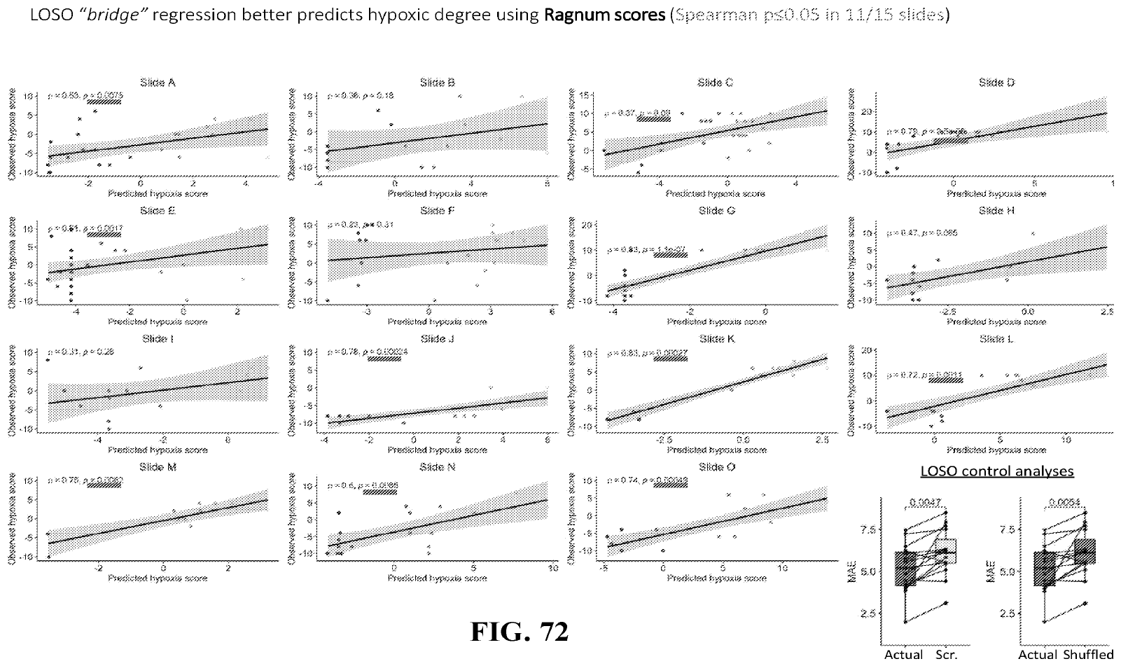

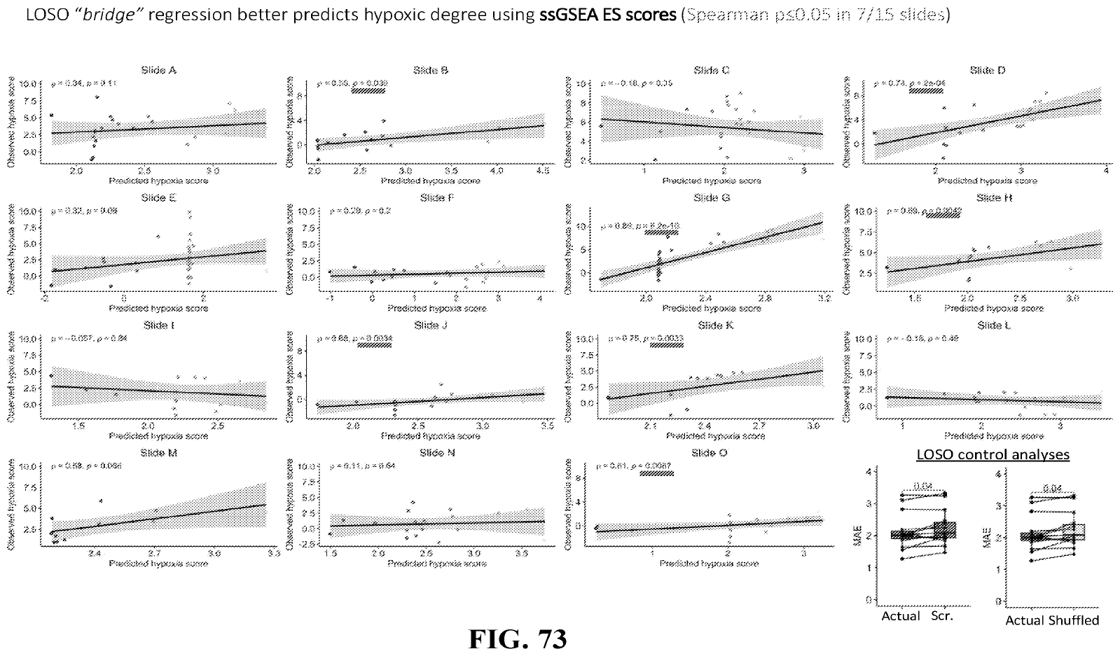

To optimize the regression, Bayesian ridge regression (“bridge regression”) model was used to predict hypoxic degree with microbial compositions. Bayesian ridge regression is a regression-specific model that does not provide classification. LOSO bridge regression was used with Buffa (FIG. 70), Winter (FIG. 71), Ragnum (FIG. 72), and ssGSEA (FIG. 73) hypoxia scores. From the bridge regression Spearman correlation p-values shown on each slide's scatter plot within FIGS. 70-73, 13 of 15 slides with Buffa hypoxia scores, 12 of 15 slides with Winter Hypoxia scores, 11 of 15 slides with Ragnum hypoxia scores, and 7 of 15 slides with ssGSEA hypoxia scores were found to be significant. The MAE in the bridge regression analyses with Buffa, Winter, Ragnum, and ssGSEA scores was calculated and compared to the negative control LOSO analyses (i.e., scrambled metadata or shuffled count data during LOSO training) with a paired two-sided t-test. The MAE increases for the negative control analyses for Buffa, Winter, Ragnum, and ssGSEA were found to be significant.

From the experiments and results described above, the following conclusions and outcomes were reached: (1) approximately 300 AOIs across 15 pre-screened tissue blocks and 3 cancer types were deeply sequenced for human and microbial RNA using spatial transcriptomics; (2) two independent methods were employed to derive AOI hypoxia estimates across four published gene sets with strong correlations; (3) microbial alpha and beta diversities display significant hypoxia-mediated variation commensurate to that observed in similar analyses using human and microbial data from The Cancer Genome Atlas (TCGA); and (4) microbial-derived classifiers can accurately predict the hypoxic category and degree in global and leave-one-slide-out (LOSO) analyses.

1. A method of determining a tumor oxygen characteristic of a subject, comprising:
(a) receiving one or more biological samples of a subject;
(b) sequencing a plurality of nucleic acid molecules of the one or more biological samples, thereby generating a plurality of nucleic acid molecule sequencing reads;
(c) mapping the plurality of nucleic acid molecule sequencing reads to a database of microbial genomes, thereby generating a plurality of microbial nucleic acid molecule reads; and
(d) determining a tumor oxygen characteristic of the subject as an output of a trained predictive model when the plurality of microbial nucleic acid molecule reads is provided as an input to the trained predictive model.

2. The method of claim 1, wherein the plurality of microbial nucleic acid molecules originate from bacterial obligate aerobes, aerobes, facultative aerobes, microaerophiles, aerotolerants, microaerotolerants, facultative anaerobes, anaerobes, obligate anaerobes, or any combination thereof.

3. The method of claim 1, wherein the plurality of nucleic acid molecules comprises microbial DNA, microbial RNA, epigenetic markers on microbial DNA, epigenetic markers on microbial RNA, or any combination thereof.

4. The method of claim 3, wherein the plurality of nucleic acid molecules comprises human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

5. The method of claim 1, further comprising decontaminating the plurality of nucleic acid molecule sequencing reads thereby producing a plurality of decontaminated nucleic acid molecule sequencing reads.

6. The method of claim 5, wherein decontaminating is conducted in silico, using experimental contamination controls, limit of quantification filtering, or any combination thereof.

7. The method of claim 1, wherein the one or more biological samples comprise a tissue biopsy, a liquid biopsy, or any combination thereof.

8. The method of claim 7, wherein the tissue biopsy comprises cancerous tissue, non-cancerous tissue, or any combination thereof.

9. The method of claim 7, wherein the liquid biopsy comprises whole blood, red blood cells, plasma, white blood cells, saliva, urine, tears, breast milk, or any combination thereof.

10. The method of claim 1, wherein the trained predictive model comprises a machine learning model.

11. The method of claim 1, wherein the trained predictive model comprises a regularized machine learning model.

12. The method of claim 1, wherein the trained predictive model comprises one or more machine learning models.

13. The method of claim 1, wherein the trained predictive model comprises a gradient boosting machine, neural network, support vector machine, k-means, classification trees, random forest, regression, or any combination thereof machine learning model.

14. The method of claim 1, wherein the trained predictive model is trained with microbial DNA, microbial RNA, epigenetic marks on microbial DNA, epigenetic marks on microbial RNA, cell-free microbial RNA, cell-free microbial DNA, non-microbial DNA, non-microbial RNA, epigenetic marks on non-microbial DNA, epigenetic marks on non-microbial RNA, non-microbial cell free DNA, non-microbial cell free RNA, or any combination thereof.

15. The method of claim 1, wherein the tumor comprises breast, lung, bone, brain, pancreas, ovarian, colorectal, skin, or any combination thereof tumors.

16. A method of generating a tumor oxygen characteristic predictive model, comprising:
(a) obtaining one or more biological samples of one or more subjects with cancer, and corresponding tumor oxygen characteristics of the one or more subjects;
(b) sequencing a plurality of nucleic acid molecules of the one or more biological samples thereby generating a plurality of nucleic acid molecule sequencing reads;
(c) mapping the plurality of nucleic acid molecule sequencing reads to a database of microbial genomes, thereby generating a plurality of microbial nucleic acid molecule reads; and
(d) generating a tumor oxygen characteristic predictive model by training a predictive model with the plurality of microbial nucleic acid molecule reads and corresponding tumor oxygen characteristics of the one or more subjects.

17. The method of claim 16, wherein the tumor oxygen characteristic is determined by the RNA expression of one or more genes, the presence or absence of epigenetic marks of one or more genes, the staining intensity of one or more proteins, a physical measurement of oxygen concentration, or any combination thereof.

18. The method of claim 16, wherein the plurality of microbial nucleic acid molecule reads originate from bacterial obligate aerobes, aerobes, facultative aerobes, microaerophiles, aerotolerants, microaerotolerants, facultative anaerobes, anaerobes, obligate anaerobes, or any combination thereof.

19. The method of claim 16, wherein the plurality of nucleic acid molecules comprises microbial DNA, microbial RNA, epigenetic markers on microbial DNA, epigenetic markers on microbial RNA, or any combination thereof.

20. The method of claim 19, wherein the plurality of nucleic acid molecules comprises human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

21. The method of claim 16, further comprising decontaminating the plurality of nucleic acid molecule sequencing reads, thereby producing a plurality of decontaminated nucleic acid molecule sequencing reads.

22. The method of claim 21, wherein decontaminating is conducted in silico, using experimental contamination controls, limit of quantification filtering, or any combination thereof.

23. The method of claim 16, wherein the one or more biological samples comprise a tissue biopsy, a liquid biopsy, or any combination thereof.

24. The method of claim 23, wherein the tissue biopsy comprises cancerous tissue, non-cancerous tissue, or any combination thereof.

25. The method of claim 23, wherein the liquid biopsy comprises whole blood, red blood cells, plasma, white blood cells, saliva, urine, tears, breast milk, or any combination thereof.

26. The method of claim 16, wherein, wherein the predictive model comprises a machine learning model.

27. The method of claim 16, wherein the predictive model comprises a regularized machine learning model.

28. The method of claim 16, wherein the predictive model comprises one or more machine learning models.

29. The method of claim 16, wherein the predictive model comprises a gradient boosting machine, neural network, support vector machine, k-means, classification trees, random forest, regression, or any combination thereof machine learning model.

30. The method of claim 16, wherein the cancer and/or tumor of the subject comprises breast, lung, bone, brain, pancreas, ovarian, colorectal, skin, or any combination thereof cancers and/or tumors.

31. A method of generating a tumor oxygen characteristic predictive model, comprising:
(a) obtaining or receiving one or more nucleic acid molecule sequences and corresponding tumor oxygen characteristics of one or more subjects with cancer from a database;
(b) mapping the plurality of nucleic acid molecule sequencing reads to a database of microbial genomes, thereby generating a plurality of microbial nucleic acid molecule reads; and
(c) generating a tumor oxygen characteristic predictive model by training a predictive model with the plurality of microbial nucleic acid molecule reads and corresponding tumor oxygen characteristics of the one or more subjects.

32. The method of claim 31, wherein the tumor oxygen characteristics are determined by the RNA expression of one or more genes, the presence of epigenetic marks of one or more genes, the staining intensity of one or more proteins, a physical measurement of oxygen concentration, or any combination thereof.

33. The method of claim 31, wherein the plurality of microbial nucleic acid molecule reads originate from bacterial obligate aerobes, aerobes, facultative aerobes, microaerophiles, aerotolerants, microaerotolerants, facultative anaerobes, anaerobes, obligate anaerobes, or any combination thereof.

34. The method of claim 31, wherein the one or more nucleic acid molecule sequences comprise microbial DNA, microbial RNA, epigenetic markers on microbial DNA, epigenetic markers on microbial RNA, or any combination thereof.

35. The method of claim 34, wherein the one or more nucleic acid molecule sequences comprise sequences of human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

36. The method of claim 31, further comprising decontaminating the plurality of nucleic acid molecule sequencing reads thereby producing a plurality of decontaminated nucleic acid molecule sequencing reads.

37. The method of claim 36, wherein decontaminating is conducted in silico, using experimental contamination controls, limit of quantification filtering, or any combination thereof.

38. The method of claim 31, wherein the one or more nucleic acid molecule sequences originate from a tissue biopsy, a liquid biopsy, or any combination thereof.

39. The method of claim 38, wherein the tissue biopsy comprises cancerous tissue, non-cancerous tissue, or any combination thereof.

40. The method of claim 38, wherein the liquid biopsy comprises whole blood, red blood cells, plasma, white blood cells, saliva, urine, tears, breast milk, or any combination thereof.

41. The method of claim 31, wherein the predictive model comprises a machine learning model.

42. The method of claim 31, wherein the predictive model comprises a regularized machine learning model.

43. The method of claim 31, wherein the predictive model comprises one or more machine learning models.

44. The method of claim 31, wherein the predictive model comprises a gradient boosting machine, neural network, support vector machine, k-means, classification trees, random forest, regression, or any combination thereof machine learning model.

45. The method of claim 31, wherein the cancer of the subject one or more subjects comprise breast, lung, bone, brain, pancreas, ovarian, colorectal, skin cancers, or any combination thereof cancers.

46. The method of claim 31, wherein the predictive model is configured to provide a prediction of tumor hypoxia, prognosis of survival, prognosis of likelihood of treatment response, or any combination thereof, of the one or more subjects.

47. A method of administering a bacterial theranostic, comprising:
(a) selecting from a database one or more microbes, wherein the one or more microbes comprise a metabolic activity based on oxygen concentrations;
(b) modifying the one or more microbes with one or more reporter genes, thereby producing a modified one or more microbes, wherein the one or more reporter genes when incorporated into the one or more microbes, cause the one or more microbes to secrete one or more metabolites in response to oxygen concentrations; and
(c) administering to a subject a treatment comprising the modified one or more microbes thereby treating the subject's disease.

48. The method of claim 47, wherein the one or more microbes, the one or more metabolites, a product of the one or more reporter genes, or any combination thereof, comprise anticancer properties.

49. The method of claim 47, wherein the one or more metabolites or the product of the one or more reporter genes are detected by non-invasive imaging, invasive imaging, or any combination thereof imaging to diagnose the subject's disease.

50. The method of claim 47 wherein the one or more metabolites or the product of the one or more reporter genes comprise a second set of molecules configured to be detected by blood based, urine detection, or any combination thereof assays.

51. The method of claim 47, wherein the subject's disease comprises cancer.

52. The method of claim 47, wherein the treatment comprises an oral available probiotic, an injection into the subject's tumor, an intramuscular injection, an intravenous injection, or any combination thereof.

53. A method of administering one or more microbes to determine a subject's tumor oxygenation characteristic, comprising:
(a) selecting from a database one or more microbes, wherein the one or more microbes comprise a metabolic activity based on oxygen concentrations;
(b) modifying the one or more microbes with one or more reporter genes, wherein the one or more reporter genes, when incorporated into the one or more microbes, causes the one or more microbes to secrete one or more metabolites or one or more proteins in response to oxygen concentrations; and
(c) administering to a subject with a tumor the one or more microbes, wherein the subject's tumor oxygen characteristic is determined by detecting the one or more secreted metabolites or one or more proteins of the one or more microbes.

54. The method of claim 53, wherein the one or more microbes, the one or more metabolites, the one or more proteins, or any combination thereof, comprise anticancer properties.

55. The method of claim 53, wherein the one or more metabolites or proteins are detected by non-invasive imaging, invasive imaging, or any combination thereof imaging to diagnose the subject's disease, tumor oxygenation characteristic, or any combination thereof.

56. The method of claim 53, wherein the one or more microbes administered to the subject are administered as an oral available probiotic, an injection into the subject's tumor, an intramuscular injection, an intravenous injection, or any combination thereof.

57. The method of claim 53, wherein the one or more metabolites or proteins indicate the prognosis of the subject's disease-free survival, overall survival, likelihood of treatment response, or any combination thereof.

58. A method of providing a treatment to a set of subjects based on tumor oxygenation characteristics, comprising:
(a) receiving a first set of subjects' one or more biological samples and corresponding treatment provided to treat each subject of the first set of subjects' diseases;
(b) sequencing the first set of subjects' plurality of nucleic acid molecules of the one or more biological samples thereby producing a plurality of nucleic acid molecule sequencing reads;
(c) mapping the first set of subjects' plurality of nucleic acid molecule sequencing reads to a database of microbial genomes, thereby generating a plurality of microbial nucleic acid molecule sequencing reads;
(d) training a predictive model with the first set of subjects' plurality of microbial nucleic acid molecule sequencing reads and corresponding treatment provided to each subject of the first set of subjects, thereby generating a trained predictive model;
(e) providing a treatment to treat a second set of subjects' diseases based on the output of the trained predictive model when the trained predictive model is provided, as an input, the second set of subjects' plurality of microbial nucleic acid molecule sequencing reads of the second set of subjects' one or more biological samples.

59. The method of claim 58, wherein the predictive model is trained with the first set of subjects' plurality of microbial nucleic acid molecule sequencing reads and corresponding oxygen concentration values.

60. The method of claim 58, wherein the treatment comprises anti-angiogenic therapies, non-anti-angiogenic therapies, or any combination thereof treatment.

61. The method of claim 58, wherein the first or second set of subjects' diseases comprise cancer.

62. The method of claim 58, wherein the first or second set of subjects' plurality of microbial nucleic acid molecule sequencing reads originate from bacterial obligate aerobes, aerobes, facultative aerobes, microaerophiles, aerotolerants, microaerotolerants, facultative anaerobes, anaerobes, obligate anaerobes, or any combination thereof.

63. The method of claim 58, wherein the first or second set of subjects' plurality of nucleic acid molecules comprise microbial DNA, microbial RNA, epigenetic markers on microbial DNA, epigenetic markers on microbial RNA, or any combination thereof.

64. The method of claim 63, wherein the first or second set of subjects' plurality of nucleic acid molecules comprise human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

65. The method of claim 58, further comprising decontaminating the first or second set of subjects' plurality of microbial nucleic acid molecule sequencing reads thereby producing a plurality of decontaminated microbial nucleic acid molecule sequencing reads.

66. The method of claim 65, wherein decontaminating is conducted in silico, using experimental contamination controls, limit of quantification filtering, or any combination thereof.

67. The method of claim 58, wherein the first or second set of subjects' one or more biological samples comprise a tissue biopsy, a liquid biopsy, or any combination thereof.

68. The method of claim 67, wherein the tissue biopsy comprises cancerous tissue, non-cancerous tissue, or any combination thereof.

69. The method of claim 67, wherein the liquid biopsy comprises whole blood, red blood cells, plasma, white blood cells, saliva, urine, tears, breast milk, or any combination thereof.

70. The method of claim 58, wherein the trained predictive model comprises a machine learning model.

71. The method of claim 58, wherein the trained predictive model comprises a regularized machine learning model.

72. The method of claim 58, wherein the trained predictive model comprises one or more machine learning models, an ensemble of machine learning models, or any combination thereof.

73. The method of claim 58, wherein the trained predictive model comprises a gradient boosting machine, neural network, support vector machine, k-means, classification trees, random forest, regression, or any combination thereof machine learning model.

74. A computer system configured to determine an estimate of tumor oxygenation of a subject, comprising:
(a) one or more processors; and
(b) a non-transient computer readable storage medium including software, wherein the software comprises executable instructions that, as a result of execution, cause the one or more processors of the computer system to:
(i) receive or obtain one or more biological samples of a subject;
(ii) sequence a plurality of nucleic acid molecules of the one or more biological samples thereby generating a plurality of nucleic acid molecule sequencing reads;
(iii) map the plurality of nucleic acid molecule sequencing reads to a database of microbial genomes, thereby generating a plurality of microbial nucleic acid molecule reads; and
(iv) determine an estimate of tumor oxygenation of the subject as an output of a trained predictive model when the plurality of microbial nucleic acid molecule reads are provided as an input to the trained predictive model.

75. The system of claim 74, wherein the plurality of microbial nucleic acid molecules originate from bacterial obligate aerobes, aerobes, facultative aerobes, microaerophiles, aerotolerants, microaerotolerants, facultative anaerobes, anaerobes, obligate anaerobes, or any combination thereof.

76. The system of claim 74, wherein the plurality of nucleic acid molecules comprises microbial DNA, microbial RNA, epigenetic markers on microbial DNA, epigenetic markers on microbial RNA, or any combination thereof.

77. The system of claim 74, wherein the plurality of nucleic acid molecules comprises human RNA, human DNA, cell-free DNA, cell-free RNA, cell-free tumor DNA, cell-free tumor RNA, exosome-derived tumor DNA, exosome-derived tumor RNA, circulating tumor cell derived DNA, circulating tumor cell derived RNA, methylation patterns of cell-free tumor DNA, methylation patterns of cell-free tumor RNA, methylation patterns of circulating tumor cell derived DNA, methylation patterns of circulating tumor cell derived RNA, spatially-resolved DNA, spatially-resolved RNA, or any combination thereof.

78. The system of claim 74, wherein the instructions further comprise decontaminate the plurality of nucleic acid molecule sequencing reads thereby producing a plurality of decontaminated nucleic acid molecule sequencing reads.

79. The system of claim 78, wherein the decontamination is conducted in silico, using experimental contamination controls, limit of quantification filtering, or any combination thereof.

80. The system of claim 74, wherein the one or more biological samples comprise a tissue biopsy, a liquid biopsy, or any combination thereof.

81. The system of claim 80, wherein the tissue biopsy comprises cancerous tissue, non-cancerous tissue, or any combination thereof.

82. The system of claim 80, wherein the liquid biopsy comprises whole blood, red blood cells, plasma, white blood cells, saliva, urine, tears, breast milk, or any combination thereof.

83. The system of claim 74, wherein the trained predictive model comprises one or more machine learning models, an ensemble of machine learning models, or any combination thereof.

84. The system of claim 74, wherein the trained predictive model comprises a regularized machine learning model.

85. The system of claim 74, wherein the trained predictive model comprises a gradient boosting machine, neural network, support vector machine, k-means, classification trees, random forest, regression, or any combination thereof machine learning model.

86. The system of claim 74, wherein the trained predictive model is trained with microbial DNA, microbial RNA, epigenetic marks on microbial DNA, epigenetic marks on microbial RNA, cell-free microbial RNA, cell-free microbial DNA, non-microbial DNA, non-microbial RNA, epigenetic marks on non-microbial DNA, epigenetic marks on non-microbial RNA, non-microbial cell free DNA, non-microbial cell free RNA, or any combination thereof.

87. The system of claim 74, wherein the tumor comprises breast, lung, bone, brain, pancreas, ovarian, colorectal, skin, or any combination thereof cancers.
